SEQUENCE LISTING (20 sequences)

SEQ ID NO: 1 - protein, 262 aa, synthetic construct
  REGION 1..262: note = Synthetic
AMYPDLKGKVVAITGAASGLGKAMAIRFGKEQAKVVINYYSNKQDPNEVKEEVIKAGGEAVVVQGDVTKEEDVKNIVQTAIKEFGTLDIMINNAGLFNPVPSHEMPLKDWDKVIGTNLTGAFLGSREAIKYFVENDIKGNVINMSSVHEVIPWPLASHYTASKGGMKLMTKTLALEYAPKGIRVNNIGPGAINTTINAEKFADPKQKADVESMIPMGYIGEPEEIAAVAAWLASKEASYVTGITLFADGGMTLYPSFQAGRG

SEQ ID NO: 2 - protein, 261 aa, synthetic construct
  REGION 1..261: note = Synthetic
MYPDLKGKVVAITGAASGLGKAMAIRFGKEQAKVVINYYSNKQDPNEVKEEVIKAGGEAVVVQGDVTKEEDVKNIVQTAIKEFGTLDIMINNAGLCNLVPSHEMPLKDWDKVIGTNLTGAFLGSREAIKYFVENDIKGNVINMSSVHEVIPWPLASHYTASKGGMKLMTKTLALEYAPKGIRVNNIGPGAINTTINAEKFADPKQKADVESMIPMGYIGEPEEIAAVAAWLASKEASYVTGITLFADGGMTLYPSFQAGRG

SEQ ID NO: 3 - protein, 261 aa, synthetic construct
  REGION 1..261: note = Synthetic
MYPDLKGKVVAITGAASGLGKAMAIRFGKEQAKVVINYYSNKQDPNEVKEEVIKAGGEAVVVQGDVTKEEDVKNIVQTAIKEFGTLDIMINNAGLCNPVPSHEMPLKDWDKVIGTNLTGAFLGSREAIKYFVENDIKGNVINMSSVHEVIPWPLASHYTASKGGMKLMTKTLALEYAPKGIRVNNIGPGAINTTINAEKFADPKQKADVESMIPMGYITEPEEIAAVAAWLASKEASYVTGITLFADGGMTLYPSFQAGRG

SEQ ID NO: 4 - protein, 261 aa, synthetic construct
  REGION 1..261: note = Synthetic
MYPDLKGKVVAITGAASGLGKAMAIRFGKEQAKVVINYYSNKQDPNEVKEEVIKAGGEAVVVQGDVTKEEDVKNIVQTAIKEFGTLDIMINNAGLCNPVPSHEMPLKDWDKVIGTNLTGAFLGSREAIKYFVENDIKGNVINMSSVHEVIPWPLASHYTASKGGMKLMTKTLALEYAPKGIRVNNIGPGAINTTRNAEKFADPKQKADVESMIPMGYIGEPEEIAAVAAWLASKEASYVTGITLFADGGMTLYPSFQAGRG

SEQ ID NO: 5 - protein, 261 aa, synthetic construct
  REGION 1..261: note = Synthetic
MYPDLKGKVVAITGAASGLGKAMAIRFGKEQAKVVINYYSNKQDNNEVKEEVIKAGGEAVVVQGDVTKEEDVKNIVQTAIKEFGTLDIMINNAGLFNPVPSHEMPLKDWDKVIGTNLTGAFLGSREAIKYFVENDIKGNVINMSSVHEAIPWPLASHYTASKGGMKLMTKTLALEYAPKGIRVNNIGPGAINTTINAEKFADPKQKADVESMIPMGYIGEPEEIAAVAAWLASKEASYVTGITLFADGGMTLYPSFQAGRG

SEQ ID NO: 6 - protein, 261 aa, synthetic construct
  REGION 1..261: note = Synthetic
MYPDLKGKVVAITGAASGLGKGMAIRFGKEQAKVVINYYSNKQDPNEVKEEVIKAGGEAVVVQGDVTKEEDVKNIVQTAIKEFGTLDIMINNAGLFNPVPSHEMPLKDWDKVIGTNLTGAFLGSREAIKYFVENDIKGNVINMSSVHEVIPWPGASHYTASKGGMKLMTKTLALEYAPKGIRVNNIGPGAINTTINAEKFADPKQKADVESMIPMGYIGEPEEIAAVAAWLASKEASYVTGITLFADGGMTLYPSFQAGRG

SEQ ID NO: 7 - protein, 261 aa, synthetic construct
  REGION 1..261: note = Synthetic
MYPDLKGKVVAITGAASGLGKAMAIRFGKEQAKVVINYYSNKQDPNEVKEEVIKAGGEAVVVQGDVTKEEDVKNIVQTAIKEFGTLDIMINNAGLFNPVPSHEMPLKDWDKVIGTNLTGAFLGSREAIKYFVENDIKGNVVNMSSVHEVIPWPLASHYTASKGGMKLMTKTLALEYAPKGIRVNNIGPGAINTTINAEKFADPKQKADVESMIPMGYIGEPEEIAAVAAWLASKEASYVTGITLFADGGMTLYPSFQAGRG

SEQ ID NO: 8 - protein, 261 aa, synthetic construct
  REGION 1..261: note = Synthetic
MYPDLKGKVVAITGAASGLGKAMAIRFGKEQAKVVINYYSNKQDPNEVKEEVIKAGGEAVVVQGDVTKEEDVKNIVQTAIKEFGTLDIMINNAGLFNPVPSHEMPLKDWDKVIGTNLTGAFLGSREAIKYFVENDIKGNVVNMSSVHEVIPWPGASHYTASKGGMKLMTKTLALEYAPKGIRVNNIGPGAINTTINAEKFADPKQKADVESMIPMGYIGEPEEIAAVAAWLASKEASYVTGITLFADGGMTLYPSFQAGRG

SEQ ID NO: 9 - protein, 261 aa, synthetic construct
  REGION 1..261: note = Synthetic
MYPDLKGKVVALTGAASGLGKAMAIRFGKEQAKVVINYYSNKQDPNEVKEEVIKAGGEAVVVQGDVTKEEDVKNIVQTAIKEFGTLDIMINNAGLFNPVPSHEMPLKDWDKVIGTNLTGAFLGSREAIKYFVENDIKGNVINMSSVHEVIPWPLASHYTASKGGMKLMTKTLALEYAPKGIRVNNIGPGAINTTRNAEKFADPKQKADVESMIPMGYIGEPEEIAAVAAWLASKEASYVTGITLFADGGMLLYPSFQAGRG

SEQ ID NO: 10 - protein, 261 aa, synthetic construct; identical to SEQ ID NO: 2
  REGION 1..261: note = Synthetic

SEQ ID NO: 11 - protein, 261 aa, synthetic construct
  REGION 1..261: note = Synthetic
MYPDLKGKVVAITGAASGLGKAMAIRFGKEQAKVVINYYSNKQDPNEVKEEVIKAGGEAVVVQGDVTKEEDVKNIVQTAIKEFGTLDIMINNAGLCNPVPSHEMPLKDWDKVIGTNLTGAFLGSREAIKYFVENDIKGNVINMSSVHEVIPWPLASHYTASKGGMKLMTKTLALEYAPKGIRVNNIGPGAINTTINAEKFADPKQKADVESMIPMGYIGEPEEIAAVAAWLASKEASYVTGITLFADGGMTLYPSFQAGRG

SEQ ID NO: 12 - protein, 261 aa, synthetic construct
  REGION 1..261: note = Synthetic
MYPDLKGKVVAITGAASGLGKAMAIRFGKEQAKVVINYYSNKQDPNEVKEEVIKAGGEAVVVQGDVTKEEDVKNIVQTAIKEFGTLDIMINNAGLFNPVPSHEMPLKDWDKVIGTNLTGAFLGSREAIKYFVENDIKGNVINMSSVHEVIPWPLASHYTASKGGMKLMTKTLGLEYAPKGIRVNNIGPGAINTTINAEKFADPKQKADVESMIPMGYIGEPEEIAAVAAWLASKEASYVTGITLFADGGMTLYPSFQAGRG

SEQ ID NO: 13 - protein, 261 aa, synthetic construct
  REGION 1..261: note = Synthetic
MYPDLKGKVVAITGAASGLGKAMAIRFGKEQAKVVINYYSNKQDPNEVKEEVIKEGGEAVVVQGDVTKEEDVKNIVQTAIKEFGTLDIMINNAGLFNPVPSHEMPLKDWDKVIGTNLTGAFLGSREAIKYFVENDIKGNVINMSSVHEVIPWPLASHYTASKGGMKLMTKTLALEYAPKGIRVNNIGPGAINTTINAEKFADPKQKADVESMIPMGYIGEPEEIAAVAAWLASKEASYVTGITLFADGGMTLYPSFQAGRG

SEQ ID NO: 14 - protein, 261 aa, synthetic construct
  REGION 1..261: note = Synthetic
MYPDLKGKVVAITGAASGLGKAMAIRFGKEQAKVVINYYSNKQDPNEVKEEVIKAGGEAVVVQGDVTKEEDVKNIVQTAIKEFGTLDIMINNAGLFNPVPSHEMPLKDWDKVIGTNLTGAFLGSREAIKYFVENDIYGNVINMSSVHEVIPWPLASHYTASKGGMKLMTKTLALEYAPKGIRVNNIGPGAINTTINAEKFADPKQKADVESMIPMGYIGEPEEIAAVAAWLASKEASYVTGITLFADGGMTLYPSFQAGRG

SEQ ID NO: 15 - protein, 261 aa, synthetic construct
  REGION 1..261: note = Synthetic
MYPDLKGKVVAITGAASGLGKAMAIRFGKEQAKVVINYYSNKQDPNEVKEEVIKAGGEAVVVQGDVTKEEDVKNIVQTAIKEFGTLDIMINNAGLFNPVPSHEMPLKDWDKVIGTNLTGAFLGSREAIKYFVENDIKGNVINMSSVHEVIPWPLASHYTASKGGMKLMTKTLALEYAPKGIRVNNIGPGAINTTINAEKFADPKQKADVESMIPMGYIGEPEEIAAVAAWLASKEASYVTGITLFADGGMTVYPSFQAGRG

SEQ ID NO: 16 - protein, 261 aa, synthetic construct
  REGION 1..261: note = Synthetic
MYPDLKGKVVAITGAASGLGKAMAIRFGKEQAKVVINYYSNKQDPNEAKEEVIKAGGEAVVVQGDVTKEEDVKNIVQTAIKEFGTLDIMINNAGLFNPVPSHEMPLKDWDKVIGTNLTGAFLGSREAIKYFVENDIKGNVINMSSVHEVIPWPLASHYTASKGGMKLMTKTLALEYAPKGIRVNNIGPGAINTTINAEKFADPKQKADVESMIPMGYIGEPEEIAAVAAWLASKEASYVTGITLFADGGMTLYPSFQAGRG

SEQ ID NO: 17 - protein, 261 aa, synthetic construct
  REGION 1..261: note = Synthetic
MYPDLKGKVVAITGAASGLGKAMAIRFGKEQAKVVINYYSNKQDPNEVKEEVIKAGGEAVVVQGDVTKEEDVKNIVQTAIKEFGTLDIMINNAGLFNPVPSHEMPLKDWDKVIGTNLTGAFLGSREAIKYFVENDIKGNVINMSSVHEVIPWPLASHYTASKGGMKLMTKTLALEYAPKGIRVSNIGPGAINTTINAEKFADPKQKADVESMIPMGYIGEPEEIAAVAAWLASKEASYVTGITLFADGGMTLYPSFQAGRG

SEQ ID NO: 18 - protein, 261 aa, synthetic construct
  REGION 1..261: note = Synthetic
MYPDLKGKVVAITGAASGLGKAMAIRFGKEQAKVVINYYSNKQDPNEVKEEVIKAGGEAVVVQGDVTKEEDVKNIVQTAIKEFGTLDIMINNAGLFNPVPSHEMPLKDWDKVIGTNLTGAFLGSREAIKYFVENDIKGNVINMSSVHEVIPWPLASHYTASKGGMSLMTKTLALEYAPKGIRVNNIGPGAINTTINAEKFADPKQKADVESMIPMGYIGEPEEIAAVAAWLASKEASYVTGITLFADGGMTLYPSFQAGRG

SEQ ID NO: 19 - protein, 261 aa, synthetic construct
  REGION 1..261: note = Synthetic
MYPDLKGKVVAITGAASGLGKAMAIRFGKEQAKVVINYYSNKQDPNEVKEEVIKAGGEAVVVQGDVTKEEDVKNIVQTAIKEFGTLDIMINNAGLFNPVPSHEMPLKDWDKVIGTNLTGAFLGSREAIKYFVENDIKGNVINMSSVHEVIPWPLASHYTASKGGMKLMTKTLALEYAPKGIRVNNIGPGAINTTINAEKFADPKQKADVESMIPMGYIGEPEEIAAVAAWLASKEASYVQGITLFADGGMTLYPSFQAGRG

SEQ ID NO: 20 - protein, 261 aa, synthetic construct
  REGION 1..261: note = Synthetic
MYPDLKGKVVAITGAASGLGKAMAIRFGKEQAKVVINYYSNKQDPNEVKEEVIKAGGEAVVVQGDVTKEEDVKNIVQTAIKEFGTLDIMINNAGLENPVPSHEMPLKDWDKVIGTNLTGAFLGSREAIKYFVENDIKGNVINMSSVHEVIPWPLFVHYAASKGGMKLMTETLALEYAPKGIRVNNIGPGAINTTINAGKFADPKQKADVESMIPMGYIGEPEEIAAVAAWLASKEASYVTGITLFADGGMTQYPSFQAGRG

(19) United States
(12) Patent Application Publication
(10) Pub. No.: US 2026/0043064 A1
(43) Pub. Date: Feb. 12, 2026

(54) DETECTION OF ANALYTES BY PROTEIN SWITCHES

(71) Applicant: Abbott Diabetes Care Inc., Alameda, CA (US)

(72) Inventors: Anthony Muerhoff, Abbott Park, IL (US); Thomas Leary, Abbott Park, IL (US); Christopher Marohnic, Abbott Park, IL (US); Barry Kreutz, Abbott Park, IL (US); Josie Corby, Abbott Park, IL (US); Benjamin Feldman, Abbott Park, IL (US); Tianmei Ouyang, Abbott Park, IL (US); Ahmed Wali, Abbott Park, IL (US); Oscar Alvizo, Fremont, CA (US)

(21) Appl. No.: 19/302,455

(22) Filed: Aug. 18, 2025

Related U.S. Application Data
(62) Division of application No. 17/763,647.
(60) Provisional application No. 62/909,411, filed on Oct. 2, 2019.

(51) Int. Cl. C12Q 1/32; C12Q 1/00; G01N 33/94

(52) U.S. Cl. CPC C12Q 1/32; C12Q 1/005; G01N 33/94

(57) ABSTRACT

Compositions of matter, methods, devices, systems and apparatus for detecting analytes are disclosed including, for example, protein switches and their use in an in vivo sensor. The protein switch can be used to determine a level of an analyte that is diagnostic for health and/or well-being of a subject.

### CROSS-REFERENCE TO RELATED APPLICATIONS

This application is a divisional application of U.S. application Ser. No. 17/763,647, filed on Mar. 24, 2022, which is a 371 U.S. National Phase application of International Application No. PCT/US2020/053819, filed on Oct. 1, 2020, which claims priority to U.S. Application No. 62/909,411, filed on Oct. 2, 2019, the contents of which are herein incorporated by reference.

### INCORPORATION-BY-REFERENCE OF MATERIAL SUBMITTED ELECTRONICALLY

The instant application contains a Sequence Listing which has been submitted electronically and is hereby incorporated by reference in its entirety. Said XML copy, created on Aug. 18, 2025, is named ABBTD-37810-404.xml and is 32,768 bytes in size.

### FIELD

The present disclosure relates to compositions of matter, methods, devices, systems and apparatus, such as, for example, protein switches and their use in an in vivo sensor, for detecting and/or monitoring analytes. The protein switch can be used to determine a level of one or more analytes that are diagnostic and/or prognostic for health and/or well-being of a subject.

### BACKGROUND

Detecting or monitoring of target analytes is beneficial to the health and/or well-being of certain individuals. Many disease conditions involve analytes that can be measured to diagnose and/or monitor the disease state of an individual. For example, vital organs such as the brain, heart, kidney and liver, as well as neurological and endocrine systems, can be monitored by such analytes.

Key requirements for analyte detection and monitoring are the specificity and sensitivity of an assay. These requirements are particularly important when the target analyte is present in a small or limiting amount or concentration in a fluid or biological sample. Generally, the specificity and/or sensitivity of an assay are provided by the capture and/or detection antibodies used to detect one or more analytes of interest. Diagnostic assays utilizing this approach are well-known and widely used in enzyme-linked immunosorbent sandwich assays (ELISA) or immunoassays generally. It can be challenging (and expensive) to produce antibodies that provide sufficient specificity and/or sensitivity for some analytes.

Current assays to detect target analytes for prognostic, diagnostic and/or monitoring purposes suffer from several limitations which restrict their widespread application in clinical, non-clinical (e.g., wearable), and point-of-care settings. Additionally, many of these assays require a significant level of technical expertise and a panel of expensive and specific reagents (such as antibodies) along with elaborate biomedical infrastructures which typically are housed in specialized laboratory environments.

Consequently, there is a need for compositions, devices, systems, and methods that can detect target analytes for prognostic, diagnostic and/or monitoring purposes in a subject that are relatively inexpensive to produce, capable of measuring small or limiting amounts or concentrations of analyte in a fluid or biological sample and amenable to widespread application in clinical, non-clinical and point-of-care settings.

### SUMMARY

In one embodiment, the present disclosure relates to a protein switch. The protein switch of the present disclosure comprises at least one non-naturally occurring polypeptide having (a) at least one analyte binding domain capable of binding with at least one analyte; and (b) at least one oxidase or dehydrogenase domain having oxidase or dehydrogenase activity and capable of binding or reacting with at least one reactant, wherein (i) the analyte that binds to the analyte binding domain is different than the reactant that binds or reacts with the oxidase or dehydrogenase domain; and (ii) when the analyte binds to the analyte binding domain the oxidase or dehydrogenase activity changes. In a further aspect, the oxidase is glucose oxidase or lactate oxidase. In another aspect, the dehydrogenase is glucose dehydrogenase or lactate dehydrogenase.

In a further aspect, in the above protein switch, when the analyte binding domain binds to the analyte, the activity of the oxidase or dehydrogenase is decreased. In yet a further aspect, in the above protein switch, when the analyte binding domain binds to the analyte, the activity of the oxidase or dehydrogenase is increased. In still yet a further aspect, when the analyte binding domain binds to the analyte, the activity of the oxidase or dehydrogenase is increased or decreased as a result of competitive inhibition, uncompetitive inhibition, or non-competitive inhibition. In still yet another aspect, when the analyte binding domain binds to the analyte, the activity of the oxidase or dehydrogenase is decreased as a result of competitive inhibition.

In yet another aspect, in any of the above described protein switches, the analyte is warfarin, cortisol, methotrexate, or triiodothyronine.

In still yet a further aspect, in any of the above described protein switches, the reactant is glucose or lactate.

In another embodiment, the present disclosure relates to a protein switch comprising at least 7 mutations at amino acid positions 96, 155, 156, 159, 170, 198 and 252 of SEQ ID NO:20.

In some aspects, the protein switch comprises at least the following mutations: (a) a cysteine, phenylalanine, methionine, tryptophan or tyrosine at amino acid position 96 of SEQ ID NO:20; (b) an alanine, glycine, isoleucine, leucine or valine at amino acid position 155 of SEQ ID NO:20; (c) a threonine or serine at amino acid position 156 of SEQ ID NO:20; (d) a threonine or serine at amino acid position 159 of SEQ ID NO:20; (e) a lysine, arginine or histidine at amino acid position 170 of SEQ ID NO:20; (f) a glutamic acid or aspartic acid at amino acid position 198 of SEQ ID NO:20; and (g) an alanine, glycine, isoleucine, leucine or valine amino acid position 252 of SEQ ID NO:20. In other aspects, in addition to the above mutations (a)-(g), the protein switch further comprises one or more of the following mutations: (1) a glycine, isoleucine, leucine or valine at amino acid position 11 of SEQ ID NO:20; (2) a glycine, isoleucine, leucine or valine at amino acid position 22 of SEQ ID NO:20; (3) an asparagine or glutamine at amino acid position 45 of SEQ ID NO:20; (4) an alanine, glycine, isoleucine or leucine at amino acid position 48 of SEQ ID NO:20; (5) an aspartic acid or glutamic acid at amino acid position 55 of SEQ ID NO:20; (6) an alanine, glycine, isoleucine, leucine or valine at amino acid position 98 of SEQ ID NO:20; (7) a phenylalanine, tryptophan, or tyrosine at amino acid position 137 of SEQ ID NO:20; (8) an alanine, glycine, leucine or valine at amino acid position 141 of SEQ ID NO:20; (9) an alanine, glycine, isoleucine or leucine at amino acid position 149 of SEQ ID NO:20; (10) an alanine, glycine, isoleucine or valine at amino acid position 154 of SEQ ID NO:20; (11) a threonine or serine at amino acid position 166 SEQ ID NO:20; (12) a glycine, isoleucine, leucine or valine at amino acid position 173 SEQ ID NO:20; (13) a threonine or serine at amino acid position 184 of SEQ ID NO:20; (14) a histidine, leucine, or arginine at amino acid position 195 of SEQ ID NO:20; (15) a threonine or serine at amino acid position 219 of SEQ ID NO:20; (16) an asparagine or glutamine at amino acid position 240 of SEQ ID NO:20; and/or (17) an alanine, glycine, isoleucine, leucine or valine at amino acid position 251 at SEQ ID NO:20.

In yet another aspect, the protein switch comprises 7 mutations at amino acid position 96, 155, 156, 159, 170, 198 and 252 of SEQ ID NO:20. In these aspects, the protein switch comprises a cysteine or a phenylalanine at amino acid position 96, an alanine at amino acid position 155, a serine at amino acid position 156, a tyrosine at amino acid position 159, a lysine at amino acid position 170, a glutamic acid at amino acid position 198, and a lysine or valine at amino acid position 252 of SEQ ID NO:20.

In another aspect, the protein switch comprises 8 mutations at amino acid position 96, 98, 155, 156, 159, 170, 198 and 252 of SEQ ID NO:20. In these aspects, the protein switch comprises a cysteine or a phenylalanine at amino acid position 96, a leucine at amino acid position 98, an alanine at amino acid position 155, a serine at amino acid position 156, a tyrosine at amino acid position 159, a lysine at amino acid position 170, a glutamic acid at amino acid position 198, and a lysine or valine at amino acid position 252 of SEQ ID NO:20.

In another aspect, the protein switch comprises 8 mutations at amino acid position 96, 155, 156, 159, 170, 198, 219 and 252 of SEQ ID NO:20. In these aspects, the protein switch comprises a cysteine or a phenylalanine at amino acid position 96, an alanine at amino acid position 155, a serine at amino acid position 156, a tyrosine at amino acid position 159, a lysine at amino acid position 170, a glutamic acid at amino acid position 198, a threonine at amino acid position 219, and a lysine or valine at amino acid position 252 of SEQ ID NO:20.

In another aspect, the protein switch comprises 8 mutations at amino acid position 96, 155, 156, 159, 170, 195, 198 and 252 of SEQ ID NO:20. In these aspects, the protein switch comprises a cysteine or a phenylalanine at amino acid position 96, an alanine at amino acid position 155, a serine at amino acid position 156, a tyrosine at amino acid position 159, a lysine at amino acid position 170, an arginine at amino acid position 195, a glutamic acid at amino acid position 198, and a lysine or valine at amino acid position 252 of SEQ ID NO:20.

In another aspect, the protein switch comprises 8 mutations at amino acid position 96, 141, 155, 156, 159, 170, 198 and 252 of SEQ ID NO:20. In these aspects, the protein switch comprises a cysteine or a phenylalanine at amino acid position 96, a valine at amino acid position 141, an alanine at amino acid position 155, a serine at amino acid position 156, a tyrosine at amino acid position 159, a lysine at amino acid position 170, a glutamic acid at amino acid position 198, and a lysine or valine at amino acid position 252 of SEQ ID NO:20.

In another aspect, the protein switch comprises 8 mutations at amino acid position 96, 155, 156, 159, 170, 173, 198 and 252 of SEQ ID NO:20. In these aspects, the protein switch comprises a cysteine or a phenylalanine at amino acid position 96, an alanine at amino acid position 155, a serine at amino acid position 156, a tyrosine at amino acid position 159, a lysine at amino acid position 170, a glycine at amino acid position 173, a glutamic acid at amino acid position 198, and a lysine or valine at amino acid position 252 of SEQ ID NO:20.

In another aspect, the protein switch comprises 8 mutations at amino acid position 55, 96, 155, 156, 159, 170, 198 and 252 of SEQ ID NO:20. In these aspects, the protein switch comprises a glutamic acid at amino acid position 55, a cysteine or a phenylalanine at amino acid position 96, an alanine at amino acid position 155, a serine at amino acid position 156, a tyrosine at amino acid position 159, a lysine at amino acid position 170, a glutamic acid at amino acid position 198, and a lysine or valine at amino acid position 252 of SEQ ID NO:20.

In another aspect, the protein switch comprises 8 mutations at amino acid position 96, 137, 155, 156, 159, 170, 198 and 252 of SEQ ID NO:20. In these aspects, the protein switch comprises a cysteine or a phenylalanine at amino acid position 96, a tyrosine at amino acid position 137, an alanine at amino acid position 155, a serine at amino acid position 156, a tyrosine at amino acid position 159, a lysine at amino acid position 170, a glutamic acid at amino acid position 198, and a lysine or valine at amino acid position 252 of SEQ ID NO:20.

In another aspect, the protein switch comprises 8 mutations at amino acid position 48, 96, 155, 156, 159, 170, 198 and 252 of SEQ ID NO:20. In these aspects, the protein switch comprises an alanine at amino acid position 48, a cysteine or a phenylalanine at amino acid position 96, an alanine at amino acid position 155, a serine at amino acid position 156, a tyrosine at amino acid position 159, a lysine at amino acid position 170, a glutamic acid at amino acid position 198, and a lysine or valine at amino acid position 252 of SEQ ID NO:20.

In another aspect, the protein switch comprises 8 mutations at amino acid position 96, 155, 156, 159, 170, 184, 198 and 252 of SEQ ID NO:20. In these aspects, the protein switch comprises a cysteine or a phenylalanine at amino acid position 96, an alanine at amino acid position 155, a serine at amino acid position 156, a tyrosine at amino acid position 159, a lysine at amino acid position 170, a serine at amino acid position 184, a glutamic acid at amino acid position 198, and a lysine or valine at amino acid position 252 of SEQ ID NO:20.

In another aspect, the protein switch comprises 8 mutations at amino acid position 96, 155, 156, 159, 166, 170, 198 and 252 of SEQ ID NO:20. In these aspects, the protein switch comprises a cysteine or a phenylalanine at amino acid position 96, an alanine at amino acid position 155, a serine at amino acid position 156, a tyrosine at amino acid position 159, a serine at amino acid position 166, a lysine at amino acid position 170, a glutamic acid at amino acid position 198, and a lysine or valine at amino acid position 252 of SEQ ID NO:20.

In another aspect, the protein switch comprises 8 mutations at amino acid position 96, 155, 156, 159, 170, 198, 240 and 252 of SEQ ID NO:20. In these aspects, the protein switch comprises a cysteine or a phenylalanine at amino acid position 96, an alanine at amino acid position 155, a serine at amino acid position 156, a tyrosine at amino acid position 159, a lysine at amino acid position 170, a glutamic acid at amino acid position 198, a glutamine at amino acid position 240, and a lysine or valine at amino acid position 252 of SEQ ID NO:20.

In still yet another aspect, the protein switch comprises 9 mutations at amino acid position 45, 96, 149, 155, 156, 159, 170, 198 and 252 of SEQ ID NO:20. In these aspects, the protein switch comprises an asparagine at amino acid position 45, a cysteine or a phenylalanine at amino acid position 96, an alanine at amino acid position 149, an alanine at amino acid position 155, a serine at amino acid position 156, a tyrosine at amino acid position 159, a lysine at amino acid position 170, a glutamic acid at amino acid position 198, and a lysine or valine at amino acid position 252 of SEQ ID NO:20.

In still yet another aspect, the protein switch comprises 9 mutations at amino acid position 22, 96, 154, 155, 156, 159, 170, 198 and 252 of SEQ ID NO:20. In these aspects, the protein switch comprises a glycine at amino acid position 22, a cysteine or a phenylalanine at amino acid position 96, a glycine at amino acid position 154, an alanine at amino acid position 155, a serine at amino acid position 156, a tyrosine at amino acid position 159, a lysine at amino acid position 170, a glutamic acid at amino acid position 198, and a lysine or valine at amino acid position 252 of SEQ ID NO:20.

In yet another aspect, the protein switch comprises 9 mutations at amino acid position 96, 141, 154, 155, 156, 159, 170, 198 and 252 of SEQ ID NO:20. In these aspects, the protein switch comprises a cysteine or a phenylalanine at amino acid position 96, a valine at amino acid position 141, a glycine at amino acid position 154, an alanine at amino acid position 155, a serine at amino acid position 156, a tyrosine at amino acid position 159, a lysine at amino acid position 170, a glutamic acid at amino acid position 198, and a lysine or valine at amino acid position 252 of SEQ ID NO:20.

In still yet a further aspect, the protein switch comprises 10 mutations at amino acid position 12, 96, 155, 156, 159, 170, 195, 198, 251 and 252 of SEQ ID NO:20. In these aspects, the protein switch comprises a lysine at amino acid position 12, a cysteine or a phenylalanine at amino acid position 96, an alanine at amino acid position 155, a serine at amino acid position 156, a tyrosine at amino acid position 159, a lysine at amino acid position 170, an arginine at amino acid position 195, a glutamic acid at amino acid position 198, a lysine at amino acid position 251, and a lysine or valine at amino acid position 252 of SEQ ID NO:20.

In another embodiment, the present disclosure relates to a protein switch comprising an amino acid sequence having at least 80% identity to SEQ ID NO:11.

In one aspect, the protein switch comprises an amino acid sequence having at least 85% identity to SEQ ID NO:11.

In yet another aspect, the protein switch comprises an amino acid sequence having at least 90% identity to SEQ ID NO:11.

In still yet another aspect, the protein switch comprises an amino acid sequence having at least 95% identity to SEQ ID NO:11.

In still yet another aspect, the protein switch comprises an amino acid sequence having at least 96% identity to SEQ ID NO:11.

In still yet another aspect, the protein switch comprises an amino acid sequence having at least 97% identity to SEQ ID NO:11.

In still yet another aspect, the protein switch comprises an amino acid sequence having at least 98% identity to SEQ ID NO:11.

In still yet another aspect, the protein switch comprises an amino acid sequence having at least 99% identity to SEQ ID NO:11.

In still yet another aspect, the protein switch comprises an amino acid sequence having at least 100% identity to SEQ ID NO:11.

In still yet another aspect, the protein switch comprises an amino acid sequence of any one of SEQ ID NOS. 1-10 or 12-19.

In yet another embodiment, the present disclosure relates to a composition or kit comprising at least one of the protein switches described above and at least one reactant. In one aspect, the reactant in the composition or kit is glucose or lactate.

In yet another embodiment, the present disclosure relates to a method of detecting an analyte. The method comprises the steps of:
a. providing a protein switch comprising at least one polypeptide having (a) at least one analyte binding domain capable of binding with at least one analyte; and (b) at least one oxidase or dehydrogenase domain having oxidase or dehydrogenase activity and capable of binding or reacting with at least one reactant, wherein (i) the analyte that binds to the analyte binding domain is different than the reactant that binds or reacts with the oxidase or dehydrogenase domain; and (ii) when the analyte binds to the analyte binding domain the oxidase or dehydrogenase activity changes;
b. contacting the protein switch with a fluid comprising a reactant specific to the protein switch, wherein the analyte binding domain binds to the analyte in the fluid whereby the oxidase or dehydrogenase activity changes; and
c. detecting a change in a rate of breakdown of the reactant by the oxidase or dehydrogenase domain of the protein switch.

In one aspect of the above method, the oxidase is glucose oxidase or lactate oxidase.

In another aspect of the above method, the dehydrogenase is glucose dehydrogenase or lactate dehydrogenase.

In still yet another aspect of the above method, the reactant is glucose or lactate.

In still yet another aspect of the above method, the analyte is warfarin, cortisol, methotrexate, or triiodothyronine.

In yet another embodiment, the present disclosure relates to a method of detecting an analyte. The method comprises the steps of:
a. providing one of the above described protein switches;
b. contacting the protein switch with a fluid comprising a reactant specific to the protein switch, wherein the analyte binding domain binds to the analyte in the fluid whereby the oxidase or dehydrogenase activity changes; and
c. detecting a change in a rate of breakdown of the reactant by the oxidase or dehydrogenase domain of the protein switch.

In one aspect of the above method, the protein switch comprises an amino acid sequence having at least 80% identity to SEQ ID NO:11.

In one aspect of the above method, the protein switch comprises an amino acid sequence having at least 85% identity to SEQ ID NO:11.

In another aspect of the above method, the protein switch comprises an amino acid sequence having at least 90% identity to SEQ ID NO:11.

In yet another aspect of the above method, the protein switch comprises an amino acid sequence having at least 95% identity to SEQ ID NO:11.

In still yet another aspect of the above method, the protein switch comprises an amino acid sequence having at least 96% identity to SEQ ID NO:11.

In still yet another aspect of the above method, the protein switch comprises an amino acid sequence having at least 97% identity to SEQ ID NO:11.

In still yet another aspect of the above method, the protein switch comprises an amino acid sequence having at least 98% identity to SEQ ID NO:11.

In still yet another aspect of the above method, the protein switch comprises an amino acid sequence having at least 99% identity to SEQ ID NO:11.

In still yet another aspect of the above method, the protein switch comprises an amino acid sequence having at least 100% identity to SEQ ID NO:11.

In still yet another aspect of the above method, the protein switch comprises an amino acid sequence of any one of SEQ ID NOS. 1-10 or 12-19.

In still yet another aspect of the above method, the oxidase is glucose oxidase or lactate oxidase.

In still yet another aspect of the above method, the dehydrogenase is glucose dehydrogenase or lactate dehydrogenase.

In still yet another aspect of the above method, the reactant is glucose or lactate.

In still yet another aspect of the above method, the analyte is warfarin, cortisol, methotrexate, or triiodothyronine.

In yet another embodiment, the present disclosure relates to a system for detecting or monitoring an analyte concentration. The system comprises a sensor control device and a signal detecting device, wherein the sensor control device comprises at least one sensor comprising one of the protein switches described above.

In one aspect, the oxidase in the protein switch used in the sensor is glucose oxidase or lactate oxidase.

In another aspect, the dehydrogenase in the protein switch used in the sensor glucose dehydrogenase, or lactate dehydrogenase.

In yet another embodiment, the present disclosure relates to an analyte monitoring system. The analyte monitoring system comprises a sensor comprising a substrate, one or more working electrodes, and one of the protein switches described above, at least a portion of the sensor being adapted for implantation and intimate contact with bodily fluid, the sensor being configured and arranged to produce a signal representative of a level of an analyte in the bodily fluid; and a signal detecting device for receiving the signal, wherein said signal is generated by contact of said analyte with said protein switch.

In one aspect, the oxidase in the protein switch used in the analyte monitoring system is glucose oxidase or lactate oxidase.

In another aspect, the dehydrogenase in the protein switch used in the analyte monitoring system is glucose dehydrogenase or lactate dehydrogenase.

In yet a further aspect, the analyte detected in the analyte monitoring system is warfarin, cortisol, methotrexate, or triiodothyronine.

### BRIEF DESCRIPTION OF THE FIGURES

FIG. 1 shows a diagram of an illustrative sensing system that incorporates a sensor comprising at least one protein switch of the present disclosure.

FIG. 2A shows a diagram of an illustrative two-electrode sensor configuration having a single working electrode. FIGS. 2B and 2C show diagrams of illustrative three-electrode sensor configurations having a single working electrode.

FIG. 3 shows a diagram of an illustrative sensor configuration having two working electrodes, a reference electrode and a counter electrode.

FIG. 4 shows an illustrative sensor configuration in which two different active areas are disposed upon the surface of a single working electrode.

FIGS. 5A and 5B show diagrams of illustrative working electrodes in which a first active area is disposed directly upon a surface of the working electrode and a second active area is separated from the working electrode by a membrane.

FIG. 6 shows an illustrative schematic of a portion of a sensor having two working electrodes and featuring a bilayer membrane overcoating one of the two working electrodes.

FIG. 7 shows the linear sensitivity response of sensors comprising the protein switches GDH-2004, GDH-2007, GDH-2008, GDH-2009, GDH-2010, GDH-2011, GDH-2015, GDH-2018, GDH-2019, GDH-2005, GDH-2013, GDH-2016, and GDH-2024 to increasing glucose concentrations. The sensor comprising GDH-105 is the control.

FIG. 8 shows the beaker (long-term) stability of sensors comprising the protein switches GDH-2004, GDH-2007, GDH-2008, GDH-2009, GDH-2010, GDH-2011, GDH-2015, GDH-2018, GDH-2019, GDH-2005, GDH-2013, GDH-2016, and GDH-2024. The sensor comprising GDH-105 is the control.

FIGS. 9A and 9B show the inhibition of a sensor comprising protein switch GDH-2016 to increasing warfarin concentrations while the sensor comprising GDH-105 (control) was not affected.

FIG. 10 shows the linear sensitivity response of the sensor comprising GDH-105 (control) and sensors comprising protein switches GDH-2025, GDH-2026, GDH-2027 and GDH-2028 based on beaker calibration.

FIG. 11 shows the beaker (long-term) stability of sensors comprising the protein switches GDH-2025, GDH-2026, GDH-2027 and GDH-2028. The sensor comprising GDH-105 is the control.

FIGS. 12A and 12B shows the inhibition of sensors comprising protein switches GDH-2016, GDH-2025, GDH-2026, GDH-2027 and GDH-20288 to increasing warfarin concentrations. The sensor comprising GDH-105 is the control.

FIG. 13 shows the inhibition of sensors comprising GDH-2016, GDH-2025, GDH-2026, GDH-2027 and GDH-20288 to increasing warfarin concentrations. The sensor comprising GDH-105 is the control.

FIG. 14 shows the sequences of SEQ ID NOS. 1-20.

### DETAILED DESCRIPTION

Provided herein are example aspects of compositions of matter, methods, devices (e.g., sensors), systems, and apparatus for detecting and/or monitoring target analytes in a bodily fluid or biological sample. It is to be understood that the teachings of this disclosure are not limited to the particular aspects described, and as such can, of course, vary. It is also to be understood that the terminology used herein is for the purpose of describing particular aspects only and is not intended to be limiting.

For purposes of illustration, and not limitation, protein switches can be designed to recognize, identify and/or quantify one or more target analytes in a bodily fluid or biological sample. The exact nature and configuration of the protein switch can vary. A protein switch can have an analyte binding part for detection of the target analyte and an enzyme part that generates a product (e.g., a signal) or change in the product generated (e.g., change in a signal) upon binding of an analyte. Protein switches can be utilized in a variety of conditions and configurations, including in a sensor for measuring the levels of the analyte in a subject. The configuration of such a sensor can depend on the analyte measured and the bodily fluid which the device monitors for the analyte. The sensor can be configured for detecting and/or measuring analyte in vivo in a subject. In vivo sensors can include an insertion tip positionable below the surface of the skin, e.g., penetrating through the skin and into the dermis or the subcutaneous region. The sensor can test for analyte in the dermal fluid, interstitial fluid, subcutaneous fluid, or blood (e.g., capillary).

The sensor can include one or more protein switches that binds an analyte (in the analyte binding part or portion) and then generates a product (such as a signal from the enzyme part or portion) that can be detected by the sensor. Protein switches in a sensor can have one level of activity, (e.g., lower or higher) when analyte is not bound and a different activity (e.g., higher or lower), when analyte is bound. For example, analyte binding by the analyte binding portion of the protein switch can reduce enzyme activity. Alternatively, analyte binding by the analyte binding portion of the protein switch can increase enzyme activity. The change in enzyme activity can be detected (directly or indirectly) by the sensor as a change in signal based on the amount of product made by the enzyme. The signal detected by the sensor can be correlated to the amount of analyte which can be prognostic or diagnostic for a patient's health and/or well-being. Alternatively, the signal detected at the sensor can be correlated to the amount of analyte which is used to monitor a condition of a patient's health and/or well-being. The amount of analyte measured can also be correlated and/or converted to amounts in the blood or other bodily fluids.

### A. Definitions

Unless otherwise defined, all technical and scientific terms used herein have the same meaning as commonly understood by one of ordinary skill in the art. In case of conflict, the present document, including definitions, will control. Preferred methods and materials are described below, although methods and materials similar or equivalent to those described herein can be used in practice or testing of the present disclosure. All publications, patent applications, patents and other references mentioned herein are incorporated by reference in their entirety. The materials, methods, and examples disclosed herein are illustrative only and not intended to be limiting.

The terms “comprise(s),” “include(s),” “having,” “has,” “can,” “contain(s),” and variants thereof, as used herein, are intended to be open-ended transitional phrases, terms, or words that do not preclude the possibility of additional acts or structures. The singular forms “a,” “and” and “the” include plural references unless the context clearly dictates otherwise. The present disclosure also contemplates other aspects “comprising,” “consisting of” and “consisting essentially of,” the aspects or elements presented herein, whether explicitly set forth or not.

For the recitation of numeric ranges herein, each intervening number there between with the same degree of precision is explicitly contemplated. For example, for the range of 6-9, the numbers 7 and 8 are contemplated in addition to 6 and 9, and for the range 6.0-7.0, the number 6.0, 6.1, 6.2, 6.3, 6.4, 6.5, 6.6, 6.7, 6.8, 6.9, and 7.0 are explicitly contemplated.

“Affimer” as used herein generally refers to peptides that specifically or selectively bind to a target (e.g., analyte, target tissue, target molecule, target cell, etc.). Generally, affimers can be small peptides or proteins, generally with a molecular weight less than 12 kDa. Affimers can have the capacity to recognize specific epitopes or antigens, and with binding affinities that can be close to those of antibodies (e.g., in the low nanomolar to picomolar range); however, the term “affirmer,” as used herein, does not encompass antibodies, immunoglobulins, Fab regions of antibodies, or Fc regions of antibodies. Affimers can have the same specificity advantage of antibodies, but can be smaller, can be chemically synthesized or chemically modified, and have the advantage of being free from cell culture contaminants.

“Sensor”, “in vivo sensor,” or “sensor” as used interchangeably herein refers to a device configured to detect the presence and/or measure the level of one or more (e.g., multiple) analytes in a sample via electrochemical oxidation and reduction reactions on the sensor. These reactions are transduced to an electrical signal that can be correlated to an amount, concentration, or level of an analyte or activity of an enzyme in the sample.

“Enzyme” refers to a protein or a fragment thereof having activity (e.g., catalytic activity, enzyme activity or enzymatic activity) towards one or more reactants (e.g., enzyme substrate). Examples of one or more reactants (e.g., enzyme substrates) are glucose, lactate, glutamate, ascorbic acid, cholesterol, choline acetylcholine, hypoxanthine, norepinephrine, 5-hydroxytryptamine, phenylethylamine and e/e-methylhistamine, a polyphenol, ethanol, an aldehyde, or malate.

“Fluid,” “bodily fluid,” “sample,” or “biological sample” as used interchangeably herein, refers to dermal fluid, interstitial fluid, subcutaneous fluid, or blood (e.g. such as capillary) obtained from a subject or patient. In one aspect, the fluid or sample is dermal fluid. In one aspect, the fluid or sample is interstitial fluid. In yet another aspect, the fluid is subcutaneous fluid. In yet another aspect, the fluid or sample is blood (such as capillary).

“Identical”, “identity,” or “sequence identity” as used herein in the context of two or more polypeptide or polynucleotide sequences, can mean that the sequences have a specified percentage of residues that are the same over a specified region that is determined using a standard algorithm, having regard to the extent that sequences are identical over a window of comparison. Identity or sequence identity may be determined using computer algorithms such as GAP, BESTFIT, FASTA and the BLAST family of programs as for example disclosed by Altschul et al., 1997, Nucl. Acids Res. 25 3389. A detailed discussion of sequence analysis can be found in Unit 19.3 of CURRENT PROTOCOLS IN MOLECULAR BIOLOGY Eds. Ausubel et al. (John Wiley & Sons Inc NY, 1995-1999). Suitably, sequence identity is measured over the entire length of SEQ ID NOS. 1-20.

“Isolated polynucleotide” as used herein may mean a polynucleotide (e.g., of genomic, cDNA, or synthetic origin, or a combination thereof) that, by virtue of its origin, the isolated polynucleotide is not associated with all or a portion of a polynucleotide with which the “isolated polynucleotide” is found in nature.

“Sensing layer” as used herein refers to a component of the sensor which includes constituents that facilitate electrooxidation or electroreduction of a compound either directly at an electrode or via one or more electron transfer agents, one or more co-factors or a combination of one or more electron transfer agents and one or more co-factors. In some aspects of the sensor, the sensing layer is disposed in proximity to or on the working electrode.

“Sensing region” as used herein refers to the active chemical area of a sensor.

“Subject” or “patient” as used herein interchangeably refers to any vertebrate, including, but not limited to, a mammal (e.g., cow, pig, camel, llama, horse, goat, rabbit, sheep, hamsters, guinea pig, cat, dog, rat, and mouse, a non-human primate (for example, a monkey, such as a cynomolgus or rhesus monkey, chimpanzee, etc.) and a human). In some aspects, the subject may be a human or a non-human. In some aspects, the subject is a human. The subject or patient may be undergoing one or more forms of treatment.

“The term “variant protein”, “protein variant”, or “variant” as used interchangeably herein is meant a protein that differs from that of a parent protein by virtue of at least one amino acid modification. The term “protein variant” may refer to the protein itself, a composition comprising the protein, or the amino sequence that encodes it. In some aspects, the protein variant has at least one amino acid modification compared to the parent or reference protein, e.g. from about one to about fifty amino acid modifications compared to the parent protein. In some aspects, the protein variant has from about one to about forty amino acid modifications compared to the parent protein. In some aspects, the protein variant has from about one to about thirty amino acid modifications compared to the parent protein. In some aspects, the protein variant has from about one to about twenty amino acid modifications compared to the parent protein. In some aspects, the protein variant has from about one to about ten amino acid modifications compared to the parent protein. In some aspects, the protein variant has from about one to about five amino acid modifications compared to the parent protein. In some aspects, a protein variant sequence herein will possess at least about 80% identity with a parent protein sequence. In other aspects, a protein variant sequence herein will possess at least about 90% identity. In still other aspects, a protein variant sequence will possess least about 95%, 96%, 97%, 98%, or 99% identity.

In the present disclosure, reference is made to amino acids. In addition to the name of amino acids, the three-letter and one-letter codes are also used herein. For clarity purposes, the amino acids referred to in this disclosure are referred to as follows: alanine (Ala, A), arginine (Arg, R), asparagine (Asn, N), Aspartic acid (Asp, D), cysteine (Cys, C), glutamine (Qln, Q), glutamic acid (Glu, E), glycine (Gly, G), histidine (His, H), isoleucine (Ile, I), leucine (Leu, L), lysine (Lys, K), methionine (Met, M), phenylalanine (Phe, F), proline (Pro, P), serine (Ser, S), threonine (Thr, T), tryptophan (Trp, W), tyrosine (Tyr, Y) and valine (Val, V).

Unless otherwise defined herein, scientific and technical terms used in connection with the present disclosure shall have the meanings that are commonly understood by those of ordinary skill in the art. For example, any nomenclatures used in connection with, and techniques of, cell and tissue culture, molecular biology, immunology, microbiology, genetics and protein and nucleic acid chemistry and hybridization described herein are those that are well known and commonly used in the art. The meaning and scope of the terms should be clear; in the event, however of any latent ambiguity, definitions provided herein take precedent over any dictionary or extrinsic definition. Further, unless otherwise required by context, singular terms shall include pluralities and plural terms shall include the singular.

### B. Protein Switches

In some aspects, the present disclosure relates to compositions of matter which can comprise one or more protein switches. Specifically, a protein switch can include, but is not limited to, at least two parts: at least one analyte binding part (or portion) and at least one enzyme part (or portion). The analyte binding part modulates the activity of the enzyme part of the protein switch. As will be discussed in further detail herein, when an analyte binds to the analyte binding part, the activity of the enzyme part changes (e.g., increases or decreases), as a result of, for example, competitive inhibition, uncompetitive inhibition or non-competitive inhibition. In some aspects, when an analyte binds to the analyte binding part, the activity of the enzyme part increases (when compared to without the analyte). In other aspects, when an analyte binds to the analyte binding part, the activity of the enzyme part decreases (when compared to without the analyte). Such modulation of enzyme activity can provide for detection of the analyte.

The protein switch of the present disclosure comprises at least one non-naturally occurring polypeptide having at least two different domains or portions. In some aspects, the at least two different domains or portions may overlap with each other either in terms of their sequence (e.g., contain one or more nucleic acids or amino acid sequences which overlap) or in their spatial orientation. The first domain or portion is the analyte binding part (or binding portion) which comprises at least one analyte binding domain. The at least one analyte binding domain binds or is capable of binding with one or more analytes of interest. The second domain or portion is the enzyme part which comprises at least one oxidase or dehydrogenase domain having enzyme activity (e.g., catalytic activity). In other words, the second domain or portion is catalytically active. Additionally, the oxidase or dehydrogenase domain binds, reacts, is capable of binding and/or is capable of reacting with one or more reactants and optionally, one or more cofactors to generate or produce a product (e.g., a signal) that can be detected, which will be discussed in more detail herein.

In one aspect, in the presence of analyte (e.g., when at least one analyte binds to an analyte binding domain), the oxidase or dehydrogenase domain binds and/or reacts with at least one reactant and the activity of the enzyme (e.g., oxidase or dehydrogenase) increases. Specifically, the amount of the product (e.g., signal) produced increases. The activity of the enzyme may increase (when compared to the wildtype enzyme) by about 0.1%, about 0.5%, about 1%, about 2%, about 3%, about 4%, about 5%, about 6%, about 7%, about 8%, about 9%, about 10%, about 11%, about 12%, about 13%, about 14%, about 15%, about 16%, about 17%, about 18%, about 19%, about 20%, about 21%, about 22%, about 23%, about 24%, about 25%, about 26%, about 27%, about 28%, about 29%, about 30%, about 40%, about 41%, about 42%, about 43%, about 44%, about 45%, about 46%, about 47%, about 48%, about 49%, about 50%, about 51%, about 52%, about 53%, about 54%, about 55%, about 56%, about 57%, about 58%, about 59%, about 60%, about 61%, about 62%, about 63%, about 64%, about 65%, about 66%, about 67%, about 68%, about 69%, about 70%, about 71%, about 72%, about 73%, about 74%, about 75%, about 76%, about 77%, about 78%, about 79%, about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, about 99% or about 100%.

In still yet another aspect, in the presence of analyte (e.g., when the analyte binding region binds to one or more analytes), the oxidase or dehydrogenase domain does not bind, or react or exhibits reduced (e.g., inhibited) binding and/or reaction to one or more reactants and the activity of the enzyme (e.g., oxidase or dehydrogenase) may decrease. Specifically, the amount of the product (e.g., signal) produced decreases. The activity of the enzyme (e.g., oxidase or dehydrogenase) may decrease (when compared to the wildtype enzyme) by about 0.1%, about 0.5%, about 1%, about 2%, about 3%, about 4%, about 5%, about 6%, about 7%, about 8%, about 9%, about 10%, about 11%, about 12%, about 13%, about 14%, about 15%, about 16%, about 17%, about 18%, about 19%, about 20%, about 21%, about 22%, about 23%, about 24%, about 25%, about 26%, about 27%, about 28%, about 29%, about 30%, about 40%, about 41%, about 42%, about 43%, about 44%, about 45%, about 46%, about 47%, about 48%, about 49%, about 50%, about 51%, about 52%, about 53%, about 54%, about 55%, about 56%, about 57%, about 58%, about 59%, about 60%, about 61%, about 62%, about 63%, about 64%, about 65%, about 66%, about 67%, about 68%, about 69%, about 70%, about 71%, about 72%, about 73%, about 74%, about 75%, about 76%, about 77%, about 78%, about 79%, about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, about 99% or about 100% (e.g., complete inhibition of the activity of the enzyme (e.g., oxidase or dehydrogenase)).

In one aspect, the analyte binding domain can be derived from any protein or polypeptide that binds or is capable of binding to one or more analytes of interest. For example, in one aspect, the analyte binding domain can be derived from one or more affimers, antibodies, peptides, receptors (including full length, and single chain receptors), small molecules, artificial binding proteins (such as those made using scaffolds, display technologies, etc.) or any functional fragments, or variants thereof. In another aspect, the analyte binding domain can be derived from a polypeptide or protein (such as an oxidase or dehydrogenase) that has been mutated and/or modified such as to contain one or more amino acid mutations, deletions, substitutions and/or truncations. In some aspects, the binding affinity of the analyte binding domain for an analyte allows the protein switch to detect one or more analytes at physiological levels.

The length of the analyte binding domain is not critical, provided that the analyte binding domain binds or is capable of binding to one or more analytes of interest. For example, the analyte binding domain can have a length of 3 consecutive amino acids, 4 consecutive amino acids, 5 consecutive amino acids, 6 consecutive amino acids, 7 consecutive amino acids, 8 consecutive amino acids, 9 consecutive amino acids, 10 consecutive amino acids, 11 consecutive amino acids, 12 consecutive amino acids, 13 consecutive amino acids, 14 consecutive amino acids, 15 consecutive amino acids, 16 consecutive amino acids, 17 consecutive amino acids, 18 consecutive amino acids, 19 consecutive amino acids, 20 consecutive amino acids, 21 consecutive amino acids, 22 consecutive amino acids, 23 consecutive amino acids, 24 consecutive amino acids, 25 consecutive amino acids, 26 consecutive amino acids, 27 consecutive amino acids, 28 consecutive amino acids, 29 consecutive amino acids, 30 consecutive amino acids, 31 consecutive amino acids, 32 consecutive amino acids, 33 consecutive amino acids, 34 consecutive amino acids, 35 consecutive amino acids, 36 consecutive amino acids, 37 consecutive amino acids, 38 consecutive amino acids, 39 consecutive amino acids, 40 consecutive amino acids, 41 consecutive amino acids, 42 consecutive amino acids, 43 consecutive amino acids, 44 consecutive amino acids, 45 consecutive amino acids, 46 consecutive amino acids, 47 consecutive amino acids, 48 consecutive amino acids, 49 consecutive amino acids, 50 consecutive amino acids, consecutive amino acids, 75 consecutive amino acids, 80 consecutive amino acids, 85 consecutive amino acids, 90 consecutive amino acids, 95 consecutive amino acids, 100 consecutive amino acids, 125 consecutive amino acids, 150 consecutive amino acids, 175 consecutive amino acids, 200 consecutive amino acids, 225 consecutive amino acids, 250 consecutive amino acids, 275 consecutive amino acids, 300 consecutive amino acids, 325 consecutive amino acids, 350 consecutive amino acids, 375 consecutive amino acids, 400 consecutive amino acids, 425 consecutive amino acids, 450 consecutive amino acids, 475 consecutive amino acids, or 500 consecutive amino acids.

In another aspect, the at least one oxidase or dehydrogenase domain or portion comprises an amino acid sequence that encodes at least one oxidase and/or at least one dehydrogenase having enzyme activity (e.g. catalytic activity) and binds, reacts, is capable of binding and/or reacting with at least one reactant and optionally, a cofactor to produce a product (e.g. a signal). The amino acid sequence encoding the oxidase or dehydrogenase can be the native (wildtype) sequence derived, obtained and/or synthesized from one or more microorganisms, such as from a bacteria, virus, or fungus, or mammalian cell. The amino acid sequence can encode the entire enzyme or a functional fragment thereof (provided that the functional fragment has enzyme activity). Alternatively, the amino acid sequence of the oxidase or dehydrogenase domain can be a variant of the native (wildtype) sequence that encodes an oxidase or dehydrogenase having enzyme activity. As with the analyte binding domain, the length of the oxidase or dehydrogenase domain is not critical provided that it encodes an enzyme (e.g., entire enzyme), functional fragment, or variant thereof having enzyme (e.g., oxidase or dehydrogenase) activity. For example, the oxidase or dehydrogenase domain can have a length of 3 consecutive amino acids, 4 consecutive amino acids, 5 consecutive amino acids, 6 consecutive amino acids, 7 consecutive amino acids, 8 consecutive amino acids, 9 consecutive amino acids, 10 consecutive amino acids, 11 consecutive amino acids, 12 consecutive amino acids, 13 consecutive amino acids, 14 consecutive amino acids, 15 consecutive amino acids, 16 consecutive amino acids, 17 consecutive amino acids, 18 consecutive amino acids, 19 consecutive amino acids, 20 consecutive amino acids, 21 consecutive amino acids, 22 consecutive amino acids, 23 consecutive amino acids, 24 consecutive amino acids, 25 consecutive amino acids, 26 consecutive amino acids, 27 consecutive amino acids, 28 consecutive amino acids, 29 consecutive amino acids, 30 consecutive amino acids, 31 consecutive amino acids, 32 consecutive amino acids, 33 consecutive amino acids, 34 consecutive amino acids, 35 consecutive amino acids, 36 consecutive amino acids, 37 consecutive amino acids, 38 consecutive amino acids, 39 consecutive amino acids, 40 consecutive amino acids, 41 consecutive amino acids, 42 consecutive amino acids, 43 consecutive amino acids, 44 consecutive amino acids, 45 consecutive amino acids, 46 consecutive amino acids, 47 consecutive amino acids, 48 consecutive amino acids, 49 consecutive amino acids, 50 consecutive amino acids, consecutive amino acids, 75 consecutive amino acids, 80 consecutive amino acids, 85 consecutive amino acids, 90 consecutive amino acids, 95 consecutive amino acids, 100 consecutive amino acids, 125 consecutive amino acids, 150 consecutive amino acids, 175 consecutive amino acids, 200 consecutive amino acids, 225 consecutive amino acids, 250 consecutive amino acids, 275 consecutive amino acids, 300 consecutive amino acids, 325 consecutive amino acids, 350 consecutive amino acids, 375 consecutive amino acids, 400 consecutive amino acids, 425 consecutive amino acids, 450 consecutive amino acids, 475 consecutive amino acids, or 500 consecutive amino acids.

Enzymes which can be used in the at least one oxidase or dehydrogenase domain include (i) one or more oxidases, such as, for example, glucose oxidase or lactate oxidase; (ii) one or more dehydrogenases, such as, for example, glucose dehydrogenase or lactate dehydrogenase; or (iii) any combinations of (i) and (ii). In some aspects, the oxidase is glucose oxidase. In other aspects, the oxidase is lactate oxidase. In some aspects, the oxidase is glutamate oxidase. In still further aspects, the dehydrogenase is glucose dehydrogenase. In yet further aspects, the dehydrogenase is lactate dehydrogenase. In yet other aspects, the dehydrogenase is glutamate dehydrogenase.

The oxidase and/or dehydrogenase used in the oxidase or dehydrogenase domain can be derived from or encoded by a microorganism such as a bacteria, virus or fungus. Examples of sources for the oxidase or dehydrogenase domains used in the protein switches described herein are provided in Tables 1 and 2, below.

TABLE 1
Oxidases

| | | Reactant |
|---|---|---|
| Enzyme | Source | (Enzyme Substrate) |
| Glucose | Aspergillus niger EC 1.1.3.4, | B-D-Glucose |
| oxidase | Acremonium strictum, | |
| | Peniclium amagasakiense, or | |
| | flavin adenine dinucleotide | |
| | dependent glucose oxidase | |
| | from Burkholderia cepaciam | |
| Lactate | Pediococcus sp., Aerococcus | L-Lactate |
| oxidase | viridians EC 1.1.3.2 | |

TABLE 2
Dehydrogenases

| Enzyme | Source | Reactant |
|---|---|---|
| Glucose | Pseudomonas sp., | Glucose |
| dehydrogenase | Escherichia coli EC 1.1,1.47, | |
| | Burkholderia cepacian (such | |
| | as strain J2315), | |
| | Collectolrichum | |
| | gloeosporiodies, Aspergillus | |
| | niger, a nicotinamide adenine | |
| | dependent glucose | |
| | dehydrogenase from Bacillus | |
| | lichemformis or Bacillus | |
| | megaterium, an E.C 1.1.1.118 | |
| | NAD+ glucose | |
| | dehydrogenase from | |
| | Acientobacter calcoaceticus, | |
| | or a flavin adenine | |
| | dinucleotide dependent | |
| | glucose dehydrogenase from | |
| | Thermoascus aurantiacus, | |
| | Thermoascus crustaceus, | |
| | Talaromyces emersonii, | |
| | Aspergillus ustus or | |
| | Aspergillus bisporus | |
| Lactate | Rabbit muscle, chicken heart | L-Lactate |
| dehydrogenase | EC 1.1.1.27 | |

Methods of using the oxidases and dehydrogenases in Tables 1 and 2 in combination with one or more reactants (and one or more co-factors such as, for example, pyrroquinolinequinone (PQQ), flavin adenine dinucleotide (FAD), nicotinamide adenine dinucleotide (NAD), flavin mononucleotide (FMN)) to produce or generate detectable products (e.g., signals) are well known in the art. By way of example, methods for generating detectable products with glucose oxidase and glucose dehydrogenase will now be disclosed.

The enzyme glucose oxidase is comprised of two identical protein subunits and a co-factor at its active site (i.e., flavin adenine dinucleotide (FAD)). With FAD, glucose oxidase catalyzes the oxidation of its reactant glucose at its first hydroxyl group, utilizing molecular oxygen as the electron acceptor, to produce the products gluconolactone and hydrogen peroxide. The hydrogen peroxide product produced can be detected (such as, for example, by electrochemical oxidation at an electrode and the number of electron transfers detected) using routine techniques known in the art. The reaction can be summarized as shown below:

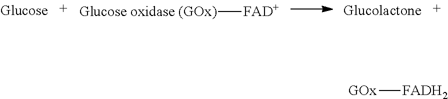

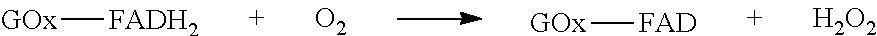

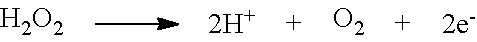

Alternatively, oxygen consumption can be measured.

Glucose dehydrogenase can utilize a number of different co-factors (e.g., NAD, PQQ, etc.). When NAD is used as a co-factor, glucose dehydrogenase catalyzes the oxidation of glucose to produce gluconolactone and NADH. The NADH can be electrochemically oxidized at an electrode and the number of electron transfers detected. The reaction can be summarized as shown below:

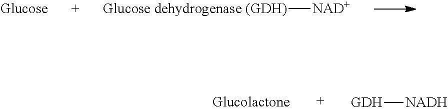

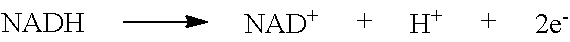

The target analytes detected by the protein switches can be prognostic or diagnostic for a patient's health and/or well-being. Alternatively, or in addition to, the target analytes can be used to monitor a condition of a patient's health and/or well-being. For example, the analyte can be procalcitonin (PCT), cardiac troponin (such as cardiac troponin I or cardiac troponin T), creatine, urea, guanidinosuccinic acid, para-cresol sulfate, indicant, dimethylamine, CMPF, pseudouridine, oxalate, glyoxal, 2-oxoglutarate, glucose, lactic acid, cerebral spinal fluid glucose, glutamic acid, malate, acetylcarnitine, hypoxanthine, glycerophosphocholine, acetylneuraminic acid, inosine, pseudouridine, hydroxyphenyllactic acid, hexanoylcarnitine, neuropeptide Y, orexin-A, calcitonin gene related peptide, serotonin, bran derived neurotrophic factor (BDNF), gamma-aminobutyric acid (GABA), dopamine, N-methyl-D-aspartate (NMDA), docosahexanoic acid (DHA), eicosapentaenoic acid (EPA), lysophosphatidylcholine (22:6 and 20:5), lysophosphatidyl-ethanolamine (22:6 and 20:5), 3-carboxy-40-methyl-5-propyl-2-furanopropanoic acid (CMPF), acetylcholine, cortisol, estrogen, estriol, estrone, progesterone, oxytocin, follicle stimulating hormone (FSH), luteinizing hormone (LH), thyroid stimulating hormone (TSH), triiodothyronine (T3), thyroxine (T4), triiodothryxin, human growth hormone, cytokines, chemokines, interleukins, procalcitonin, blood clotting factors, C reactive proteins (CRP), procalcitonin, soluble triggering receptor expressed on myeloid cells-1 (sTREM-1), pancreatic stone protein (PSP), circulating complement (C3 and C4), ferritin, cholesterol, albumin, and neutrophil gelatinase associated lipocalin. Analytes diagnostic for cardiac function or cardiac disease include, for example, cardiac troponin, 2-oxoglutarate, creatine kinase (CK-MB), glycogen phosphorylase isoenzyme BD, BNP, myoglobin, ischemia modified albumin, cardiac natriuretic peptides, and/or lactate dehydrogenase isozymes. The analyte may also be a nucleic acid (e.g., microRNAs, CpG islands, and other nucleic acid markers in the plasma or serum), polypeptides, metabolites, lipids, carbohydrates or other molecules found in a subject that can be diagnostic for the subject's health and/or well-being. Analytes diagnostic for kidney function or kidney disease include, for example, creatine and urea. Analytes diagnostic for liver function or liver disease include, for example, glucose, urea, albumin, and creatine. Analytes diagnostic for neural function or neural disease include GFAP, UCH-L1, S100B, and NF-L. Analytes diagnostic for infectious diseases and/or sepsis include for example, lactic acid, cerebral spinal fluid glucose, glutamic acid, malate, acetylcarnitine, hypoxanthine, glycerophosphocholine, acetylneuraminic acid, inosine, pseudouridine, hydroxyphenyllactic acid, hexanoylcarnitine, C reactive proteins (CRP), procalcitonin, soluble triggering receptor expressed on myeloid cells-1 (sTREM-1), pancreatic stone protein (PSP), circulating complement (C3 and C4), ferritin, cholesterol, albumin, cortisol, and neutrophil gelatinase associated lipocalin. Still other analytes include drugs or drug metabolites. For example, warfarin, methotrexate, cyclosporin A, methotrexate, and vasopressin. In one aspect, the analytes are warfarin, cortisol, methotrexate, triiodothyronine, cyclosporin A, GFAP, UCH-L1, S100B, NF-L or cardiac troponin. In yet another aspect, the analyte is warfarin. In still another aspect, the analyte is cortisol. In yet a further aspect, the analyte is methotrexate. In still a further aspect, the analyte is triiodothyronine. In still a further aspect, the analyte is cyclosporin A. In still a further aspect, the analyte is GFAP. In still a further aspect, the analyte is UCH-L1. In still a further aspect, the analyte is S100B. In yet still a further aspect, the analyte is NF-L. In still a further aspect, the analyte is cardiac troponin (e.g., troponin I or troponin T). In still a further aspect, the analyte is cardiac troponin I.

The analyte which binds to the analyte binding domain of the protein switch must be different (e.g., cannot be identical) to the reactant that binds and/or reacts with the oxidase or dehydrogenase domain. For example, if the analyte which binds to the analyte binding domain is glucose, then the reactant that binds or reacts with the oxidase or dehydrogenase domain cannot be glucose but must be a difference reactant such as lactate. Alternatively, if the analyte which binds to the analyte binding domain of the protein switch is warfarin, then the reactant that binds or reacts with the oxidase or dehydrogenase can be glucose or lactate.

As discussed previously herein, when the analyte binds to the analyte binding domain, the oxidase or dehydrogenase activity changes. In some aspects, when the analyte binds to the analyte binding domain, the oxidase or dehydrogenase activity increases (e.g., the amount of product produced increases). In other aspects, when the analyte binds to the analyte binding domain, the oxidase or dehydrogenase activity decreases (e.g., the amount of product produced decreases).

Any reactant known in the art for pairing with an enzyme are suitable for use with the protein switch described herein. Examples of reactants that can be used include glucose or lactate. In some aspects, the reactant is glucose. In other aspects, the reactant is lactate.

In another aspect, the protein switch of the present disclosure is a glucose dehydrogenase polypeptide. In yet another aspect, the protein switch is a glucose dehydrogenase polypeptide having at least 7 mutations in the amino acid sequence:

(SEQ ID NO: 20)
MYPDLKGKVVAITGAASGLGKAMAIRFGKEQAKVVINYYSNKQDPNEVKEE
VIKAGGEAVVVQGDVTKEEDVKNIVQTAIKEFGTLDIMINNAGLENPVPSH
EMPLKDWDKVIGTNLTGAFLGSREAIKYFVENDIKGNVINMSSVHEVIPWP
LFVHYAASKGGMKLMTETLALEYAPKGIRVNNIGPGAINTTINAGKFADPK
QKADVESMIPMGYIGEPEEIAAVAAWLASKEASYVTGITLFADGGMTQYPS
FQAGRG.

In one aspect, the protein switch comprises at least 7 mutations at amino acid positions 96, 155, 156, 159, 170, 198 and 252 of SEQ ID NO:20. Mutations that can be made at amino acid positions 96, 155, 156, 159, 170, 198 and 252 of SEQ ID NO:20 are shown in the below Table 3A.

TABLE 3A

| Amino | | |
|---|---|---|
| Acid | SEQ ID | |
| Position | NO: 20 | Mutation |
| 96 | E | C, F, M, W, Y |
| 155 | F | A, G, I, L, V |
| 156 | V | T, S |
| 159 | A | T, S |
| 170 | E | K, R, H |
| 198 | G | E, D |
| 252 | Q | A, G, I, L, V |

In another aspect, the protein switch comprises mutations at amino acid positions 96, 155, 156, 159, 170, 198 and 252 of SEQ ID NO:20 as shown above in Table 3A as well as at least one mutation at amino acid positions 11, 22, 45, 48, 55, 98, 137, 141, 149, 154, 166, 173, 184, 195, 219, 240 and/or 251 of SEQ ID NO:20 as shown below in Table 3B.

TABLE 3B

| Amino | | |
|---|---|---|
| Acid | SEQ ID | |
| Position | NO: 20 | Mutation |
| 11 | A | G, I, L, V |
| 22 | A | G, I, L, V |
| 45 | P | N, Q |
| 48 | V | A, G, I, L |
| 55 | A | D, E |
| 98 | P | A, G, I, L, V |
| 137 | K | F, W, Y |
| 141 | I | A, G, L, V |
| 149 | V | A, G, I, L |
| 154 | L | A, G, I, V |
| 166 | K | S, T |
| 173 | A | G, I, L, V |
| 184 | N | S, T |
| 195 | I | H, L, R |
| 219 | G | S, T |
| 240 | T | N, Q |
| 251 | T | A, G, I, L, V |

In yet another aspect, the protein switch comprises 7 mutations at amino acid position 96, 155, 156, 159, 170, 198 and 252 of SEQ ID NO:20.

In another aspect, the protein switch comprises 8 mutations at amino acid position 96, 98, 155, 156, 159, 170, 198 and 252 of SEQ ID NO:20. In another aspect, the protein switch comprises 8 mutations at amino acid position 96, 155, 156, 159, 170, 198, 219 and 252 of SEQ ID NO:20. In another aspect, the protein switch comprises 8 mutations at amino acid position 96, 155, 156, 159, 170, 195, 198 and 252 of SEQ ID NO:20. In another aspect, the protein switch comprises 8 mutations at amino acid position 96, 141, 155, 156, 159, 170, 198 and 252 of SEQ ID NO:20. In another aspect, the protein switch comprises 8 mutations at amino acid position 96, 155, 156, 159, 170, 173, 198 and 252 of SEQ ID NO:20. In another aspect, the protein switch comprises 8 mutations at amino acid position 55, 96, 155, 156, 159, 170, 198 and 252 of SEQ ID NO:20. In another aspect, the protein switch comprises 8 mutations at amino acid position 96, 137, 155, 156, 159, 170, 198 and 252 of SEQ ID NO:20. In another aspect, the protein switch comprises 8 mutations at amino acid position 48, 96, 155, 156, 159, 170, 198 and 252 of SEQ ID NO:20. In another aspect, the protein switch comprises 8 mutations at amino acid position 96, 155, 156, 159, 170, 184, 198 and 252 of SEQ ID NO:20. In another aspect, the protein switch comprises 8 mutations at amino acid position 96, 155, 156, 159, 166, 170, 198 and 252 of SEQ ID NO:20. In another aspect, the protein switch comprises 8 mutations at amino acid position 96, 155, 156, 159, 170, 198, 240 and 252 of SEQ ID NO:20.

In still yet another aspect, the protein switch comprises 9 mutations at amino acid position 45, 96, 149, 155, 156, 159, 170, 198 and 252 of SEQ ID NO:20. In still yet another aspect, the protein switch comprises 9 mutations at amino acid position 22, 96, 154, 155, 156, 159, 170, 198 and 252 of SEQ ID NO:20. In yet another aspect, the protein switch comprises 9 mutations at amino acid position 96, 141, 154, 155, 156, 159, 170, 198 and 252 of SEQ ID NO:20.

In still yet a further aspect, the protein switch comprises 10 mutations at amino acid position 12, 96, 155, 156, 159, 170, 195, 198, 251 and 252 of SEQ ID NO:20.

In yet another aspect, the protein switch comprises 7 mutations at amino acid position 96, 155, 156, 159, 170, 198 and 252 of SEQ ID NO:20. In these aspects, the protein switch comprises a cysteine or a phenylalanine at amino acid position 96, an alanine at amino acid position 155, a serine at amino acid position 156, a tyrosine at amino acid position 159, a lysine at amino acid position 170, a glutamic acid at amino acid position 198, and a lysine or valine at amino acid position 252 of SEQ ID NO:20.

In another aspect, the protein switch comprises 8 mutations at amino acid position 96, 98, 155, 156, 159, 170, 198 and 252 of SEQ ID NO:20. In these aspects, the protein switch comprises a cysteine or a phenylalanine at amino acid position 96, a leucine at amino acid position 98, an alanine at amino acid position 155, a serine at amino acid position 156, a tyrosine at amino acid position 159, a lysine at amino acid position 170, a glutamic acid at amino acid position 198, and a lysine or valine at amino acid position 252 of SEQ ID NO:20.

In another aspect, the protein switch comprises 8 mutations at amino acid position 96, 155, 156, 159, 170, 198, 219 and 252 of SEQ ID NO:20. In these aspects, the protein switch comprises a cysteine or a phenylalanine at amino acid position 96, an alanine at amino acid position 155, a serine at amino acid position 156, a tyrosine at amino acid position 159, a lysine at amino acid position 170, a glutamic acid at amino acid position 198, a threonine at amino acid position 219, and a lysine or valine at amino acid position 252 of SEQ ID NO:20.

In another aspect, the protein switch comprises 8 mutations at amino acid position 96, 155, 156, 159, 170, 195, 198 and 252 of SEQ ID NO:20. In these aspects, the protein switch comprises a cysteine or a phenylalanine at amino acid position 96, an alanine at amino acid position 155, a serine at amino acid position 156, a tyrosine at amino acid position 159, a lysine at amino acid position 170, an arginine at amino acid position 195, a glutamic acid at amino acid position 198, and a lysine or valine at amino acid position 252 of SEQ ID NO:20.

In another aspect, the protein switch comprises 8 mutations at amino acid position 96, 141, 155, 156, 159, 170, 198 and 252 of SEQ ID NO:20. In these aspects, the protein switch comprises a cysteine or a phenylalanine at amino acid position 96, a valine at amino acid position 141, an alanine at amino acid position 155, a serine at amino acid position 156, a tyrosine at amino acid position 159, a lysine at amino acid position 170, a glutamic acid at amino acid position 198, and a lysine or valine at amino acid position 252 of SEQ ID NO:20.

In another aspect, the protein switch comprises 8 mutations at amino acid position 96, 155, 156, 159, 170, 173, 198 and 252 of SEQ ID NO:20. In these aspects, the protein switch comprises a cysteine or a phenylalanine at amino acid position 96, an alanine at amino acid position 155, a serine at amino acid position 156, a tyrosine at amino acid position 159, a lysine at amino acid position 170, a glycine at amino acid position 173, a glutamic acid at amino acid position 198, and a lysine or valine at amino acid position 252 of SEQ ID NO:20.

In another aspect, the protein switch comprises 8 mutations at amino acid position 55, 96, 155, 156, 159, 170, 198 and 252 of SEQ ID NO:20. In these aspects, the protein switch comprises a glutamic acid at amino acid position 55, a cysteine or a phenylalanine at amino acid position 96, an alanine at amino acid position 155, a serine at amino acid position 156, a tyrosine at amino acid position 159, a lysine at amino acid position 170, a glutamic acid at amino acid position 198, and a lysine or valine at amino acid position 252 of SEQ ID NO:20.

In another aspect, the protein switch comprises 8 mutations at amino acid position 96, 137, 155, 156, 159, 170, 198 and 252 of SEQ ID NO:20. In these aspects, the protein switch comprises a cysteine or a phenylalanine at amino acid position 96, a tyrosine at amino acid position 137, an alanine at amino acid position 155, a serine at amino acid position 156, a tyrosine at amino acid position 159, a lysine at amino acid position 170, a glutamic acid at amino acid position 198, and a lysine or valine at amino acid position 252 of SEQ ID NO:20.

In another aspect, the protein switch comprises 8 mutations at amino acid position 48, 96, 155, 156, 159, 170, 198 and 252 of SEQ ID NO:20. In these aspects, the protein switch comprises an alanine at amino acid position 48, a cysteine or a phenylalanine at amino acid position 96, an alanine at amino acid position 155, a serine at amino acid position 156, a tyrosine at amino acid position 159, a lysine at amino acid position 170, a glutamic acid at amino acid position 198, and a lysine or valine at amino acid position 252 of SEQ ID NO:20.

In another aspect, the protein switch comprises 8 mutations at amino acid position 96, 155, 156, 159, 170, 184, 198 and 252 of SEQ ID NO:20. In these aspects, the protein switch comprises a cysteine or a phenylalanine at amino acid position 96, an alanine at amino acid position 155, a serine at amino acid position 156, a tyrosine at amino acid position 159, a lysine at amino acid position 170, a serine at amino acid position 184, a glutamic acid at amino acid position 198, and a lysine or valine at amino acid position 252 of SEQ ID NO:20.

In another aspect, the protein switch comprises 8 mutations at amino acid position 96, 155, 156, 159, 166, 170, 198 and 252 of SEQ ID NO:20. In these aspects, the protein switch comprises a cysteine or a phenylalanine at amino acid position 96, an alanine at amino acid position 155, a serine at amino acid position 156, a tyrosine at amino acid position 159, a serine at amino acid position 166, a lysine at amino acid position 170, a glutamic acid at amino acid position 198, and a lysine or valine at amino acid position 252 of SEQ ID NO:20.

In another aspect, the protein switch comprises 8 mutations at amino acid position 96, 155, 156, 159, 170, 198, 240 and 252 of SEQ ID NO:20. In these aspects, the protein switch comprises a cysteine or a phenylalanine at amino acid position 96, an alanine at amino acid position 155, a serine at amino acid position 156, a tyrosine at amino acid position 159, a lysine at amino acid position 170, a glutamic acid at amino acid position 198, a glutamine at amino acid position 240, and a lysine or valine at amino acid position 252 of SEQ ID NO:20.

In still yet another aspect, the protein switch comprises 9 mutations at amino acid position 45, 96, 149, 155, 156, 159, 170, 198 and 252 of SEQ ID NO:20. In these aspects, the protein switch comprises an asparagine at amino acid position 45, a cysteine or a phenylalanine at amino acid position 96, an alanine at amino acid position 149, an alanine at amino acid position 155, a serine at amino acid position 156, a tyrosine at amino acid position 159, a lysine at amino acid position 170, a glutamic acid at amino acid position 198, and a lysine or valine at amino acid position 252 of SEQ ID NO:20.

In still yet another aspect, the protein switch comprises 9 mutations at amino acid position 22, 96, 154, 155, 156, 159, 170, 198 and 252 of SEQ ID NO:20. In these aspects, the protein switch comprises a glycine at amino acid position 22, a cysteine or a phenylalanine at amino acid position 96, a glycine at amino acid position 154, an alanine at amino acid position 155, a serine at amino acid position 156, a tyrosine at amino acid position 159, a lysine at amino acid position 170, a glutamic acid at amino acid position 198, and a lysine or valine at amino acid position 252 of SEQ ID NO:20.

In yet another aspect, the protein switch comprises 9 mutations at amino acid position 96, 141, 154, 155, 156, 159, 170, 198 and 252 of SEQ ID NO:20. In these aspects, the protein switch comprises a cysteine or a phenylalanine at amino acid position 96, a valine at amino acid position 141, a glycine at amino acid position 154, an alanine at amino acid position 155, a serine at amino acid position 156, a tyrosine at amino acid position 159, a lysine at amino acid position 170, a glutamic acid at amino acid position 198, and a lysine or valine at amino acid position 252 of SEQ ID NO:20.

In still yet a further aspect, the protein switch comprises 10 mutations at amino acid position 12, 96, 155, 156, 159, 170, 195, 198, 251 and 252 of SEQ ID NO:20. In these aspects, the protein switch comprises a lysine at amino acid position 12, a cysteine or a phenylalanine at amino acid position 96, an alanine at amino acid position 155, a serine at amino acid position 156, a tyrosine at amino acid position 159, a lysine at amino acid position 170, an arginine at amino acid position 195, a glutamic acid at amino acid position 198, a lysine at amino acid position 251, and a lysine or valine at amino acid position 252 of SEQ ID NO:20.

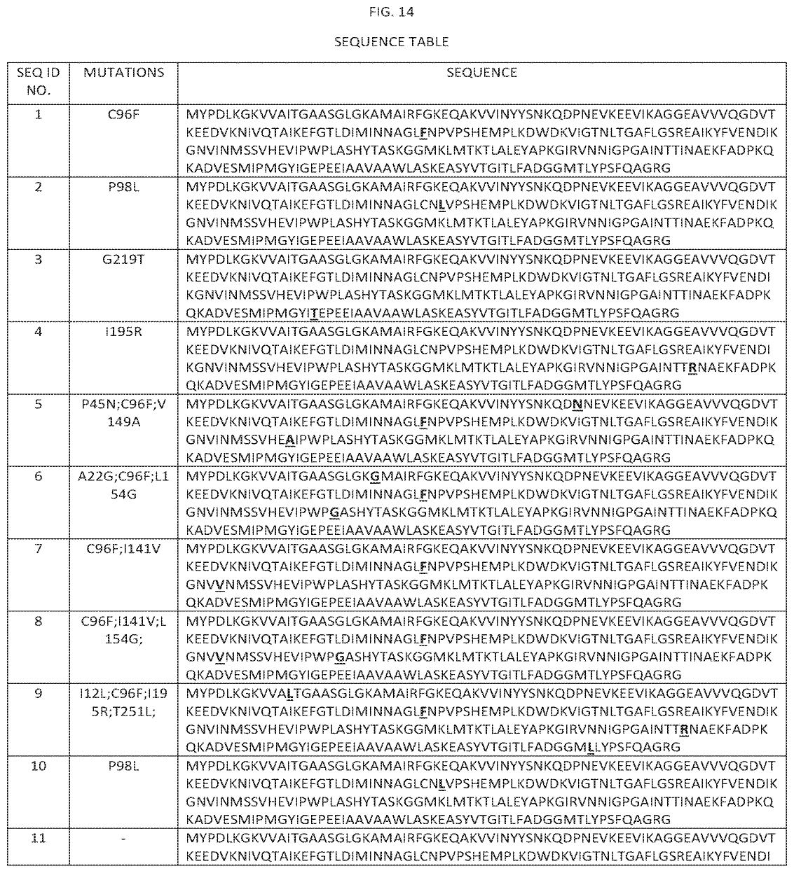

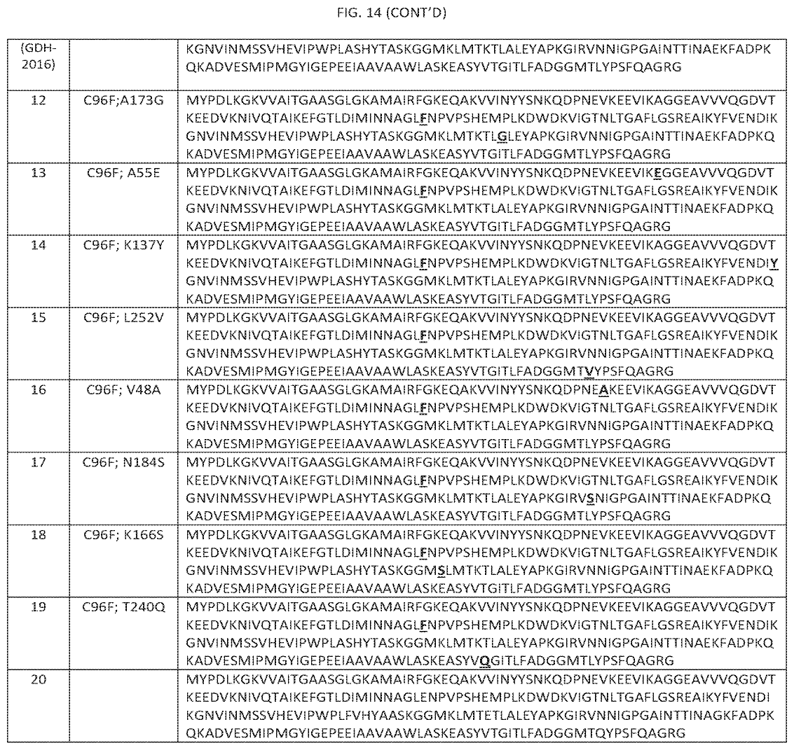

In yet another aspect, the protein switch of the present disclosure has the amino acid sequence of SEQ ID NO:11 with at least one of the following mutations (single) or combination of mutations listed in the below Table 3C and FIG. 14.

TABLE 3C

| Mutation | SEQ ID NO. |
|---|---|
| C96F | \grave{ } SEQ ID NO: 1 |
| P98L | SEQ ID NO: 2 |
| G219T | SEQ ID NO: 3 |
| I195R | SEQ ID NO: 4 |
| P45N; C96F; V149A | SEQ ID NO: 5 |
| A22G; C96F; L154G | SEQ ID NO: 6 |
| C96F; I141V | SEQ ID NO: 7 |
| C96F; I141V; L154G | SEQ ID NO: 8 |
| I12L; C96F; I195R; T251L | SEQ ID NO: 9 |
| P98L | SEQ ID NO: 10 |
| C96F; A173G | SEQ ID NO: 12 |
| C96F; A55E | SEQ ID NO: 13 |
| C96F; K137Y | SEQ ID NO: 14 |
| C96F; L252V | SEQ ID NO: 15 |
| C96F; V48A | SEQ ID NO: 16 |
| C96F; N184S | SEQ ID NO: 17 |
| C96F; K166S | SEQ ID NO: 18 |
| C96F; T240Q | SEQ ID NO: 19 |

In another aspect, the protein switch of the present disclosure has an amino acid sequence of any one of SEQ ID NOS. 1-19, an amino acid sequence having at least 60% sequence identity to any one of SEQ ID NOS. 1-19, at least 65% sequence identity to any of SEQ ID NOS. 1-19, at least 70% sequence identity to any one of SEQ ID NOS. 1-10 and 12-19, at least 75% sequence identity to any one of SEQ ID NOS. 1-19, at least 80% sequence identity to any one of SEQ ID NOS. 1-19, at least 85% sequence identity to any one of SEQ ID NOS. 1-19, at least 90% sequence identity to any one of SEQ ID NOS. 1-19, at least 95% sequence identity to any one of SEQ ID NOS. 1-19, at least 96% sequence identity to any one of SEQ ID NOS. 1-19, at least 97% sequence identity to any one of SEQ ID NOS. 1-19, at least 98% sequence identity to any one of SEQ ID NOS. 1-19, at least 99% sequence identity to any one of SEQ ID NOS. 1-19, or at least 100% sequence identity to any one of SEQ ID NOS. 1-19. The protein switch comprising an amino acid sequence of SEQ ID NOS. 1-19 binds the analyte warfarin.

In another aspect, the protein switch of the present disclosure has an amino acid sequence of any of SEQ ID NOS. 1, 7, 12 or 15, an amino acid sequence having at least 60% sequence identity to any one of SEQ ID NOS. 1, 7, 12 or 15, at least 65% sequence identity to any of SEQ ID NOS. 1, 7, 12 or 15, at least 70% sequence identity to any one of SEQ ID NOS. 1, 7, 12 or 15, at least 75% sequence identity to any one of SEQ ID NOS. 1, 7, 12 or 15, at least 80% sequence identity to any one of SEQ ID NOS. 1, 7, 12 or 15, at least 85% sequence identity to any one of SEQ ID NOS. 1, 7, 12 or 15, at least 90% sequence identity to any one of SEQ ID NOS. 1, 7, 12 or 15, at least 95% sequence identity to any one of SEQ ID NOS. 1, 7, 12 or 15, at least 96% sequence identity to any one of SEQ ID NOS. 1, 7, 12 or 15, at least 97% sequence identity to any one of SEQ ID NOS. 1, 7, 12 or 15, at least 98% sequence identity to any one of SEQ ID NOS. 1, 7, 12 or 15, at least 99% sequence identity to any one of SEQ ID NOS. 1, 7, 12 or 15, or at least 100% sequence identity to any one of SEQ ID NOS. SEQ ID NOS. 1, 7, 12 or 15.

In yet another aspect, the protein switches of the present disclosure include any of the enzyme of Examples 1 or 2.

As will be discussed in more detail herein, one or more protein switches of the present disclosure can be incorporated into one or more sensors using routine techniques known in the art for use in sensing systems to detect one or more analytes in a sample.

### C. Methods for Making Protein Switches

In some aspects, the present disclosure relates to methods of identifying, making and/or preparing protein switches. In certain aspects, the protein switches of the present disclosure can be made by screening, non-naturally occurring variant polypeptide expression libraries with one or more analytes of interest using biochemistry techniques known in the art. For example, variant polypeptide expression libraries encoding an oxidase (e.g., glucose oxidase or lactate oxidase) or a dehydrogenase (e.g., glucose dehydrogenase or lactate dehydrogenase) can be screened with one or more analytes of interest. In one aspect, the rate of formation of NADH, NADPH, FADH₂, or other reduced co-factor, can be monitored for an increase or decrease in absorbance or fluorescence and polypeptides exhibiting such an increase or decrease in enzyme activity selected. In some aspects, polypeptides exhibiting a decrease in enzyme activity as a result of screening with an analyte of interest are selected. In other aspects, polypeptides exhibiting an increase in enzyme activity as a result of screening with an analyte of interest are selected.

Once the oxidase or dehydrogenase variants exhibiting an increase or decrease in enzyme activity are identified, mutated and/or evolved protein switches (e.g., enzymes), having increased or decreased oxidase or dehydrogenase activity can be readily generated using routine techniques known in the art. See, for example, Ling, et al., Anal. Biochem., 254 (2): 157-78 (1997); Dale, et al., Methods Mol. Biol., 57:369-74 (1996); Smith, Ann. Rev. Genet., 19:423-462 (1985); Botstein, et al., Science, 229:1193-1201 (1985); Carter, “Site-directed mutagenesis,” Biochem. J., 237:1-7 (1986); Kramer, et al., Cell, 38:879-887 (1984); Wells, et al., Gene, 34:315-323 (1985); Current Opinion in Chemical Biology, 3:284-290 (1999); Christians, et al., Nature Biotechnology, 17:259-264 (1999); Crameri, et al., Nature, 391:288-291; Crameri, et al., Nature Biotechnology, 15:436-438 (1997); Zhang, et al., Proceedings of the National Academy of Sciences U.S.A., 94:45-4-4509; Crameri, et al., Nature Biotechnology 14:315-319 (1996); Stemmer, Nature, 370:389-391 (1994); Stemmer, Proceedings of the National Academy of Sciences, U.S.A., 91:10747-10751 (1994); WO 95/22625; WO 97/0078; WO 97/35966; WO 98/27230; WO 00/42651; WO 01/75767 and U.S. Pat. No. 6,537,746. To maximize any diversity, several of the above-described techniques can be used sequentially. Typically, a library of variant polynucleotides is created by one mutagenic or evolutionary technique and their expression products are screened to find the polypeptides having an increase or decrease in oxidase or dehydrogenase activity. Then, a second mutagenic or evolutionary technique is applied to polynucleotides encoding the most or least enzyme activity to create a second library, which in turn is screened for oxidase or dehydrogenase activity by the same technique. The process of mutating and screening can be repeated as many times as needed, including the insertion of point mutations, to arrive at a polynucleotide that encodes a protein switch with the desired activity, thermostability, co-factor preference, or other characteristics.

### D. Devices and Sensor Systems

In other aspects, the present disclosure relates to one or more sensors employing and/or containing one or more (e.g., multiple) protein switches described herein for detection and/or monitoring of at least one analyte. As will be discussed in more detail herein, such sensors can be prepared using routine techniques in the art. Such sensors can then be employed in one or more sensor systems. A general description of suitable sensor configurations and sensor systems employing these sensors utilizing the protein switches of the present disclosure are provided. However, this description should be understood as being non-limiting of the aspects disclosed herein and that alternative sensors and systems are contemplated as remaining within the scope of the present disclosure.

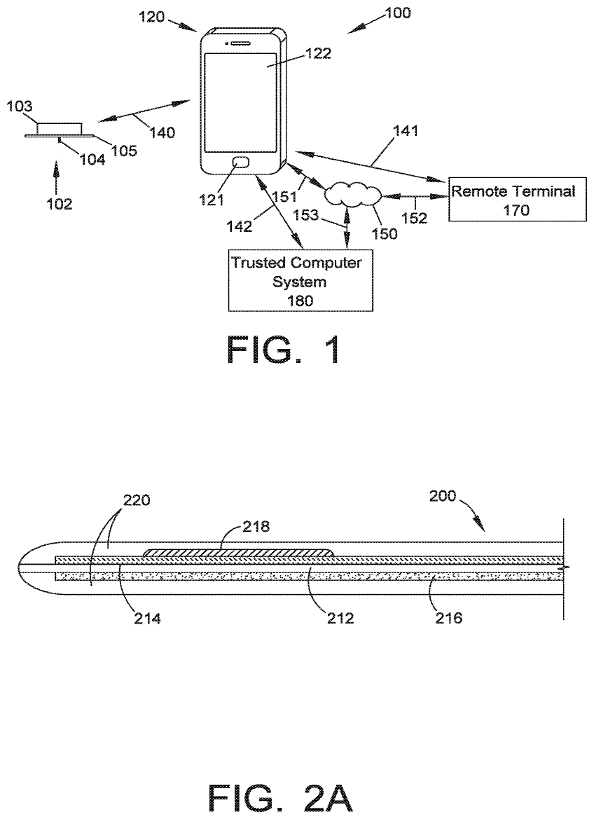

FIG. 1 provides a diagram of an illustrative sensing system incorporating a sensor comprising one or more protein switches of the present disclosure. As shown, sensing system 100 includes sensor control device 102 and reader device 120 (e.g., a signal detecting device) that are configured to communicate with one another over a local communication path or link, which may be wired or wireless, uni- or bi-directional, and encrypted or non-encrypted. Reader device 120 may constitute an output medium for viewing analyte concentrations and alerts or notifications determined by sensor/sensor 104 or a processor associated therewith, as well as allowing for one or more user inputs. Reader device 120 may be a multi-purpose smartphone or a dedicated electronic reader instrument. While only one reader device 120 is shown, multiple reader devices 120 may be present in certain instances. Reader device 120 may also be in communication with remote terminal 170 and/or trusted computer system 180 via communication path(s)/link(s) 141 and/or 142, respectively, which also may be wired or wireless, uni- or bi-directional, and encrypted or non-encrypted. Reader device 120 may also or alternately be in communication with network 150 (e.g., a mobile telephone network, the internet, or a cloud server) via communication path/link 151. Network 150 may be further communicatively coupled to remote terminal 170 via communication path/link 152 and/or trusted computer system 180 via communication path/link 153. Alternately, sensor 104 may communicate directly with other signal detecting devices such as a remote terminal 170 and/or trusted computer systems 180 without an intervening reader device 120 being present. For example, sensor 104 may communicate with remote terminal 170 and/or trusted computer system 180 through a direct communication link to network 150, according to some aspects, as described in U.S. Patent Application Publication 2011/0213225 an incorporated herein by reference in its entirety. Any suitable electronic communication protocol may be used for each of the communication paths or links, such as near field communication (NFC), radio frequency identification (RFID), BLUETOOTH® or BLUETOOTH® Low Energy protocols, Wi-Fi, or the like. Remote terminal 170 and/or trusted computer system 180 may be accessible, according to some aspects, by individuals other than a primary user who have an interest in the user's analyte levels. Reader device 120 may comprise display 122 and optional input component 121. Display 122 may comprise a touch-screen interface, according to some aspects.

Sensor control device 102 includes sensor housing 103, which may house circuitry and a power source for operating sensor 104. Optionally, the power source and/or active circuitry may be omitted. A processor (not shown) may be communicatively coupled to sensor 104, with the processor being physically located within sensor housing 103 or reader device 120. Sensor 104 protrudes from the underside of sensor housing 103 and extends through adhesive layer 105, which is adapted for adhering sensor housing 103 to a tissue surface, such as skin, according to some aspects.

Sensor 104 is adapted to be at least partially inserted into a tissue of interest, such as within the dermal or subcutaneous layer of the skin. Sensor 104 may comprise a sensor tail of sufficient length for insertion to a desired depth in a given tissue. The sensor tail may comprise at least one working electrode and one or more active areas (sensing layers or sensing regions/spots) located upon the at least one working electrode and that are active for sensing one or more analytes of interest. Collectively, the one or more active areas may comprise one or multiple protein switches. The active areas may include a polymeric material to which at least some of one or more of the protein switches are covalently bonded. In various aspects, analytes may be monitored in any biological fluid of interest such as dermal fluid, interstitial fluid, subcutaneous fluid, blood (e.g., such as intravenous or capillary). In particular aspects, sensors may be adapted for assaying dermal fluid or interstitial fluid.

In some aspects, sensor 104 may automatically forward data (e.g., such as by transmitting a signal) to reader device 120. For example, analyte concentration data (e.g., a signal representative of a level of one or more analytes) may be communicated automatically and periodically, such as at a certain frequency as data is obtained or after a certain time period has passed, with the data being stored in a memory until transmittal (e.g., every minute, five minutes, or other predetermined time period). In other aspects, sensor 104 may communicate with reader device 120 in a non-automatic manner and not according to a set schedule. For example, data may be communicated from sensor 104 using RFID technology when the sensor electronics are brought into communication range of reader device 120. Until communicated to reader device 120, data may remain stored in a memory of sensor 104. Thus, a patient does not have to maintain close proximity to reader device 120 at all times and can instead upload data at a convenient time. In yet other aspects, a combination of automatic and non-automatic data transfer may be implemented. For example, data transfer may continue on an automatic basis until reader device 120 is no longer in communication range of sensor 104.

An introducer may be present transiently to promote introduction of sensor 104 into a tissue. In some aspects, the introducer may comprise a needle or similar sharp. It is to be recognized that other types of introducers, such as sheaths or blades, may be present in alternative aspects. More specifically, the needle or other introducer may transiently reside in proximity to sensor 104 prior to tissue insertion and then be withdrawn afterward. While present, the needle or other introducer may facilitate insertion of sensor 104 into a tissue by opening an access pathway for sensor 104 to follow. For example, the needle may facilitate penetration of the epidermis as an access pathway to the dermis to allow implantation of sensor 104. After opening the access pathway, the needle or other introducer may be withdrawn so that it does not represent a sharps hazard. In other aspects, suitable needles may be solid or hollow, beveled or non-beveled, and/or circular or non-circular in cross-section. In more particular aspects, suitable needles may be comparable in cross-sectional diameter and/or tip design to an acupuncture needle, which may have a cross-sectional diameter of about 250 microns. It is to be recognized, however, that suitable needles may have a larger or smaller cross-sectional diameter if needed for particular applications.

In yet further aspects, a tip of the needle (while present) may be angled over the terminus of sensor 104, such that the needle penetrates a tissue first and opens an access pathway for sensor 104. In other illustrative aspects, sensor 104 may reside within a lumen or groove of the needle, with the needle similarly opening an access pathway for sensor 104. In either case, the needle is subsequently withdrawn after facilitating sensor insertion.

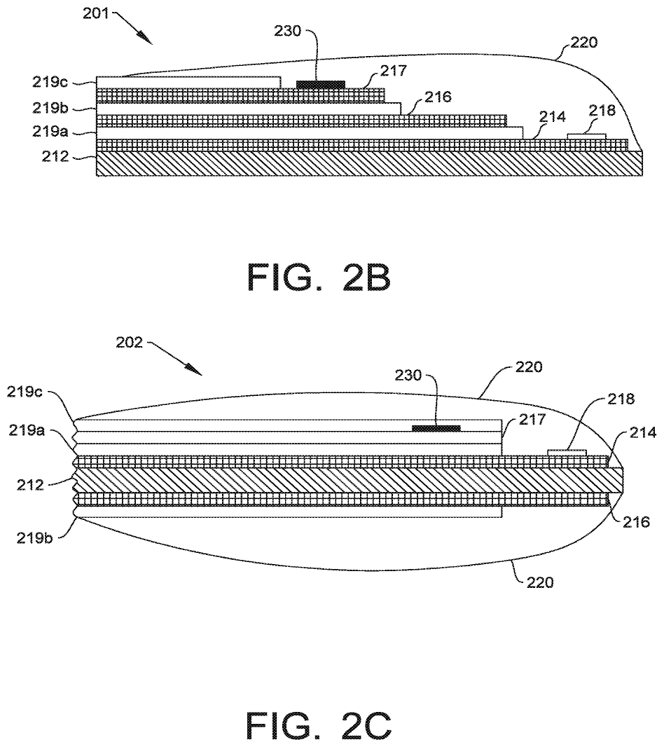

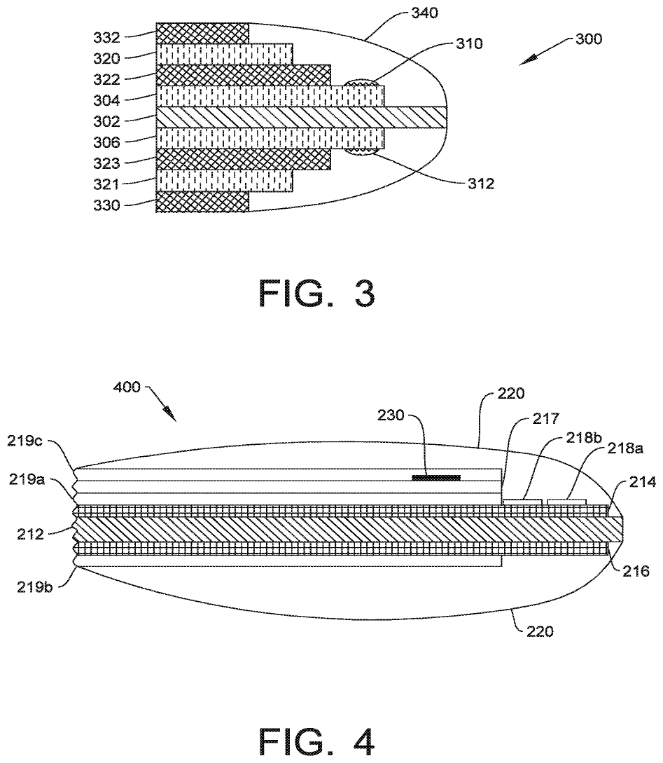

The sensors may contain or comprise one or more (e.g., multiple) protein switches upon the active area(s) of a single working electrode or upon two or more separate working electrodes. Single working electrode configurations for a sensor may employ two-electrode or three-electrode detection motifs. Sensor configurations featuring a single working electrode are described hereinafter in reference to FIGS. 2A-2C. Sensor configurations featuring multiple working electrodes are described separately thereafter in reference to FIG. 3. Multiple protein switches may be incorporated in any of the sensor configurations described hereinafter, with specific configurations suitable for incorporating the multiple protein switches being described in further detail hereinbelow.

When a single working electrode is present in a sensor, three-electrode detection motifs may comprise a working electrode, a counter electrode, and a reference electrode. Related two-electrode detection motifs may comprise a working electrode and a second electrode, in which the second electrode functions as both a counter electrode and a reference electrode (i.e., a counter/reference electrode). In both two-electrode and three-electrode detection motifs, one or more active areas of the sensor may be in contact with the working electrode. The one or more active areas may comprise multiple protein switches, with the one or more (e.g., multiple) protein switches being present in a single active area and/or in multiple active areas. In some aspects, the various electrodes may be at least partially stacked (layered) upon one another, as described in further detail hereinafter. In some or other aspects, the various electrodes may be laterally spaced apart from one another upon the sensor tail. Similarly, the associated active areas upon each electrode may be stacked vertically upon top of one another or be laterally spaced apart. In either case, the various electrodes may be electrically isolated from one another by a dielectric material or similar insulator.

FIG. 2A shows a diagram of an illustrative two-electrode sensor configuration having a single working electrode, which is compatible for use in some aspects of the disclosure herein. As shown, sensor 200 comprises substrate 212 disposed between working electrode 214 and counter/reference electrode 216. Alternately, working electrode 214 and counter/reference electrode 216 may be located upon the same side of substrate 212 with a dielectric material interposed in between (configuration not shown). Active area 218 is disposed as at least one layer upon at least a portion of working electrode 214. In various aspects, active area 218 may comprise multiple spots or a single spot configured for detection of one or more analytes of interest. One or more (e.g. multiple) protein switches may be present in active area 218 (i.e., in a single spot or in multiple spots).

Referring still to FIG. 2A, membrane 220 overcoats at least active area 218 and may optionally overcoat some or all of working electrode 214 and/or counter/reference electrode 216, or the entirety of sensor 200, according to some aspects. One or both faces of sensor 200 may be overcoated with membrane 220. Membrane 220 may comprise one or more polymeric membrane materials having capabilities of limiting analyte flux to active area 218. Depending on the identity of the analyte(s), the composition of membrane 220 may vary, as described further herein.

FIGS. 2B and 2C show diagrams of illustrative three-electrode sensor configurations having a single working electrode. Three-electrode sensor configurations employing a single working electrode may be similar to that shown for sensor 200 in FIG. 2A, except for the inclusion of additional electrode 217 in sensors 201 and 202 (FIGS. 2B and 2C). With additional electrode 217, counter/reference electrode 216 may then function as either a counter electrode or a reference electrode, and additional electrode 217 fulfills the other electrode function not otherwise accounted for. Working electrode 214 continues to fulfill its original function. Additional electrode 217 may be disposed upon either working electrode 214 or electrode 216, with a separating layer of dielectric material in between. For example, as depicted in FIG. 2B, dielectric layers 219a and 219b separate electrodes 214, 216 and 217 from one another and provide electrical isolation. Alternately, at least one of electrodes 214, 216 and 217 may be located upon opposite faces of substrate 212, as shown in FIG. 2C. Thus, in some aspects, electrode 214 (working electrode) and electrode 216 (counter electrode) may be located upon opposite faces of substrate 212, with electrode 217 (reference electrode) being located upon one of electrodes 214 or 216 and spaced apart therefrom with a dielectric material. Reference material layer 230 (e.g., Ag/AgCl) may be present upon electrode 217, with the location of reference material layer 230 not being limited to that depicted in FIGS. 2B and 2C. As with sensor 200 shown in FIG. 2A, active area 218 in sensors 201 and 202 may comprise multiple spots or a single spot configured for detection of one or more analytes of interest. One or more (e.g., multiple proteins switches) may be present in active area 218 of sensors 201 and 202.

Like sensor 200, membrane 220 may also overcoat active area 218, as well as other sensor components, in sensors 201 and 202. Additional electrode 217 may be overcoated with membrane 220 in some aspects. Although FIGS. 2B and 2C have depicted all of electrodes 214, 216 and 217 as being overcoated with membrane 220, it is to be recognized that only working electrode 214 may be overcoated in some aspects. Moreover, the thickness of membrane 220 at each of electrodes 214, 216 and 217 may be the same or different. As in two-electrode sensor configurations (FIG. 2A), one or both faces of sensors 201 and 202 may be overcoated with membrane 220 in the sensor configurations of FIGS. 2B and 2C, or the entirety of sensors 201 and 202 may be overcoated. Accordingly, the three-electrode sensor configurations shown in FIGS. 2B and 2C should be understood as being non-limiting of the aspects disclosed herein, with alternative electrode and/or layer configurations remaining within the scope of the present disclosure.

Sensor configurations having multiple working electrodes will now be described in further detail. Although the following description is primarily directed to sensor configurations having two working electrodes, it is to be appreciated that more than two working electrodes may be successfully incorporated through an extension of the disclosure herein. Additional working electrodes may allow additional active area(s) and corresponding sensing capabilities to be imparted to sensors having such features.

FIG. 3 shows a diagram of an illustrative sensor configuration having two working electrodes, a reference electrode and a counter electrode, which is compatible for use in some aspects of the disclosure. As shown in FIG. 3, sensor 300 includes working electrodes 304 and 306 disposed upon opposite faces of substrate 302. Active area 310 is disposed upon the surface of working electrode 304, and active area 312 is disposed upon the surface of working electrode 306. Collectively, one or more (e.g., multiple) protein switches may be present in active areas 310 and 312, with each active area 310, 312 containing one or more (e.g., multiple) protein switches. For example, a first protein switch comprising an analyte binding domain capable of binding to troponin and a glucose oxidase domain that is responsive to glucose may be present in active area 310 and a second protein switch comprising an analyte binding domain capable of binding to BNP and a glucose oxidase domain that is responsive to glucose may be present in active area 312 in particular aspects. Alternative, the second protein switch comprises an analyte binding domain capable of binding to BNP and a lactate oxidase domain that is responsive to lactate may be present in active area 312 in particular aspects. Counter electrode 320 is electrically isolated from working electrode 304 by dielectric layer 322, and reference electrode 321 is electrically isolated from working electrode 306 by dielectric layer 323. Outer dielectric layers 330 and 332 are positioned upon reference electrode 321 and counter electrode 320, respectively. Membrane 340 may overcoat at least active areas 310 and 312, according to various aspects. Other components of sensor 300 may be overcoated with membrane 340 as well, and as above, one or both faces of sensor 300, or a portion thereof, may be overcoated with membrane 340.

Alternative sensor configurations having multiple working electrodes and differing from that shown in FIG. 3 may feature a counter/reference electrode instead of separate counter and reference electrodes 320, 321, and/or feature layer and/or membrane arrangements varying from those expressly depicted. For example, the positioning of counter electrode 320 and reference electrode 321 may be reversed from that depicted in FIG. 3. In addition, working electrodes 304 and 306 need not necessarily reside upon opposing faces of substrate 302 in the manner shown in FIG. 3.

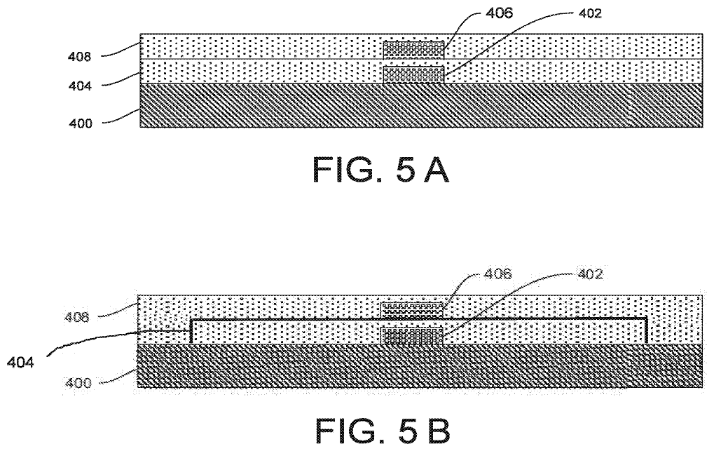

Sensor configurations featuring a working electrode having an active area remote therefrom are shown in FIGS. 5A and 5B and discussed in further below.

According some aspects, an electron transfer agent may be present in one or more of the sensing regions (e.g., active areas) of any of the sensors or sensor configurations disclosed herein. Suitable electron transfer agents/mediator compounds may facilitate conveyance of electrons to the working electrode when a reactant undergoes an oxidation-reduction reaction. Choice of the electron transfer agent within each active area may dictate the oxidation-reduction potential observed for each. When multiple active areas are present, the electron transfer agent within each active area may be the same or different.

Suitable electron transfer agents may include electroreducible and electrooxidizable ions, complexes or molecules (e.g., quinones) having oxidation-reduction potentials that are a few hundred millivolts above or below the oxidation-reduction potential of the standard calomel electrode (SCE). According to some aspects, suitable electron transfer agents may include low-potential osmium complexes, such as those described in U.S. Pat. Nos. 6,134,461 and 6,605,200, which are incorporated herein by reference in their entirety. Additional examples include those described in U.S. Pat. Nos. 6,736,957, 7,501,053 and 7,754,093, the disclosures of each of which are incorporated herein by reference in their entirety. Other suitable electron transfer agents may comprise metal compounds or complexes of ruthenium, osmium, iron (e.g., polyvinylferrocene or hexacyanoferrate), or cobalt, including metallocene compounds thereof, for example. Suitable examples of electron transfer mediators and polymer-bound electron transfer mediators may include those described in U.S. Pat. Nos. 8,444,834, 8,268,143 and 6,605,201, the disclosures of which are incorporated herein by reference in their entirety. Suitable ligands for the metal complexes may also include, for example, bidentate or higher denticity ligands such as, for example, bipyridine, biimidazole, phenanthroline, or pyridyl (imidazole). Other suitable bidentate ligands may include, for example, amino acids, oxalic acid, acetylacetone, diaminoalkanes, or o-diaminoarenes. Any combination of monodentate, bidentate, tridentate, tetradentate, or higher denticity ligands may be present in a metal complex to achieve a full coordination sphere.

In still other aspects, the active area or sensing region may also include a co-factor which is capable of catalyzing a reaction of the reactant associated with the at least one oxidase or dehydrogenase domain portion of the protein switch. In some aspects, the co-factor is a non-protein organic molecule such as, pyrroquinolinequinone (PQQ), flavine adenine dinucleotide (FAD), nicotinamide adenine dinucleotide (NAD), flavin mononucleotide (FMN), etc.). In certain aspects, a co-factor may be attached to a polymer, cross linking the co-factor with an electron transfer agent. A second co-factor may also be used in certain aspects.

In other aspects, a polymer may be present in each active area of any of the sensors or sensor configurations disclosed herein. Suitable polymers for inclusion in the active areas may include, but are not limited to, polyvinylpyridines (e.g., poly(4-vinylpyridine)), polyvinylimidazoles (e.g., poly(1-vinylimidazole)), or any copolymer thereof. Illustrative copolymers that may be suitable for inclusion in the active areas include those containing monomer units such as styrene, acrylamide, methacrylamide, or acrylonitrile, for example. When multiple active areas are present, the polymer within each active area may be the same or different.

In some aspects, the electron transfer agent may be covalently bonded to the polymer in each active area. The manner of covalent bonding is not considered to be particularly limited. Covalent bonding of the electron transfer agent to the polymer may take place by polymerizing a monomer unit bearing a covalently bound electron transfer agent, or the electron transfer agent may be reacted with the polymer separately after the polymer has already been synthesized. In other aspects, a bifunctional spacer may covalently bond the electron transfer agent to the polymer within the active area, with a first functional group being reactive with the polymer (e.g., a functional group capable of quaternizing a pyridine nitrogen atom or an imidazole nitrogen atom) and a second functional group being reactive with the electron transfer agent (e.g., a functional group that is reactive with a ligand coordinating a metal ion).

Similarly, in yet other aspects, the protein switch within one or more of the active areas may be covalently bonded to the polymer. When multiple proteins switches are present in a single active area, all of the multiple protein switches may be covalently bonded to the polymer in some aspects, and in other aspects, only a portion of the multiple protein switches may be covalently bonded to the polymer. For example, a first protein switch may be covalently bonded to the polymer and a second protein switch may be non-covalently associated with the polymer. According to more specific aspects, covalent bonding of the protein switch to the polymer may take place via a crosslinker introduced with a suitable crosslinking agent. Suitable crosslinking agents for reaction with free amino groups in the protein switch may include crosslinking agents such as, for example, polyethylene glycol diglycidylether (PEGDGE) or other polyepoxides, cyanuric chloride, N-hydroxysuccinimide, imidoesters, epichlorohydrin, or derivatized variants thereof. Suitable crosslinking agents for reaction with free carboxylic acid groups in the protein switch may include, for example, carbodiimides. The crosslinking is generally intermolecular but can be intramolecular in some aspects.

The electron transfer agent and/or the one or more (e.g., multiple) protein switches may be associated with the polymer in the active area through means other than covalent bonding as well. In some aspects, the electron transfer agent and/or the one or more (e.g., multiple) protein switches may be ionically or coordinatively associated with the polymer. For example, a charged polymer may be ionically associated with an oppositely charged electron transfer agent or protein switch. In still other aspects, the electron transfer agent and/or the protein switch may be physically entrained within the polymer without being bonded thereto.

As mentioned previously, various configurations suitable for arranging multiple protein switches in sensors are contemplated within the present disclosure. The multiple protein switches may be deposited within one or more active areas of the sensors. The active areas may range in size from about 0.01 mm² to about 1 mm², although larger or smaller active areas are also contemplated herein.

In other aspects, sensors containing multiple protein switches, whether operating independently or in concert, may function with enhanced stability in the presence of an appropriate stabilizer. Stabilizers that may be used include, for example, catalase or albumin (e.g., bovine serum albumin or human serum albumin).

In still yet other aspects, multiple protein switches may be arranged within separate active areas upon a single working electrode. When the multiple protein switches are arranged in this manner, each active area may facilitate detection of separate (e.g. two or more different) analytes, as described hereinafter. At least one of the active areas may produce a signal independently of the other active areas.

According to some aspects, sensors having multiple active areas upon a single working electrode may comprise: a sensor tail comprising at least a working electrode, and at least two active areas disposed upon a surface of the working electrode. Each active area comprises a protein switch and a polymer, with the protein switch in each active area being different. Each active area has an oxidation-reduction potential, and the oxidation-reduction potential of a first active area is sufficiently separated from the oxidation-reduction potential of a second active area to allow production of a signal from the first active area independent of a signal from the second active area. In more specific aspects, such sensors may comprise a single working electrode having the at least two active areas. An electron-transfer agent may be incorporated within each active area to promote electron transfer.

Alternative sensor configurations may comprise a single active area containing both the first protein switch and the second protein switch, along with an electron transfer agent. Each protein switch may be covalently bonded to separate portions of the polymer in the single active area. Provided that the sensing chemistries for promoting electron transfer for each analyte are not overly diluted in the single active area, the single active area may facilitate analyte detection in a manner similar to that described below for separate active areas. Such sensor configurations may be particularly feasible when the analytes to be assayed with the first and second protein switches have comparable membrane permeability values.

In more specific aspects, the sensor tail may be configured for insertion into a tissue. Suitable tissues are not considered to be particularly limited and are addressed in more detail above. Similarly, considerations for deploying a sensor tail at a particular position within a tissue are addressed above.

In other aspects, the oxidation-reduction potential associated with the first active area may be separated from the oxidation-reduction potential of the second active area by at least about 100 mV, or by at least about 150 mV, or by at least about 200 mV. The upper limit of the separation between the oxidation-reduction potentials is dictated by the working electrochemical window in vivo. By having the oxidation-reduction potentials of the active areas sufficiently separated in magnitude from one another, an electrochemical reaction may take place within the first active area without substantially inducing an electrochemical reaction within the second active area. Thus, a signal from the first active area may be independently produced at or above its corresponding oxidation-reduction potential. At or above the oxidation-reduction potential of the second active area, in contrast, electrochemical reactions may occur within both active areas. As such, the resulting signal at or above the oxidation-reduction potential of the second active area may include a signal contribution from both the first active area and the second active area, and the signal is a composite signal. The signal contribution from the second active area at or above its oxidation-reduction potential may then be determined by subtracting from the composite signal the signal obtained solely from the first active area at or above its corresponding oxidation-reduction potential. Similar considerations apply to analyzing the signal contributions from a single active area containing two different protein switches that produce signals at different oxidation-reduction potentials.

In more specific aspects, the first and second active areas may contain different electron transfer agents when the active areas are located upon the same working electrode, so as to afford oxidation-reduction potentials that are sufficiently separated in magnitude. More specifically, the first active area may comprise a first electron transfer agent and the second active area may comprise a second electron transfer agent, with the first and second electron transfer agents being different. The metal center and/or the ligands present in a given electron transfer agent may be varied to provide sufficient separation of the oxidation-reduction potentials of the first and second active areas, according to various aspects of the present disclosure. According to still more specific aspects, the first electron transfer agent may be covalently bonded to the polymer in the first active area, and the second electron transfer agent may be covalently bonded to the polymer in the second active area. The manner of covalent bonding for the first electron transfer agent and the second electron transfer agent may be the same or different. Similar considerations apply to choosing electron transfer agents suitable for use in conjunction with a first protein switch and a second protein switch contained within a single active area, in accordance with the disclosure above.

In more specific aspects of the present disclosure, the protein switch in each active area may be covalently bonded (or otherwise immobilized) to the polymer within each active area. In still more specific aspects, the protein switch and the electron transfer agent in each active area may be covalently bonded to the polymer within each active area. When contained in a single active area, the first protein switch and a first electron transfer agent may be covalently bonded to a first portion of polymer, and the second protein switch and a second electron transfer agent may be covalently bonded to a second portion of polymer. The polymer in the first portion and the second portion may be the same or different.

In some aspects, the first and second active areas located on a single working electrode may be configured to attain a steady state current rapidly upon operating the sensor at a given potential. Rapid attainment of a steady state current may be promoted by choosing an electron transfer agent for each active area that changes its oxidation state quickly upon being exposed to a potential at or above its oxidation-reduction potential. Making the active areas as thin as possible may also facilitate rapid attainment of a steady state current. For example, suitable thicknesses for the first and second active areas may range from about 0.1 microns to about 10 microns. In some or other aspects, combining a conductive material such as, for example, carbon nanotubes, graphene, or metal nanoparticles within one or more of the active areas may promote rapid attainment of a steady state current. Suitable amounts of conductive particles may range from about 0.1% to about 50% by weight of the active area, or from about 1% to about 50% by weight, or from about 0.1% to about 10% by weight, or from about 1% to about 10% by weight. Stabilizers may also be employed to promote response stability.

It is also to be appreciated that the sensitivity (output current) of the sensors toward each analyte may be varied by changing the coverage (area or size) of the active areas, the areal ratio of the active areas with respect to one another, the identity and thickness of the mass transport limiting membrane overcoating the active areas, and any combination thereof. Variation of these parameters may be conducted readily by one of ordinary skill in the art.

Although the foregoing description is primarily directed to sensors configured for detecting two different analytes, it is to be appreciated that the concepts above may be extended for detecting more than two analytes using a corresponding number of active areas located upon a single working electrode. Specifically, sensors employing more than two active areas and a corresponding number of different protein switches (and electron transfer agents) therein may be employed to detect a like number of different analytes in further aspects of the present disclosure. Provided that the oxidation-reduction potential of each active area is sufficiently separated from that of other active areas, the signal contribution from each active area may be analyzed in a manner related to that described above to provide the concentration of each analyte.

For example, the first active area may comprise a first protein switch comprising an analyte binding domain capable of binding to troponin and a glucose oxidase domain that is responsive to glucose and the second active area may comprise a second protein switch comprising an analyte binding domain capable of binding to BNP and a lactate oxidase domain that is responsive to lactate in addition to the suitable electron transfer agents and polymers discussed in more detail above. For example, sensors suitable for detecting troponin and BNP may comprise a working electrode having a first active area and a second active area disposed thereon, and a mass transport limiting membrane overcoating the first and second active areas upon the working electrode, in which the second active area comprises a polymer, and an analyte binding domain capable of binding to BNP and a lactate oxidase domain that is responsive to lactate covalently bonded to the polymer and the first active area comprises an analyte binding domain capable of binding to troponin and a glucose oxidase domain that is responsive to glucose covalently bonded to a polymer. Alternatively, the lactate oxidase domain can be substituted with glucose oxidase domain that is responsive to glucose. First and second electron transfer agents differing from one another may be present in each active area. In more specific aspects, the mass transport limiting membrane may comprise at least a crosslinked polyvinylpyridine homopolymer or copolymer. The composition of the mass transport limiting membrane may be the same or different where the mass transport limiting membrane overcoats each active area. In particular aspects, the mass transport limiting membrane overcoating the first active area may be single-component (contain a single membrane polymer) and the mass transport limiting membrane overcoating the second active area may be multi-component (contain two or more different membrane polymers, one of which is a polyvinylpyridine homopolymer or copolymer), either as a bilayer or homogeneous admixture.

Similarly, it is also to be appreciated that some sensors having two or more active areas located upon a given working electrode may comprise two or more protein switches in at least one of the active areas. According to more specific aspects, the two or more protein switches in a given active area may interact in concert to generate a signal proportional to the concentration of a single analyte. Thus, protein switches need not necessarily be present in a 1:1 ratio with a given selection of analytes. Sensors containing in concert interacting protein switches are described in further detail herein.

Accordingly, multi-analyte detection methods employing sensors featuring multiple protein switches arranged upon a single working electrode are also described herein. In various aspects, such methods may comprise: exposing a sensor to a fluid comprising at least one analyte. The sensor comprises a sensor tail comprising at least a working electrode, particularly a single working electrode, and at least two active areas disposed upon a surface of the working electrode. Each active area comprises a protein switch and a polymer, and the protein switch in each active area is different. Each active area has an oxidation-reduction potential, and the oxidation-reduction potential of a first active area is sufficiently separated from the oxidation-reduction potential of a second active area to allow production of a signal from the first active area independent of production of a signal from the second active area. The methods additionally comprise: obtaining a first signal at or above the oxidation-reduction potential of the first active area, such that the first signal is proportional to a concentration of a first analyte; obtaining a second signal at or above the oxidation-reduction potential of the second active area, such that the second signal is a composite signal comprising a signal contribution from the first active area and a signal contribution from the second active area; and subtracting the first signal from the second signal to obtain a difference signal, the difference signal being proportional to a concentration of the second analyte.

In more specific aspects, the oxidation-reduction potential associated with the first active area may be separated from the oxidation-reduction potential of the second active area by at least about 100 mV, or by at least about 150 mV, or by at least about 200 mV in order to provide sufficient separation for independent production of a signal from the first active area.

In some or other more specific aspects, the fluid is a biological fluid and the sensor is exposed to the biological fluid in vivo within an individual. Suitable biological fluids for analysis with sensors having at least two different active areas located upon a given working electrode may include any of the biological fluids discussed previously herein.

In some aspects, the signals associated with each active area may be correlated to a corresponding analyte concentration by consulting a lookup table or calibration curve for each analyte. A lookup table for each analyte may be populated by assaying multiple samples having known analyte concentrations and recording the sensor response at each concentration for each analyte. Similarly, a calibration curve for each analyte may be determined by plotting the sensor response for each analyte as a function of the concentration. According to some aspects, the calibration curve for sensors of the present disclosure may be linear.

A processor may determine which sensor response value in a lookup table is closest to that measured for a sample having an unknown analyte concentration and then report the analyte concentration accordingly. In some or other aspects, if the sensor response value for a sample having an unknown analyte concentration is between the recorded values in the lookup table, the processor may interpolate between two lookup table values to estimate the analyte concentration. Interpolation may assume a linear concentration variation between the two values reported in the lookup table. Interpolation may be employed when the sensor response differs a sufficient amount from a given value in the lookup table, such as variation of about 10% or greater.

Likewise, according to some or other various aspects, a processor may input the sensor response value for a sample having an unknown analyte concentration into a corresponding calibration curve. The sensor may then report the analyte concentration accordingly.

Aspects of sensors having two different active areas disposed upon a given working electrode may employ sensor configurations related to those depicted in FIGS. 2A-2C and described above. It is to be appreciated, however, that suitable sensors may also feature multiple working electrodes, such as the sensor configuration shown in FIG. 3, with at least one of the working electrodes having at least two active areas that differ from one another. It is also to be appreciated that other sensor configurations having two or more different active areas disposed upon the surface of a given working electrode also reside within the scope of the present disclosure. For example, the location, orientation or functionality of the working electrode and the counter and/or reference electrodes may differ from that shown in the figures herein.

FIG. 4 shows an illustrative sensor configuration compatible for use in some aspects of the disclosure herein, in which two different active areas are disposed upon the surface of a single working electrode. The sensor configuration of FIG. 4 bears most similarity to that of FIG. 2C. Where appropriate, common reference characters from FIG. 2C are used in FIG. 4 in the interest of clarity, and features having a common structure and/or function are not described again in further detail in the interest of brevity. Again, it is to be appreciated that other sensor configurations may similarly incorporate the features described below for FIG. 4.

Referring to FIG. 4, sensor 400 includes active areas 218a and 218b upon the surface of working electrode 214. Active area 218a includes a first electron transfer agent and a first protein switch that may be covalently bonded to a polymer comprising active area 218a. Active area 218b similarly includes a second electron transfer agent and a second protein switch that may be covalently bonded to a polymer comprising active area 218b. The first electron transfer agent and the second electron transfer agent may differ in composition so as to provide separation of the oxidation-reduction potentials of first active area 218a and second active area 218b. In particular aspects, active area 218b may comprise an analyte binding domain capable of binding to BNP and a lactate oxidase domain that is responsive to lactate, and active area 218a an analyte binding domain capable of binding to troponin and a glucose oxidase domain that is responsive to glucose. Alternatively, the lactate oxidase domain can be substituted with a glucose oxidase domain response to glucose.

The oxidation-reduction potentials of first active area 218a and second active area 218b may be sufficiently separated from one another to allow production of a signal from first active area 218a independent of signal production from second active area 218b. As such, sensor 400 may be operated at a first potential at which an oxidation-reduction reaction occurs within first active area 218a but not within second active area 218b. Thus, a first analyte (e.g., troponin) may be selectively detected at or above the oxidation-reduction potential of first active area 218a, provided that the applied potential is not high enough to promote an oxidation-reduction reaction with second active area 218b. A concentration of the first analyte may be determined from the sensor response of first active area 218a by referring to a lookup table or calibration curve.

At or above the oxidation-reduction potential of second active area 218b, separate oxidation-reduction reactions may take place simultaneously or near simultaneously within both first active area 218a and second active area 218b. As a result, the signal produced at or above the oxidation-reduction potential of second active area 218b may comprise a composite signal having signal contributions from both first active area 218a and second active area 218b. To determine the concentration of the second analyte (e.g., BNP) from the composite signal, the signal from first active area 218a at or above its corresponding oxidation-reduction potential may be subtracted from the composite signal to provide a difference signal associated with second active area 218b alone. Once the difference signal has been determined, the concentration of a second analyte may be determined by reference to a lookup table or calibration curve.

As mentioned previously, similar considerations also apply to separating a first signal and a second signal from a single active area containing two different protein switches in order to determine the concentrations of first and second analytes that differ from one another.

As discussed previously herein, the sensor configurations shown in FIGS. 2A-4 all feature one or more working electrodes having one or more active areas disposed directly upon a surface of each working electrode. In contrast, FIGS. 5A and 5B show diagrams of a working electrode in which a first active area is disposed directly upon a surface of the working electrode and a second active area is separated from (spaced apart from or remote from) the working electrode by a membrane. The working electrode configurations depicted in FIGS. 5A and 5B may be substituted for any of the particular working electrode configurations depicted in FIGS. 2A-4. That is, the working electrode configurations depicted in FIGS. 5A and 5B may be combined in any suitable way with a counter electrode and/or a reference electrode, membrane, substrates, and similar structures in a sensor.

As shown in FIG. 5A, working electrode 400 has active area 402 disposed directly upon a surface thereof. Active area 402 comprises a first protein switch covalently bound to a first polymer. Typically, an electron transfer agent is also present in active area 402, with the electron transfer agent also being covalently bound to the polymer. Active area 402 is overcoated with membrane 404. Membrane 404 may also overcoat the surface of working electrode 400, as depicted, as well as other portions of a sensor in which working electrode 400 is present. Membrane 404 isolates active area 406 from working electrode 400, such that electron exchange between the two is precluded. Active area 406 comprises a second protein switch covalently bound to a second polymer. Typically, an electron transfer agent is also present in active area 406, with the electron transfer agent also being covalently bound to the second polymer. Although FIG. 5A shows active area 406 disposed directly over active area 402, it is to be appreciated that they may be laterally spaced apart from one another in alternative configurations also compatible with the present disclosure. Membrane 408 overcoats active area 406, and optionally other sensor components, to provide mass transport limiting properties. Similarly, as shown in FIG. 5B, membrane 404 need not necessarily extend the same lateral distance as does membrane 408 upon working electrode 400. Indeed, membrane 404 in FIG. 5B overcoats active area 402 but only a portion of the surface of working electrode 400, with membrane 408 overcoating active area 406, the surface of membrane 404 and the remainder of the surface of working electrode 400 not overcoated by membrane 404. Active areas 402 and 406 may also laterally offset from one another in some aspects.

Membrane 408 is permeable to an analyte and any additional components needed to promote an enzymatic reaction in active area 406. Membrane 404, in contrast, is permeable to a product formed in active area 406. That is, an analyte reacts in active area 406 to form a first product, which then diffuses through membrane 404 and is subsequently reacted further in active area 402 to form a second product. The second product is subsequently detectable based on electron exchange with working electrode 400.

The first membrane polymer and the second membrane polymer may differ from one another, according to some aspects. The first membrane polymer may be crosslinked polyvinylpyridine, according to some aspects. Crosslinked polyvinylpyridine is readily permeable to acetaldehyde in the aspects of the present disclosure. The second membrane polymer may be a crosslinked polyvinylpyridine-co-styrene polymer, in which a portion of the pyridine nitrogen atoms were functionalized with a non-crosslinked poly(ethylene glycol) tail and a portion of the pyridine nitrogen atoms were functionalized with an alkylsulfonic acid group. Such second membrane polymers are readily permeable to both glucose and ethanol.

In still yet other aspects, multiple protein switches may be arranged within the active areas of separate working electrodes. As such, the signals associated with the enzymatic reaction occurring within each active area may be measured separately by interrogating each working electrode at the same time or at different times. The signal associated with each active area may then be correlated to the concentration of separate analytes.

As discussed previously herein, a membrane (i.e., a mass transport limiting membrane) may overcoat one or more of the active areas in a sensor in order to increase biocompatibility and to alter the analyte flux to the active areas. Such membranes may be present in any of the sensors disclosed herein. Because different analytes may exhibit varying permeability values within a given membrane, a sensor configured to analyze for multiple analytes may exhibit dissimilar sensitivities for each analyte. One approach for addressing differing sensitivity values may involve utilizing different membrane thicknesses over each active area. Although feasible, this approach may be difficult to put into practice from a manufacturing standpoint. Namely, it can be difficult to vary the membrane thickness at different locations using typical dip coating techniques that are used for membrane deposition. Another possible approach is to use active areas with different sizes for each analyte.

In some aspects, sensors featuring two or more protein switches arranged upon separate working electrodes may comprise: a sensor tail comprising at least a first working electrode and a second working electrode, a first active area located upon a surface of the first working electrode, a second active area located upon a surface of the second working electrode, a multi-component membrane overcoating the first active area, and a homogenous membrane overcoating the second active area. The first active area comprises a first polymer and a first protein switch that is reactive with a first analyte, and the second protein switch comprises a second polymer and a second protein switch that is reactive with a second analyte. The first protein switch and the second protein switch are different and are reactive with different analytes. The multi-component membrane comprises at least a first membrane polymer and a second membrane polymer that differ from one another. The homogeneous membrane comprises one of the first membrane polymer and the second membrane polymer.

Particular configurations of the multi-component membranes described above may comprise a bilayer membrane in some aspects or an admixture of the membrane polymers in other aspects. Surprisingly, bilayer membranes and admixed membranes may function to levelize the analyte permeability, as discussed further below.

Sensors of the present disclosure having two different active areas located upon separate working electrodes may employ a sensor configuration similar to that described above in FIG. 3 or a variant thereof. For example, in some aspects, a counter/reference electrode may replace separate counter and reference electrodes in a sensor bearing two or more working electrodes. Similarly, the layer configuration and arrangement within sensors having two different active areas located upon separate working electrodes may differ from that depicted in FIG. 3.

For example, in some aspects, sensors having multiple working electrodes may comprise active areas in which an electron transfer agent is covalently bonded to the polymer in each active area. In some or other aspects, such sensors may feature the first protein switch covalently bonded to the polymer in the first active area and the second protein switch covalently bonded to the polymer in the second active area. Again, in particular aspects, the first protein switch may be may comprise a first protein switch comprising an analyte binding domain capable of binding to troponin and a glucose oxidase domain that is responsive to glucose and the second protein switch may comprise a second protein switch comprising an analyte binding domain capable of binding to BNP and a lactate oxidase domain that is responsive to lactate. Alternatively, the lactate oxidase domain can be substituted with a glucose oxidase domain responsive to glucose.

In still yet further aspects, sensors having multiple working electrodes may comprise a sensor tail configured for insertion into a tissue.

In some aspects, a bilayer membrane may overcoat the first active area upon one of the working electrodes. The bilayer membrane comprises a first membrane polymer and a second membrane polymer that are layered upon one another over the active area. In more specific aspects, the first membrane polymer may be disposed directly upon the active area of a first working electrode, and the second membrane polymer may be disposed upon the first membrane polymer to define the bilayer membrane. In such aspects, the second membrane polymer is present in the homogenous membrane located upon the second working electrode. Such bilayer configurations may be prepared, in some aspects, by coating the first membrane polymer only upon the first working electrode (e.g., by spray coating, painting, inkjet printing, roller coating, or the like) and then coating the second membrane polymer upon both working electrodes at the same time (e.g., by dip coating or a similar technique). In other aspects, the bilayer membrane may be configured as above, with the first membrane polymer being located upon the second working electrode.

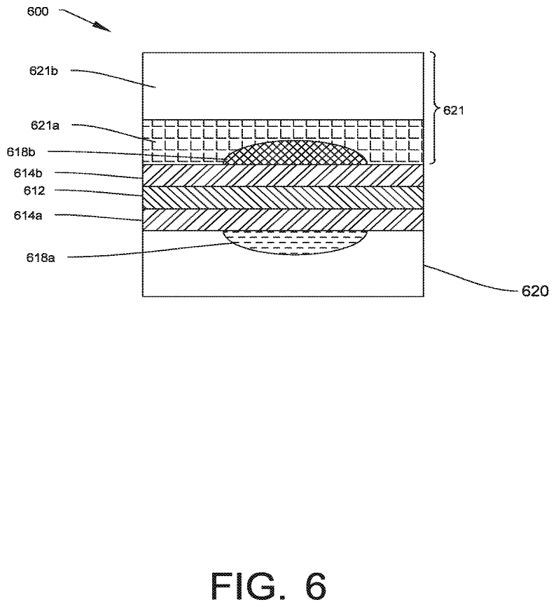

FIG. 6 shows an illustrative schematic of a portion of a sensor having two working electrodes and featuring a bilayer membrane overcoating one of the two working electrodes, which is compatible for use in some aspects of the disclosure herein. As shown in FIG. 6, the sensor features sensor tail 600 having working electrodes 614a and 614b disposed on opposite faces of substrate 612. Active area 618a is disposed upon working electrode 614a, and active area 618b is disposed upon working electrode 614b. Active areas 618a and 618b contain different protein switches and are configured to assay for different analytes, in accordance with the disclosure herein. Although FIG. 6 has shown active areas 618a and 618b to be disposed generally opposite on another with respect to substrate 612, it is to be appreciate that active areas 618a and 618b may be laterally spaced apart (offset) from one another upon opposite faces of substrate 612. Laterally spaced-apart configurations for active areas 618a and 618b may be particularly advantageous for overcoating each active areas 618a and 618b with mass transport limiting membranes, as discussed hereinafter.

As further shown in FIG. 6, active area 618a is overcoated with membrane layer 620. Membrane layer 620 is a homogenous membrane comprising a single membrane polymer. Active area 618b is overcoated with bilayer membrane 621, which comprises membrane layer 621a in direct contact with active area 618b and membrane layer 621b overlaying membrane layer 621a. Membrane layers 621a and 621b comprise different membrane polymers. As described above, in particular aspects, membrane layer 620 and membrane layer 621b may comprise the same membrane polymer.

Sensors having multiple active areas upon separate working electrodes, in which one of the active areas is overcoated with a bilayer membrane, may display levelized or independently variable analyte permeability, according to one or more aspects. That is, the sensors may have sensitivities for two different analytes that are closer to one another than if the bilayer membrane were not present. In such sensor configurations, the active area overcoated with the homogeneous membrane (e.g., membrane layer 620 in FIG. 6), may exhibit analyte permeability for a first analyte that is characteristic of its particular membrane polymer. Surprisingly, a bilayer membrane (e.g., bilayer membrane 621 in FIG. 6) may contain a membrane polymer that does not negatively impact the permeability of a second analyte (i.e., a membrane polymer having neutral permeability influence), thereby allowing the other membrane polymer comprising the bilayer membrane to exhibit its characteristic permeability for the second analyte as if the first membrane polymer was not present. Thus, according to various aspects, the membrane polymer having neutral permeability influence and the membrane polymer comprising the homogeneous membrane may constitute the same polymer.

In some or other specific aspects, the membrane polymer having neutral permeability influence may comprise the inner layer of the bilayer membrane. Thus, according to such aspects, the inner layer of the bilayer membrane and the homogeneous membrane may constitute the same membrane polymer. In other specific aspects, the outer layer of the bilayer membrane and the homogeneous membrane may constitute the same membrane polymer.

In still yet other aspects, the multi-component membrane may comprise an admixture (homogeneous blend) of the first membrane polymer and the second membrane polymer. Such sensor configurations may be similar in appearance to that shown in FIG. 6, except for replacement of bilayer membrane 621 with an admixed membrane comprising the two different membrane polymers in a homogeneous blend. As with sensors comprising a bilayer membrane disposed upon one of the active areas, a homogeneous membrane comprising one of the first membrane polymer or the second membrane polymer of the admixed membrane may overcoat the other active area upon the second working electrode.

Similar to a bilayer membrane, an admixed membrane containing a membrane polymer that neutrally influences the permeability of a second analyte may allow the admixed membrane to exhibit permeability for the second analyte that is largely characteristic of the other membrane polymer in the admixture. Thus, according to various aspects of the present disclosure, the membrane polymer of the homogeneous membrane and one of the membrane polymers of the admixed membrane may be chosen such that the permeability of the second analyte through the admixed membrane is not substantially altered by the membrane polymer. In particular aspects, the first active area may comprise a first protein switch comprising an analyte binding domain capable of binding to troponin and a glucose oxidase domain that is responsive to glucose, and the second active area may comprise a second protein switch comprising an analyte binding domain capable of binding to BNP and a lactate oxidase domain that is responsive to lactate. Thus, according to such aspects, the first active area containing the first protein switch may be overcoated with the admixed membrane, and the second active area containing the second protein switch may be overacted with the homogeneous (single-component membrane polymer) membrane. In yet other aspects, the second active area may comprise a polymer, an albumin, and a second protein switch covalently bonded to the polymer. In yet still more specific aspects, the homogenous membrane overcoating the second active area may comprise at least a crosslinked polyvinylpyridine homopolymer or copolymer, and the admixed membrane overcoating the first active area may also comprise the polyvinylpyridine homopolymer or copolymer.

As referenced above, bilayer membranes and admixed membranes may levelize analyte permeability in sensors described herein, wherein two or more active areas are spatially separated from one another and can be overcoated with different mass transport limiting membranes. Specifically, bilayer membranes and admixed membranes may levelize analyte permeability in sensors having separate working electrodes and comprising two or more active areas with different protein switches, with at least one active area being located at each working electrode. Thus, such membranes may advantageously allow the sensor sensitivity to be varied independently for each analyte. The membrane thickness and/or the relative proportion of the first membrane polymer to the second membrane polymer represent other parameters that may be varied to adjust the characteristic permeability of the analytes at each working electrode.

Accordingly, methods for using a sensor containing two working electrodes may comprise exposing a sensor to a fluid comprising at least one analyte. The sensor comprises a sensor tail comprising at least a first working electrode and a second working electrode. A first active area is disposed upon a surface of the first working electrode, and a second active area is disposed upon a surface of the second working electrode. The first active area comprises a first polymer and a first protein switch reactive with a first analyte, and the second active area comprises a second polymer and a second protein switch reactive with a second analyte. The first protein switch and the second protein switch are different. A multi-component membrane overcoats the first active area, and a homogeneous membrane overcoats the second active area. The multi-component membrane comprises at least a first membrane polymer and a second membrane polymer that differ from one another, and the homogeneous membrane comprises one of the first membrane polymer or the second membrane polymer. The methods further include obtaining a first signal at or above an oxidation-reduction potential of the first active area, obtaining a second signal at or above the oxidation-reduction potential of the second active area, and correlating the first signal to the concentration of the first analyte in the fluid and the second signal to the concentration of the second analyte in the fluid. The first signal is proportional to the concentration of the first analyte in the fluid, and the second signal is proportional to the concentration of the second analyte in the fluid.

In other aspects, aspects, the first signal and the second signal maybe measured at different times. Thus, in such aspects, a potential may be alternately applied to the first working electrode and the second working electrode. In other aspects, the first signal and the second signal may be measured simultaneously via a first channel and a second channel, in which case a potential may be applied to both electrodes at the same time.

In some aspects, the sensor system can also contain a reactant storage device that can act as a reservoir holding reactant for the enzyme portion of the protein switch, wherein the reactant is not available from the analyte-containing sample. Examples of reactant are glucose, lactate, etc. The reactant storage device can release reactant over an extended period of time. The storage device can be capable of delivering reactant to the protein switch for at least 1, 2, 3, 4, 5, 10, 15, 20, 30, 40, or 50 minutes, or 1, 2, 3, 4, 5, 6, 7, 8, 9, 10, 11, or 12 hours, or 1, 2, 3, 4, 5, 6, or 7 days, or 2, 3, or 4 weeks, or 2, 3, 4, 5, or 6 months. The enzyme storage device can include a mechanical device or an osmotic device for extended delivery of reactant from the enzyme storage device. The enzyme storage device can include a mechanical device or an osmotic device for extended delivery of reactant from the enzyme storage device. The enzyme storage device can also have combinations of the above for providing extended release of reactant. Other methods and technologies can also be used for extended release of the reactant.

The storage device can have a pore or hole through which the reactant is released. The pore or hole through which reactant can be released to the protein switch can be small and delivery of reactant from the storage device can occur over an extended period of time. Such holes or pores can be made by etching, laser machining, mechanical machining, drilling, and/or conventional processes.

A membrane or coating can be placed over the pore or hole of a storage device to extend the delivery time of reactant from the storage device. The storage device can be constructed of a porous material or a portion of the storage device can be constructed of a porous material, and the porous material can be coated with a polymeric coating and/or a membrane that retards release of the reactant from the storage device extending the release time of the reactant. The coatings can be made from polymer dispersions such as polyurethane dispersions (BAYHDROL™, etc.), acrylic latex dispersions, and EUDRAGIT®.

The reactant storage device can provide delivery of reactant by an osmotic delivery system. A “standard osmotic delivery system” that can be used is an elementary osmotic pump (EOP) system. EOPs are well known. This system can be used to deliver reactants and is especially suited for reactants which give “erratic” release profiles or give an incomplete release profile. See, e.g., Felix Theeuwes, Elementary Osmotic Pump, Journal of Pharmaceutical Sciences, Vol. 64, No. 12, pp 1987-1991 December 1975. The reactant storage device can include water-soluble compounds suitable for inducing osmosis, i.e. osmotic agents or osmogents, including pharmaceutically acceptable and pharmacologically inert water-soluble compounds referred to in the pharmacopias such as United States Pharmacopia, as well as in Remington: The Science and Practice of Pharmacy; edition 19; Mack Publishing Company, Easton, Pa. (1995). The osmotic agent can be selected from pharmaceutically acceptable water-soluble salts of inorganic or organic acids, or non-ionic organic compounds with high water solubility, e.g., carbohydrates such as sugar, or amino acids. The osmotic agent can also include inorganic salts such as magnesium chloride or magnesium sulfate, lithium, sodium or potassium chloride, lithium, sodium or potassium hydrogen phosphate, lithium, sodium or potassium dihydrogen phosphate, salts of organic acids such as sodium or potassium acetate, magnesium succinate, sodium benzoate, sodium citrate or sodium ascorbate; carbohydrates such as mannitol, sorbitol, arabinose, ribose, xylose, glucose, fructose, mannose, galactose, sucrose, maltose, lactose, raffinose; water-soluble amino acids such as glycine, leucine, alanine, or methionine; urea and the like, and mixtures thereof. The amount of osmogents that can be used depends on the particular osmogent that is used and can range from about 1% to about 60% by weight of the reactant mixture. The osmotic delivery system can also include a polymer, such as those described above as drug eluting polymers. The osmotic delivery system can also include a coating and/or membrane acting as a semipermeable barrier between the enzyme and the reactant. Other suitable membranes and/or coatings are known and can be used.

The reactant storage device can include or be part of a microfluidics systems for delivering reactant to the protein switch. Microfluidic systems for delivering reactant to the enzymes on a time extended basis are well known in the art to persons of ordinary skill. See, e.g., the microfluidics disclosed in U.S. Pat. Nos. 9,194,859 and 8,460,607, Madou, Fundamentals of Microfabrication: The Science of Miniaturization, Second Edition (Hardcover), published by CRC, 2002; Nguyen, et al., Fundamentals and Applications of Microfluidics, Artech House Publishers, (2002).

### E. Methods of Using Protein Switch Containing Devices and Sensor Systems

The devices containing one or more protein switches can detect, identify and/or monitor any of the analytes described in Section B, infra. For example, the protein switch containing devices can be used to monitor patients who are at risk for adverse cardiac events. For example, the device can detect cardiac troponin and the device can be used to monitor patients at risk for myocardial infarctions. The device can also be used to monitor cardiac patients after surgery or other treatment to monitor cardiac function and provide early warnings of potential adverse cardiac events.

In some aspects, the protein switch containing device can be used to monitor a subject's exposure to an infectious agent. For example, the device can be used to monitor a patient after an acute exposure to an infectious agent and/or to follow the progress of the infection. In other aspects, the device can be used to monitor a subject's response to anti-infectives and/or procedures administered to the subject to treat the infectious disease. In yet further aspects, the device can also be used to personalize treatment so that the subject receives adequate treatment to treat their sepsis or infection.

In still yet further aspects, the device can also be used to monitor drug treatment in a subject being treated with one or more drugs. In yet other aspects, the protein switch containing device can be used to detect the presence of one or more drugs and/or drug metabolites.

The protein switch containing device can be used to detect analyte(s) automatically. The device can detect changes in the analyte amounts or levels in a bodily fluid. The device can detect a rate of change of an analyte(s) in a bodily fluid. The device can detect when an analyte(s) reaches a threshold level or amount. Additionally, the device can monitor multiple analytes automatically in a bodily fluid of a subject.

The disclosure herein will be better understood from the experimental details which follow. However, one skilled in the art will readily appreciate that the specific methods and results discussed are merely illustrative as described more fully in the claims which follow thereafter. Unless otherwise indicated, the disclosure is not limited to specific procedures, materials, or the like, as such can vary. It is also to be understood that the terminology used herein is for the purpose of describing particular aspects only and is not intended to be limiting.

### EXAMPLES

### Example 1—Creation and Identification of Protein Switches

Glucose dehydrogenase (GDH) variant libraries comprising GDH polypeptides developed using GDH-105 (Codexis, Redwood, CA) as a backbone were prepared. Approximately 2800 GDH variants were prepared. The variants were screened for glucose activity against one or more of the analytes listed in Table 4 below. Glucose activity was determined by either NADH kinetic assay using a spectrophotometer set at UV 340 or UV 360 at various time intervals or by endpoint measurement of gluconic acid using RapidFire mass spectrometer.

Table 4: Reaction mixtures and analyte amounts

TABLE 4

| | Amount | | | | | | |
|---|---|---|---|---|---|---|---|
| | of | | | | | | |
| | analyte | | | GDH | 200 | | Glucose |
| | tested | NAD+ | Reactant | Lysate | mM | Analyte | activity |
| Analyte | (mM) | (g/L) | (g/L) | (v/v) | TEoA | Solvent | determination |
| Cortisol | 2.08 | 2 | 0.375 | 0.3% | Yes, | 10% v/v | NADH |
| | | | Glucose | | pH 7.5 | MeOH | kinetics at |
| | | | | | | | UV340 |
| Methotrexate | 5.42 | 2 | 0.375 | 0.3% | Yes, | 10% v/v | Gluconic acid |
| | | | Glucose | | pH 7.0 | DMSO | by RapidFire |
| | | | | | | | MS |
| T3 | 0.46 | 2 | 0.375 | 0.3% | Yes, | 10% v/v | NADH |
| | | | Glucose | | pH 7.5 | DMSO | kinetics at |
| | | | | | | | UV340 |
| Warfarin | 2.08 | 2 | 0.375 | 0.3% | Yes, | 10% v/v | NADH |
| | | | Glucose | | pH 7.0 | DMSO | kinetics at |
| | | | | | | | UV360 |

Table 5 shows the protein switches identified as binding to one or more of the analytes tested and whether the binding of the analyte to the protein switch inhibited or activated glucose activity.

TABLE 5

| Protein Switch | Analyte | Activate/Inhibit |
|---|---|---|
| GDH-2009 | Warfarin | Inhibit |
| GDH-2016 | Warfarin | Inhibit |
| GDH-2004 | Warfarin | Inhibit |
| GDH-2013 | Warfarin | Inhibit |
| GDH-2004 | Cortisol | Inhibit |
| GDH-2005 | Cortisol | Inhibit |
| GDH-2007 | Methotrexate | Inhibit |
| GDH-2008 | Methotrexate | Inhibit |
| GDH-2009 | Methotrexate | Inhibit |
| GDH-2010 | Methotrexate | Inhibit |
| GDH-2004 | Methotrexate | Inhibit |
| GDH-2011 | Methotrexate | Inhibit |
| GDH-2015 | Methotrexate | Inhibit |
| GDH-2018 | T3 | Inhibit |
| GDH-2019 | T3 | Inhibit |

### Example 2—Evolution of Protein Switches for Warfarin

Protein switch GDH-2016 (also known as Rd2bb) having amino acid sequence of SEQ ID NO:11, was evolved using routine techniques known in the art to create a library of variants. Approximately 3024 variants were prepared.

Each of the variants were screened for glucose activity and warfarin inhibition (% inhibition) by combining each variant with a reaction mixture containing 1.25 g/L glucose, 5 g/L NAD+, 20% HTP GDH-NAD lysate, with or without 0.1 g/L warfarin in 50 mM NaPO₄ at pH 7.0. Glucose activity was evaluated by NADH kinetic assay using a spectrophotometer (Molecular Devices SpectraMax M5) set at UV 360 and read for 8 minutes at 1-minute intervals. Top variants that showed improved inhibition (vs Rd2bb) were grown in shake flask scale and further tested for warfarin inhibition (Ki) by combining each variant with a reaction mixture containing 0-10 g/L glucose, 25 g/L NAD+, 0-0.5 g/L warfarin, GDH-NAD shake flask powder loaded in the range of 0.5 to 2.5 g/L depending on individual variant, in 50 mM NaPO₄ at pH 7.0. Protein switches exhibiting low Ki (high glucose inhibition) and high glucose activity (e.g., catalytic efficiency) as shown below in Table 6 were identified. These protein switches were determined to have the amino acid sequences shown in SEQ ID NOS.: 1-10. A protein switch having the sequence of SEQ ID NO.1 (GDH-2025; also known as Rd3bb) was selected for further evolution.

TABLE 6

| | Catalytic | Warfarin | | SEQ |
|---|---|---|---|---|
| Protein Switch | efficiency | Inhibition | Active | ID |
| Identifier | (Vmax/Km) | (Ki) | Mutations | NO. |
| AB_WF_2.1.-3 | 159.40 | 0.004657 | C96F | 1 |
| (GDH-2025) | | | | |
| AB_WF_2.1-4 | 89.93 | 0.009731 | P98L | 2 |
| AB_WF_2.1-5 | 128.82 | 0.007808 | G219T | 3 |
| (GDH-2024) | | | | |
| AB_WF_2.1-6 | 108.55 | 0.01003 | I195R | 4 |
| AB_WF_2.2-5-1 | 95.58 | 0.00624 | P45N; C96F; | 5 |
| | | | V149A | |
| AB_WF_2.2-5-2 | 113.84 | 0.0125 | A22G; C96F; | 6 |
| | | | L154G | |
| AB_WF_2.2-5-4 | 235.21 | 0.009296 | C96F; I141V | 7 |
| (GDH-2026) | | | | |
| AB_WF_2.2-5-5 | 114.62 | 0.00704 | C96F; I141V; | 8 |
| | | | L154G | |
| AB_WF_2.2-5-6 | 162.02 | 0.004475 | I12L; C96F; | 9 |
| | | | I195R; T251L | |
| AB_WF_2.2-5-7 | 187.50 | 0.007445 | P98L | 10 |

Protein switch GDH-2025 was evolved using similar techniques described above. Approximately 1680 variants were prepared. Each of the variants were screened for glucose activity and inhibition (Ki) by combining each variant with a reaction mixture containing 3.5 g/L glucose, 12.5 NAD+, 10% HTP GDH-NAD lysate, 0.003 g/L sodium warfarin in 50 mM NaPO₄ at pH 7.0. Glucose activity was evaluated using a spectrophotometer (Molecular Devices SpectraMax M5) set at UV 360 and read for 8 minutes at 1-minute intervals. Top variants that showed improved inhibition (vs Rd3bb) were grown in shake flask scale and further tested for warfarin inhibition (Ki) by combining each variant with a reaction mixture containing 0-10 g/L glucose, 25 g/L NAD+, 0-0.5 g/L warfarin, 0.5 g/L GDH-NAD shake flask powder in 50 mM NaPO₄ at pH 7.0. Protein switches exhibiting low Ki (high glucose inhibition) and high glucose activity (e.g., catalytic efficiency) as shown below in Table 7 were identified. These protein switches were determined to have the amino acid sequences shown in SEQ ID NOS.: 12-19. A protein switch having the sequence of SEQ ID NO.15 (GDH-2028; also known as Rd4bb) was selected for further evolution.

TABLE 7

| | Catalytic | Warfarin | | SEQ |
|---|---|---|---|---|
| Protein Switch | efficiency | Inhibition | Active | ID |
| Identifier | (Vmax/Km) | (Ki) | Mutations | NO. |
| AB_WF_3.1-10 | 109.81 | 0.004258 | C96F; A173G | 12 |
| (GDH-2027) | | | | |
| AB_WF_3.1-4 | 45.08 | 0.006119 | C96F; A55E | 13 |
| AB_WF_3.1-8 | 84.38 | 0.00529 | C96F; K137Y | 14 |
| AB_WF_3.1-2 | 61.66 | 0.002081 | C96F; L252V | 15 |
| (GDH-2028) | | | | |
| AB_WF_3.1-3 | 135.22 | 0.005161 | C96F; V48A | 16 |
| AB_WF_3.1-5 | 77.45 | 0.004161 | C96F; N184S | 17 |
| AB_WF_3.1-6 | 140.72 | 0.005488 | C96F; K166S | 18 |
| AB_WF_3.1-9 | 93.5 | 0.004018 | C96F; T240Q | 19 |

### Example 3—Protein Switch Sensors

Sensor Preparation. Experimental GDH-105 (control), GDH-2004 (methotrexate (MTX)), GDH-2007 (MTX), GDH-2008 (MTX), GDH-2009 (MTX), GDH-2010 (MTX), GDH-2011 (MTX), GDH-2015 (MTX), GDH-2018 (T3), GDH-2019 (T3), GDH-2004 (cortisol (CT)), GDH-2005 (CT), GDH-2004 (warfarin (WF)), GDH-2009 (WF), GDH-2013 (WF), GDH-2016 (WF), and GDH-2024 (WF) glucose-responsive active areas were prepared using a single-layer active area system. The GDH was obtained from Codexis, Redwood City, CA. The active area was coated onto a carbon working electrode in a single layer composition (Table 8 below). Following deposition of the active area (0.11 mm²), the active area was cured for three days at room temperature. Thereafter, a PVP membrane was applied to the working electrode using a membrane coating solution comprising 4 mL of 100 mg/mL polyvinyl pyridine and 200 μl of 100 mg/mL PEGDGE 400. The membrane was deposited over the active area (3×1 mm/second dipping) and allowed to cure for two days at 25° C. and 60% Relative Humidity.

TABLE 8
Active Area Composition in 20 mM PBS Buffer
(pH = 7.4)

| | Concentration |
|---|---|
| Component | (mg/mL) |
| GDH Enzyme | 8 |
| Diaphorase | 4 |
| Albumin | 8 |
| NAD⁺ | 8 |
| Glutaraldehyde | 1 |
| Osmium Polymer Complex | 8 |
| PEGDGE 400 | 4 |

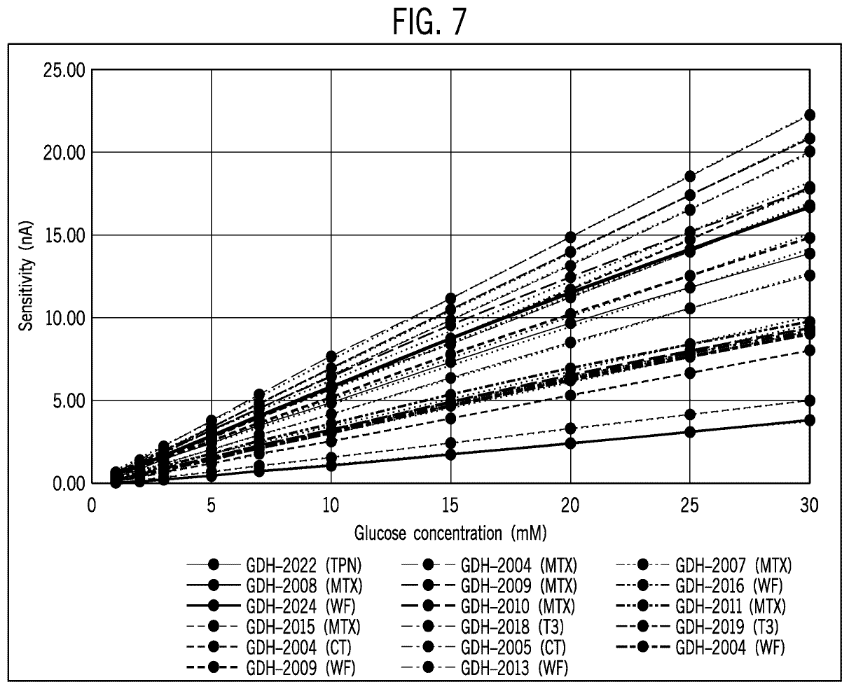

Beaker Calibration. Glucose sensing analyses of the sensor comprising GDH-105 and the sensors comprising protein switches GDH-2004, GDH-2007, GDH-2008, GDH-2009, GDH-2010, GDH-2011, GDH-2015, GDH-2018, GDH-2019, GDH-2005, GDH-2013, GDH-2016, and GDH-2024 as described above was conducted by immersing the electrode in a 100 mM PBS buffer solution at room temperature at varying concentrations of glucose (1, 2, 3, 5, 7, 10, 15, 20, 25, and 30 mM glucose). FIG. 7 shows the response of each of the GDH-105, GDH-2004, GDH-2007, GDH-2008, GDH-2009, GDH-2010, GDH-2011, GDH-2015, GDH-2018, GDH-2019, GDH-2005, GDH-2013, GDH-2016, and GDH-2024 sensors. As shown in Table 9 below, each of the GDHs demonstrate a measurable response to increasing glucose concentrations with sensitivities varying from 0.09 to 0.73 nA/mM glucose. FIG. 7 shows the linear sensitivity response of the GDH-105, GDH-2004, GDH-2007, GDH-2008, GDH-2009, GDH-2010, GDH-2011, GDH-2015, GDH-2018, GDH-2019, GDH-2005, GDH-2013, GDH-2016 and GDH-2024 sensors based on the beaker calibration, demonstrating a positive driving force.

TABLE 9

| | Sensitivity |
|---|---|
| Item ID | (nA/mM Glucose) |
| GDH-105 (control) | 0.55 |
| GDH-2004 (MTX) | 0.30 |
| GDH-2007 (MTX | 0.40 |
| GDH-2008 (MTX) | 0.49 |
| GDH-2009 (MTX) | 0.62 |
| GDH-2010 (MTX) | 0.30 |
| GDH-2011 (MTX) | 0.34 |
| GDH-2015 (MTX) | 0.13 |
| GDH-2018 (T3) | 0.63 |
| GDH-2019 (T3) | 0.67 |
| GDH-2004 (CT) | 0.23 |
| GDH-2005 (CT) | 0.73 |
| GDH-2004 (WF) | 0.28 |
| GDH-2009 (WF) | 0.55 |
| GDH-2013 (WF) | 0.46 |
| GDH-2016 (WF) | 0.56 |
| GDH-2024 (WF) | 0.09 |

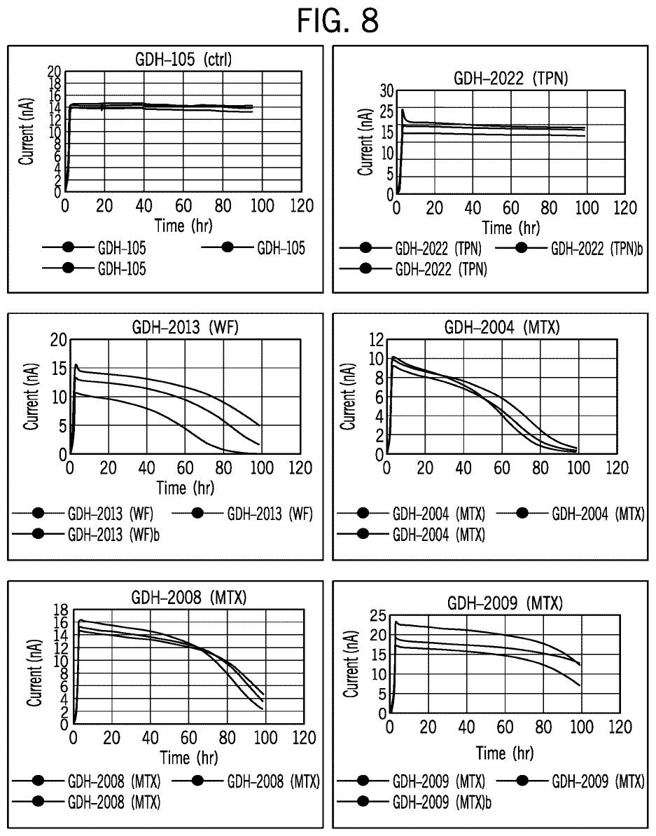

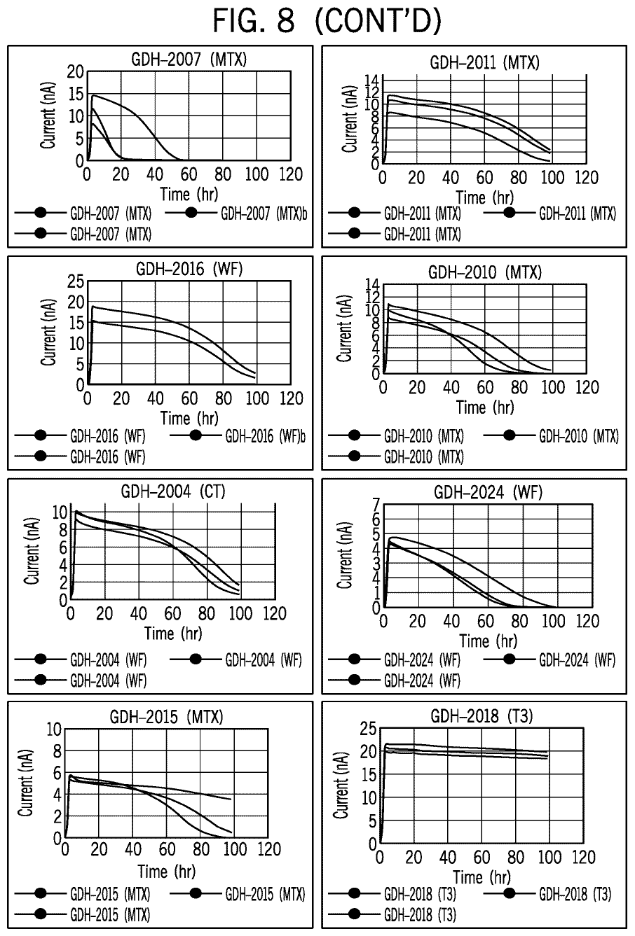

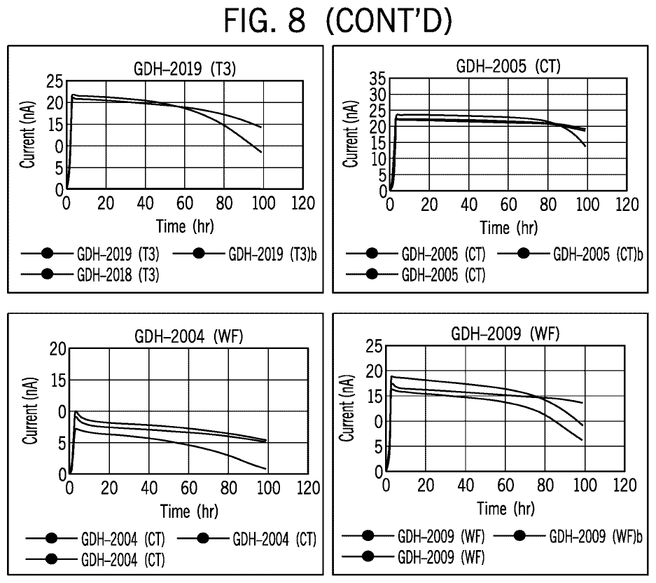

Beaker Stability. The beaker stability (long-term stability) of the sensor GDH-105 and the sensors comprising the protein switches GDH-2004, GDH-2007, GDH-2008, GDH-2009, GDH-2010, GDH-2011, GDH-2015, GDH-2018, GDH-2019, GDH-2005, GDH-2013, GDH-2016, and GDH-2024 described above were evaluated in 30 mM glucose in 100 mM PBS at 33° C. as shown in FIG. 8. After 4 days, each of the signals experienced a sensor drop (decreased stability for detecting glucose) at different rates, as shown in Table 10. The signal drop of the GDH-105 and GDH-2018 sensors was substantially less than that of the other tested sensors.

TABLE 10

| | Signal drop |
|---|---|
| Item ID | (4 days) |
| GDH-105 (control) | −2% |
| GDH-2004 (MTX) | −96% |
| GDH-2007 (MTX | −100% |
| GDH-2008 (MTX) | −77% |
| GDH-2009 (MTX) | −45% |
| GDH-2010 (MTX) | −98% |
| GDH-2011 (MTX) | −85% |
| GDH-2015 (MTX) | −76% |
| GDH-2018 (T3) | −8% |
| GDH-2019 (T3) | −46% |
| GDH-2004 (CT) | −56% |
| GDH-2005 (CT) | −29% |
| GDH-2004 (WF) | −89% |
| GDH-2009 (WF) | −56% |
| GDH-2013 (WF) | −76% |
| GDH-2016 (WF) | −87% |
| GDH-2024 (WF) | −100% |

### Example 4—Warfarin Sensors

Warfarin Sensor. Experimental GDH-105 and GDH-2016 glucose-responsive active areas were prepared using a single-layer active area system. The GDH was obtained from Codexis, Redwood City, CA. The active area was coated onto a carbon working electrode in a single layer composition (Table 8). Following deposition of the active area, the active area was cured for three days at RT.

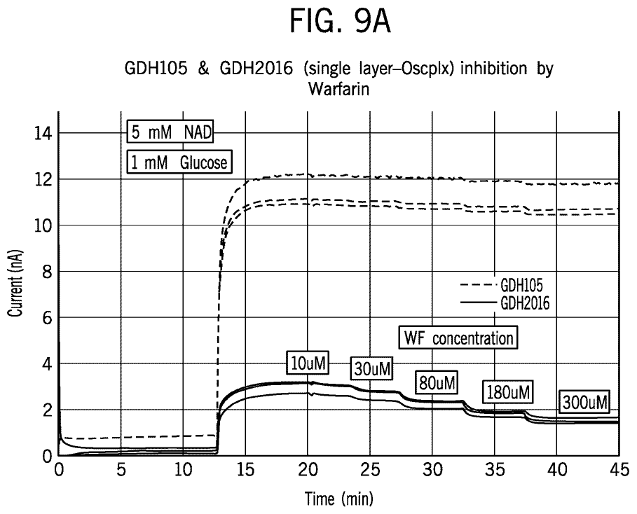

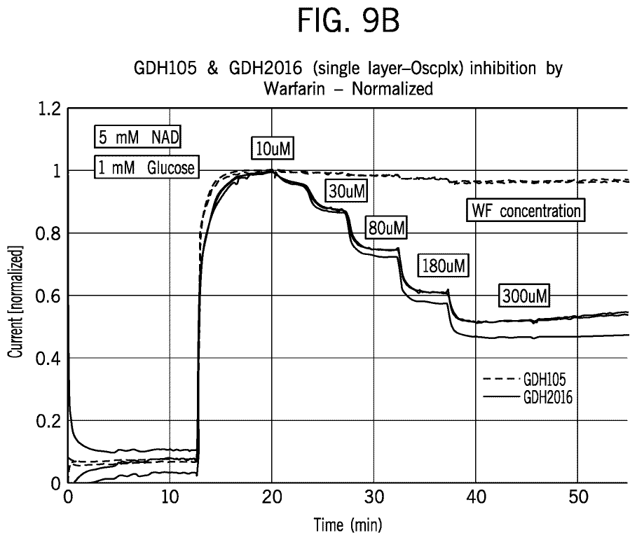

Experimental procedure. Testing of the GDH-105 and GDH-2016 sensors was conducted by immersing the electrode in a 100 mM PBS buffer solution at 33° C. NAD⁺ (5 mM) supplied to a test beaker containing PBS buffer followed by the addition of glucose (1 mM). The GDH-105 and GDH-2016 sensors both showed response to glucose addition. Warfarin was then added to the test beaker at different concentrations (10, 30, 80, 180, and 300 μM warfarin). FIG. 9A and B show the inhibition of the GDH-2016 sensor to increasing warfarin concentrations (˜55% inhibition at 300 μM warfarin), while the GDH-105 sensor was not affected.

Evolved Warfarin Sensors. Experimental GDH-105 (control), GDH-2025 (WF), GDH-2026 (WF), GDH-2027 (WF), and GDH-2028 (WF) glucose-responsive active areas were prepared using a single-layer active area system. The GDH was obtained from Codexis, Redwood City, CA. The active area was coated onto a carbon working electrode in a single layer composition (Table 8). Following deposition of the active area (0.33 mm²), the active area was cured for three days at room temperature. Thereafter, a PVP membrane was applied to the working electrode using a membrane coating solution comprising 4 mL of 100 mg/mL polyvinyl pyridine and 200 μl of 100 mg/mL PEGDGE 400. The membrane was deposited over the active area (3×1 mm/second dipping) and allowed to cure for two days in 25° C. and 60% Relative Humidity.

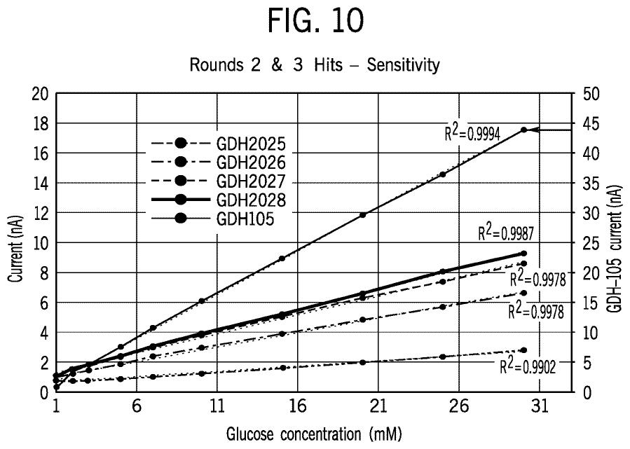

Beaker Calibration. Glucose sensing analyses of the GDH-105, GDH-2025, GDH-2026, GDH-2027, and GDH-2028 sensors prepared as described above was conducted by immersing the electrode in a 100 mM PBS buffer solution at room temperature and varying concentrations of glucose (1, 2, 3, 5, 7, 10, 15, 20, 25, and 30 mM glucose). FIG. 10 shows the response of each of GDH-105, GDH-2025, GDH-2026, GDH-2027, and GDH-2028 sensors. As shown in Table 11, each of the GDHs demonstrated a measurable response to increasing glucose concentrations with sensitivities varying from 0.09 to 1.53 nA/mM glucose. FIG. 10 shows the linear sensitivity response of the GDH-105, GDH-2025, GDH-2026, GDH-2027, and GDH-2028 sensors based on the beaker calibration, demonstrating a positive driving force.

TABLE 11

| | Sensitivity |
|---|---|
| Enzyme ID | (nA/mM) |
| GDH-105 (Ctrl) | 1.53 |
| GDH-2025 (WF) | 0.32 |
| GDH-2026 (WF) | 0.22 |
| GDH-2027 (WF) | 0.29 |
| GDH-2028 (WF) | 0.09 |

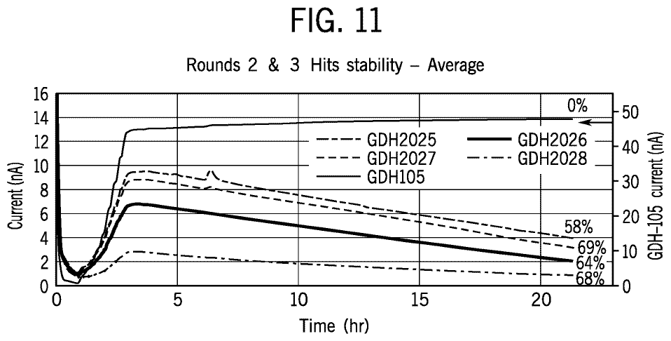

Beaker Stability. The beaker stability (long-term stability) of the GDH-105, GDH-2025, GDH-2026, GDH-2027, and GDH-2028 sensors were evaluated in 30 mM glucose in 100 mM PBS at 33° C. as shown in FIG. 11. After 1 day, each of the signals experience sensor drop (decreased stability for detecting glucose) of different rates, as provided in Table 12. The signal drop of the GDH-105 sensor was substantially less than that of the other tested sensors.

TABLE 12

| | Signal drop |
|---|---|
| Enzyme ID | (1 day) |
| GDH-105 (control) | −0% |
| GDH-2025 (WF) | −58% |
| GDH-2026 (WF) | −69% |
| GDH-2027 (WF) | −64% |
| GDH-2028 (WF) | −68% |

Evolved Warfarin sensors. GDH-105, GDH-2016, GDH-2025, GDH-2026, GDH-2027, and GDH-2028 glucose-responsive active areas were prepared using a single-layer active area system. The GDH was obtained from Codexis, Redwood City, CA. The active area (0.33 mm²) was coated onto a carbon working electrode in a single layer composition (Table 8). Following deposition of the active area, the active area was cured for three days at room temperature.

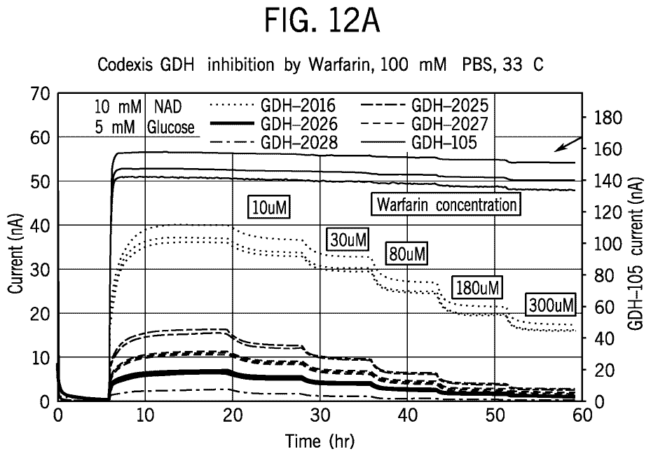

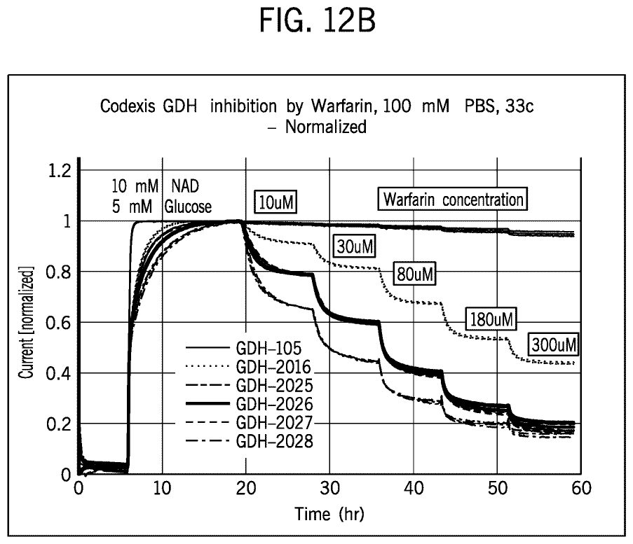

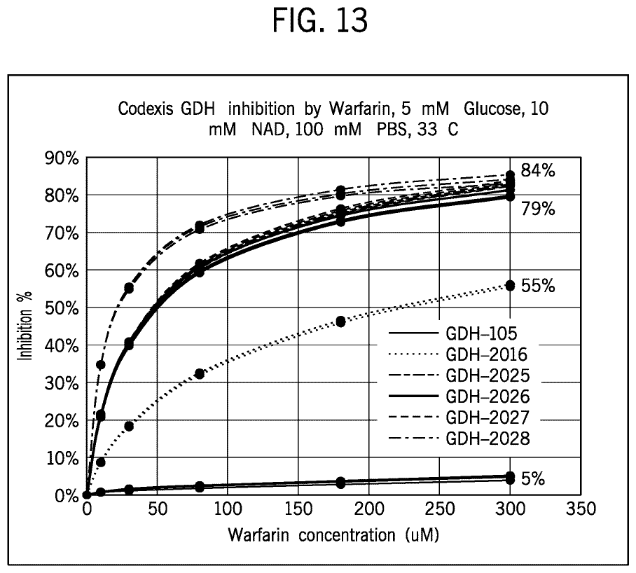

Experimental procedure. Testing of the GDH-105, GDH-2016, GDH-2025, GDH-2026, GDH-2027, and GDH-2028 sensors was conducted by immersing the electrode in a 100 mM PBS buffer solution at 33° C. NAD+ (10 mM) supplied to a test beaker containing PBS buffer followed by the addition of glucose (5 mM). The GDH-105, GDH-2016, GDH-2025, GDH-2026, GDH-2027, and GDH-2028 sensors showed response to glucose addition. Warfarin was then added to the test beaker at different concentrations (10, 30, 80, 180, and 300 μM warfarin). FIGS. 12A and 12B and FIG. 13 show the inhibition of the GDH-105, GDH-2016, GDH-2025, GDH-2026, GDH-2027, and GDH-2028 sensors to increasing warfarin concentrations, while the GDH-105 sensor remained unaffected. Warfarin inhibition percentages varied for the sensors GDH-105, GDH-2025, GDH-2026, GDH-2027, and GDH-2028 as shown in Table 13.

TABLE 13

| | GDH |
|---|---|
| | inhibition |
| | at 0.3 mM |
| Enzyme ID | WF |
| GDH-105 (control) | 5% |
| GDH-2025 (WF) | 79% |
| GDH-2026 (WF) | 78% |
| GDH-2027 (WF) | 79% |
| GDH-2028 (WF) | 84% |

All publications, patents and patent applications discussed and cited herein are incorporated herein by reference in their entireties. It is understood that the aspects disclosed herein are not limited to the particular methodology, protocols and materials described as these can vary. It is also understood that the terminology used herein is for the purposes of describing particular aspects only and is not intended to limit the scope of the present disclosure which will be limited only by the appended claims.

Those skilled in the art will recognize or be able to ascertain using no more than routine experimentation, many equivalents to the specific aspects of the disclosure described herein. Such equivalents are intended to be encompassed by the following claims.

For reasons of completeness, various aspects of the disclosure are set out in the following numbered clauses:
Clause 1. A protein switch comprising: at least one non-naturally occurring polypeptide having (a) at least one analyte binding domain capable of binding with at least one analyte; and (b) at least one oxidase or dehydrogenase domain having oxidase or dehydrogenase activity and capable of binding or reacting with at least one reactant, wherein (i) the analyte that binds to the analyte binding domain is different than the reactant that binds or reacts with the oxidase or dehydrogenase domain; and (ii) when the analyte binds to the analyte binding domain the oxidase or dehydrogenase activity changes.
Clause 2. The protein switch of clause 1, wherein the oxidase is glucose oxidase or lactate oxidase.
Clause 3. The protein switch of clause 1, wherein the dehydrogenase is glucose dehydrogenase or lactate dehydrogenase.
Clause 4. The protein switch of any of clauses 1-3, wherein when the analyte binding domain binds to the analyte, the activity of the oxidase or dehydrogenase is decreased.
Clause 5. The protein switch of any of clauses 1-3, wherein when the analyte binding domain binds to the analyte, the activity of the oxidase or dehydrogenase is increased.
Clause 6. The protein switch of any of clauses 4-5, wherein when the analyte binding domain binds to the analyte, the activity of the oxidase or dehydrogenase is increased or decreased as a result of competitive inhibition, uncompetitive inhibition, or non-competitive inhibition.
Clause 7. The protein switch of any of clauses 4-5, wherein when the analyte binding domain binds to the analyte, the activity of the oxidase or dehydrogenase is decreased as a result of competitive inhibition.
Clause 8. The protein switch of any of clauses 1-7, wherein the analyte is warfarin, cortisol, methotrexate, or triiodothyronine.
Clause 9. The protein switch of any of clauses 1-8, wherein the reactant is glucose or lactate.
Clause 10. A protein switch comprising at least 7 mutations at amino acid positions 96, 155, 156, 159, 170, 198 and 252 of SEQ ID NO:20.
Clause 11. The protein switch of clause 10, wherein the protein switch comprises:
a cysteine, phenylalanine, methionine, tryptophan or tyrosine at amino acid position 96 of SEQ ID NO:20;
an alanine, glycine, isoleucine, leucine or valine at amino acid position 155 of SEQ ID NO:20;
a threonine or serine at amino acid position 156 of SEQ ID NO:20;
a threonine or serine at amino acid position 159 of SEQ ID NO:20;
a lysine, arginine or histidine at amino acid position 170 of SEQ ID NO:20;
a glutamic acid or aspartic acid at amino acid position 198 of SEQ ID NO:20; and
an alanine, glycine, isoleucine, leucine or valine amino acid position 252 of SEQ ID NO:20.
Clause 12. The protein switch of clause 11, further comprising one or more of:
a glycine, isoleucine, leucine or valine at amino acid position 11 of SEQ ID NO:20;
a glycine, isoleucine, leucine or valine at amino acid position 22 of SEQ ID NO:20;
an asparagine or glutamine at amino acid position 45 of SEQ ID NO:20;
an alanine, glycine, isoleucine or leucine at amino acid position 48 of SEQ ID NO:20;
an aspartic acid or glutamic acid at amino acid position 55 of SEQ ID NO:20;
an alanine, glycine, isoleucine, leucine or valine at amino acid position 98 of SEQ ID NO:20;
a phenylalanine, tryptophan, or tyrosine at amino acid position 137 of SEQ ID NO:20;
an alanine, glycine, leucine or valine at amino acid position 141 of SEQ ID NO:20;
an alanine, glycine, isoleucine or leucine at amino acid position 149 of SEQ ID NO:20;
an alanine, glycine, isoleucine or valine at amino acid position 154 of SEQ ID NO:20;
a threonine or serine at amino acid position 166 SEQ ID NO:20;
a glycine, isoleucine, leucine or valine at amino acid position 173 SEQ ID NO:20;
a threonine or serine at amino acid position 184 of SEQ ID NO:20;
a histidine, leucine, or arginine at amino acid position 195 of SEQ ID NO:20;
a threonine or serine at amino acid position 219 of SEQ ID NO:20;
an asparagine or glutamine at amino acid position 240 of SEQ ID NO:20; and/or
an alanine, glycine, isoleucine, leucine or valine at amino acid position 251 at SEQ ID NO:20.
Clause 13. A protein switch comprising an amino acid sequence having at least 80% identity to SEQ ID NO.:11.
Clause 14. The protein switch of clause 13, comprising an amino acid sequence having at least 85% identity to SEQ ID NO.:11.
Clause 15. The protein switch of any of clauses 13-14, comprising an amino acid sequence having at least 90% identity to SEQ ID NO.:11.
Clause 16. The protein switch of any of clauses 13-15, comprising an amino acid sequence having at least 95% identity to SEQ ID NO.:11.
Clause 17. The protein switch of any of clauses 13-16, comprising an amino acid sequence having at least 96% identity to SEQ ID NO.:11.
Clause 18. The protein switch of any of clauses 13-17, comprising an amino acid sequence having at least 97% identity to SEQ ID NO.:11.
Clause 19. The protein switch of any of clauses 13-18, comprising an amino acid sequence having at least 98% identity to SEQ ID NO.:11.
Clause 20. The protein switch of any of clauses 13-19, comprising an amino acid sequence having at least 99% identity to SEQ ID NO.:11.
Clause 21. The protein switch of any of clauses 13-20, comprising an amino acid sequence having at least 100% identity to SEQ ID NO.:11.
Clause 22. The protein switch of any of clauses 13-21, wherein the protein switch comprises an amino acid sequence of any one of SEQ ID NOS. 1-10 or 12-19.
Clause 23. A composition or kit comprising at least one protein switch of clauses 1, 10, 11, or 13 and at least one reactant.
Clause 24. The composition of clause 23, wherein the reactant is glucose or lactate.
Clause 25. A method of detecting an analyte, the method comprising:
providing a protein switch comprising at least one polypeptide having (a) at least one analyte binding domain capable of binding with at least one analyte; and (b) at least one oxidase or dehydrogenase domain having oxidase or dehydrogenase activity and capable of binding or reacting with at least one reactant, wherein (i) the analyte that binds to the analyte binding domain is different than the reactant that binds or reacts with the oxidase or dehydrogenase domain; and (ii) when the analyte binds to the analyte binding domain the oxidase or dehydrogenase activity changes;
contacting the protein switch with a fluid comprising a reactant specific to the protein switch, wherein the analyte binding domain binds to the analyte in the fluid whereby the oxidase or dehydrogenase activity changes; and
detecting a change in a rate of breakdown of the reactant by the oxidase or dehydrogenase domain of the protein switch.
Clause 26. The method of clause 25, wherein the oxidase is glucose oxidase or lactate oxidase.
Clause 27. The method of clause 25, wherein the dehydrogenase is glucose dehydrogenase or lactate dehydrogenase.
Clause 28. The method of any of clauses 26-27, wherein the reactant is glucose or lactate.
Clause 29. A method of detecting an analyte, the method comprising:
providing a protein switch of any of claim 10, 11 or 13,
contacting the protein switch with a fluid comprising at least one reactant specific to the protein switch wherein the analyte binding domain binds to the analyte in the fluid whereby the oxidase or dehydrogenase activity changes; and
detecting a change in a rate of breakdown of the reactant by the oxidase or dehydrogenase domain of the protein switch.
Clause 30. The method of clause 29, wherein the protein switch comprises an amino acid sequence having at least 85% identity to SEQ ID NO.:11.
Clause 31. The method of clauses 29 or 30, wherein the protein switch comprises an amino acid sequence having at least 90% identity to SEQ ID NO.:11.
Clause 32. The method of any of clauses 29-31, wherein the protein switch comprises an amino acid sequence having at least 95% identity to SEQ ID NO.:11.
Clause 33. The method of any of clauses 29-32, wherein the protein switch comprises an amino acid sequence having at least 96% identity to SEQ ID NO.:11.
Clause 34. The method of any of clauses 29-33, wherein the protein switch comprises an amino acid sequence having at least 97% identity to SEQ ID NO.:11.
Clause 35. The method of any of clauses 29-34, wherein the protein switch comprises an amino acid sequence having at least 98% identity to SEQ ID NO.:11.
Clause 36. The method of any of clauses 29-35, wherein the protein switch comprises an amino acid sequence having at least 99% identity to SEQ ID NO.:11.
Clause 37. The method of any of clauses 29-36, wherein the protein switch comprises an amino acid sequence having at least 100% identity to SEQ ID NO.:11.
Clause 38. The method of any of clauses 29-37, wherein the protein switch comprises an amino acid sequence of any one of SEQ ID NOS. 1-10 or 12-19.
Clause 39. The method of any of clauses 29-38, wherein the oxidase is glucose oxidase or lactate oxidase.
Clause 40. The method of any of clauses 29-39, wherein the dehydrogenase is glucose dehydrogenase or lactate dehydrogenase.
Clause 41. The method of any of clauses 29-40, wherein the reactant is glucose or lactate.
Clause 42. The method of any of clauses 29-41, wherein the analyte is warfarin, cortisol, methotrexate, or triiodothyronine.
Clause 43. A system for detecting or monitoring an analyte concentration comprising a sensor control device and a signal detecting device, wherein the sensor control device comprises at least one sensor comprising the protein switch of clauses 1, 10, 11 or 13.
Clause 44. The system of clause 43, wherein the oxidase is glucose oxidase or lactate oxidase.
Clause 45. The system of clause 43, wherein the dehydrogenase is glucose dehydrogenase, or lactate dehydrogenase.
Clause 46. The system of any of clauses 43-45, wherein the protein switch comprises an amino acid sequence having at least 85% identity to SEQ ID NO.:11.
Clause 47. The system of any of clauses 43-46, wherein the protein switch comprises an amino acid sequence having at least 90% identity to SEQ ID NO.:11.
Clause 48. The system of any of clauses 43-47, wherein the protein switch comprises an amino acid sequence having at least 95% identity to SEQ ID NO.:11.
Clause 49. The system of any of clauses 43-48, wherein the protein switch comprises an amino acid sequence having at least 96% identity to SEQ ID NO.:11.
Clause 50. The system of any of clauses 43-49, wherein the protein switch comprises an amino acid sequence having at least 97% identity to SEQ ID NO.:11.
Clause 51. The system of any of clauses 43-50, wherein the protein switch comprises an amino acid sequence having at least 98% identity to SEQ ID NO.:11.
Clause 52. The system of any of clauses 43-51, wherein the protein switch comprises an amino acid sequence having at least 99% identity to SEQ ID NO.:11.
Clause 53. The system of any of clauses 43-52, wherein the protein switch comprises an amino acid sequence having at least 100% identity to SEQ ID NO.:11.
Clause 54. The system any of clauses 43-53, wherein the protein switch comprises an amino acid sequence of any one of SEQ ID NOS. 1-10 or 12-19.
Clause 55. An analyte monitoring system comprising: a sensor comprising a substrate, a working electrode, and the protein switch of clauses 1, 10, 11 or 13, at least a portion of the sensor being adapted for implantation and intimate contact with bodily fluid, the sensor being configured and arranged to produce a signal representative of a level of an analyte in the bodily fluid; and a signal detecting device for receiving the signal, wherein said signal is generated by contact of said analyte with said protein switch.
Clause 56. The analyte monitoring system of clause 55, wherein the oxidase is glucose oxidase or lactate oxidase.
Clause 57. The analyte monitoring system of clause 55, wherein the dehydrogenase is glucose dehydrogenase or lactate dehydrogenase.
Clause 58. The analyte monitoring system of clauses 55-57, wherein the protein switch comprises an amino acid sequence having at least 85% identity to SEQ ID NO.:11.
Clause 59. The analyte monitoring system of clauses 55-58, wherein the protein switch comprises an amino acid sequence having at least 90% identity to SEQ ID NO.:11.
Clause 60. The analyte monitoring system of clauses 55-59, wherein the protein switch comprises an amino acid sequence having at least 95% identity to SEQ ID NO.:11.
Clause 61. The analyte monitoring system of clauses 55-60, wherein the protein switch comprises an amino acid sequence having at least 96% identity to SEQ ID NO.:11.
Clause 62. The analyte monitoring system of clauses 55-61, wherein the protein switch comprises an amino acid sequence having at least 97% identity to SEQ ID NO.:11.
Clause 63. The analyte monitoring system of clauses 55-62, wherein the protein switch comprises an amino acid sequence having at least 98% identity to SEQ ID NO.:11.
Clause 64. The analyte monitoring system of clauses 55-63, wherein the protein switch comprises an amino acid sequence having at least 99% identity to SEQ ID NO.:11.
Clause 65. The analyte monitoring system of clauses 55-64, wherein the protein switch comprises an amino acid sequence having at least 100% identity to SEQ ID NO.:11.
Clause 66. The analyte monitoring system of clauses 55-65, wherein the protein switch comprises an amino acid sequence of any one of SEQ ID NOS. 1-10 or 12-19.
Clause 67. The analyte monitoring system of clauses 55-66, wherein the analyte is warfarin, cortisol, methotrexate, or triiodothyronine.

What is claimed is:

1. A protein switch comprising: at least one non-naturally occurring polypeptide having (a) at least one analyte binding domain capable of binding with at least one analyte; and (b) at least one oxidase or dehydrogenase domain having oxidase or dehydrogenase activity and capable of binding or reacting with at least one reactant, wherein (i) the analyte that binds to the analyte binding domain is different than the reactant that binds or reacts with the oxidase or dehydrogenase domain; and (ii) when the analyte binds to the analyte binding domain the oxidase or dehydrogenase activity changes.

2. The protein switch of claim 1, wherein the oxidase is glucose oxidase or lactate oxidase.

3. The protein switch of claim 1, wherein the dehydrogenase is glucose dehydrogenase or lactate dehydrogenase.

4. The protein switch of any of claims 1-3, wherein when the analyte binding domain binds to the analyte, the activity of the oxidase or dehydrogenase is decreased.

5. The protein switch of any of claims 1-3, wherein when the analyte binding domain binds to the analyte, the activity of the oxidase or dehydrogenase is increased.

6. The protein switch of any of claims 4-5, wherein when the analyte binding domain binds to the analyte, the activity of the oxidase or dehydrogenase is increased or decreased as a result of competitive inhibition, uncompetitive inhibition, or non-competitive inhibition.

7. The protein switch of any of claims 4-5, wherein when the analyte binding domain binds to the analyte, the activity of the oxidase or dehydrogenase is decreased as a result of competitive inhibition.

8. The protein switch of any of claims 1-7, wherein the analyte is warfarin, cortisol, methotrexate, or triiodothyronine.

9. The protein switch of any of claims 1-8, wherein the reactant is glucose or lactate.

10. A protein switch comprising at least 7 mutations at amino acid positions 96, 155, 156, 159, 170, 198 and 252 of SEQ ID NO:20.

11. The protein switch of claim 10, wherein the protein switch comprises:
a cysteine, phenylalanine, methionine, tryptophan or tyrosine at amino acid position 96 of SEQ ID NO:20;
an alanine, glycine, isoleucine, leucine or valine at amino acid position 155 of SEQ ID NO:20;
a threonine or serine at amino acid position 156 of SEQ ID NO:20;
a threonine or serine at amino acid position 159 of SEQ ID NO:20;
a lysine, arginine or histidine at amino acid position 170 of SEQ ID NO:20;
a glutamic acid or aspartic acid at amino acid position 198 of SEQ ID NO:20; and
an alanine, glycine, isoleucine, leucine or valine amino acid position 252 of SEQ ID NO:20.

12. The protein switch of claim 11, further comprising one or more of:
a glycine, isoleucine, leucine or valine at amino acid position 11 of SEQ ID NO:20;
a glycine, isoleucine, leucine or valine at amino acid position 22 of SEQ ID NO:20;
an asparagine or glutamine at amino acid position 45 of SEQ ID NO:20;
an alanine, glycine, isoleucine or leucine at amino acid position 48 of SEQ ID NO:20;
an aspartic acid or glutamic acid at amino acid position 55 of SEQ ID NO:20;
an alanine, glycine, isoleucine, leucine or valine at amino acid position 98 of SEQ ID NO:20;
a phenylalanine, tryptophan, or tyrosine at amino acid position 137 of SEQ ID NO:20;
an alanine, glycine, leucine or valine at amino acid position 141 of SEQ ID NO:20;
an alanine, glycine, isoleucine or leucine at amino acid position 149 of SEQ ID NO:20;
an alanine, glycine, isoleucine or valine at amino acid position 154 of SEQ ID NO:20;
a threonine or serine at amino acid position 166 SEQ ID NO:20;
a glycine, isoleucine, leucine or valine at amino acid position 173 SEQ ID NO:20;
a threonine or serine at amino acid position 184 of SEQ ID NO:20;
a histidine, leucine, or arginine at amino acid position 195 of SEQ ID NO:20;
a threonine or serine at amino acid position 219 of SEQ ID NO:20;
an asparagine or glutamine at amino acid position 240 of SEQ ID NO:20; and/or
an alanine, glycine, isoleucine, leucine or valine at amino acid position 251 at SEQ ID NO:20.

13. A protein switch comprising an amino acid sequence having at least 80% identity to SEQ ID NO.:11.

14. The protein switch of claim 13, comprising an amino acid sequence having at least 85% identity to SEQ ID NO.:11.

15. The protein switch of any of claims 13-14, comprising an amino acid sequence having at least 90% identity to SEQ ID NO.:11.

16. The protein switch of any of claims 13-15, comprising an amino acid sequence having at least 95% identity to SEQ ID NO.:11.

17. The protein switch of any of claims 13-16, comprising an amino acid sequence having at least 96% identity to SEQ ID NO.:11.

18. The protein switch of any of claims 13-17, comprising an amino acid sequence having at least 97% identity to SEQ ID NO.:11.

19. The protein switch of any of claims 13-18, comprising an amino acid sequence having at least 98% identity to SEQ ID NO.:11.

20. The protein switch of any of claims 13-19, comprising an amino acid sequence having at least 99% identity to SEQ ID NO.:11.

21. The protein switch of any of claims 13-20, comprising an amino acid sequence having at least 100% identity to SEQ ID NO.:11.

22. The protein switch of any of claims 13-21, wherein the protein switch comprises an amino acid sequence of any one of SEQ ID NOS. 1-10 or 12-19.

23. A composition or kit comprising at least one protein switch of claim 1, 10, 11 or 13 and at least one reactant.

24. The composition of claim 23, wherein the reactant is glucose or lactate.

25. A method of detecting an analyte, the method comprising:
providing a protein switch comprising at least one polypeptide having (a) at least one analyte binding domain capable of binding with at least one analyte; and (b) at least one oxidase or dehydrogenase domain having oxidase or dehydrogenase activity and capable of binding or reacting with at least one reactant, wherein (i) the analyte that binds to the analyte binding domain is different than the reactant that binds or reacts with the oxidase or dehydrogenase domain; and (ii) when the analyte binds to the analyte binding domain the oxidase or dehydrogenase activity changes;
contacting the protein switch with a fluid comprising a reactant specific to the protein switch wherein the analyte binding domain binds to the analyte in the fluid whereby the oxidase or dehydrogenase activity changes; and
detecting a change in a rate of breakdown of the reactant by the oxidase or dehydrogenase domain of the protein switch.

26. The method of claim 25, wherein the oxidase is glucose oxidase or lactate oxidase.

27. The method of claim 25, wherein the dehydrogenase is glucose dehydrogenase or lactate dehydrogenase.

28. The method of any of claims 26-27, wherein the reactant is glucose or lactate.

29. A method of detecting an analyte, the method comprising:
providing a protein switch of any of claim 10, 11 or 13;
contacting the protein switch with a fluid comprising a reactant specific to the protein switch, wherein the analyte binding domain binds to the analyte in the fluid whereby the oxidase or dehydrogenase activity changes; and
detecting a change in a rate of breakdown of the reactant by the oxidase or dehydrogenase domain of the protein switch.

30. The method of claim 29, wherein the protein switch comprises an amino acid sequence having at least 85% identity to SEQ ID NO.:11.

31. The method of claim 29 or 30, wherein the protein switch comprises an amino acid sequence having at least 90% identity to SEQ ID NO.:11.

32. The method of any of claims 29-31, wherein the protein switch comprises an amino acid sequence having at least 95% identity to SEQ ID NO.:11.

33. The method of any of claims 29-32, wherein the protein switch comprises an amino acid sequence having at least 96% identity to SEQ ID NO.:11.

34. The method of any of claims 29-33, wherein the protein switch comprises an amino acid sequence having at least 97% identity to SEQ ID NO.:11.

35. The method of any of claims 29-34, wherein the protein switch comprises an amino acid sequence having at least 98% identity to SEQ ID NO.:11.

36. The method of any of claims 29-35, wherein the protein switch comprises an amino acid sequence having at least 99% identity to SEQ ID NO.:11.

37. The method of any of claims 29-36, wherein the protein switch comprises an amino acid sequence having at least 100% identity to SEQ ID NO.:11.

38. The method of any of claims 29-37, wherein the protein switch comprises an amino acid sequence of any one of SEQ ID NOS. 1-10 or 12-19.

39. The method of any of claims 29-38, wherein the oxidase is glucose oxidase or lactate oxidase.

40. The method of any of claims 29-39, wherein the dehydrogenase is glucose dehydrogenase or lactate dehydrogenase.

41. The method of any of claims 29-40, wherein the reactant is glucose or lactate.

42. The method of any of claims 29-41, wherein the analyte is warfarin, cortisol, methotrexate, or triiodothyronine.

43. A system for detecting or monitoring an analyte concentration comprising a sensor control device and a signal detecting device, wherein the sensor control device comprises at least one sensor comprising the protein switch of claim 1, 10, 11 or 13.

44. The system of claim 43, wherein the oxidase is glucose oxidase or lactate oxidase.

45. The system of claim 43, wherein the dehydrogenase is glucose dehydrogenase, or lactate dehydrogenase.

46. The system of any of claims 43-45, wherein the protein switch comprises an amino acid sequence having at least 85% identity to SEQ ID NO.:11.

47. The system of any of claims 43-46, wherein the protein switch comprises an amino acid sequence having at least 90% identity to SEQ ID NO.:11.

48. The system of any of claims 43-47, wherein the protein switch comprises an amino acid sequence having at least 95% identity to SEQ ID NO.:11.

49. The system of any of claims 43-48, wherein the protein switch comprises an amino acid sequence having at least 96% identity to SEQ ID NO.:11.

50. The system of any of claims 43-49, wherein the protein switch comprises an amino acid sequence having at least 97% identity to SEQ ID NO.:11.

51. The system of any of claims 43-50, wherein the protein switch comprises an amino acid sequence having at least 98% identity to SEQ ID NO.:11.

52. The system of any of claims 43-51, wherein the protein switch comprises an amino acid sequence having at least 99% identity to SEQ ID NO.:11.

53. The system of any of claims 43-52, wherein the protein switch comprises an amino acid sequence having at least 100% identity to SEQ ID NO.:11.

54. The system any of claims 43-53, wherein the protein switch comprises an amino acid sequence of any one of SEQ ID NOS. 1-10 or 12-19.

55. An analyte monitoring system comprising: a sensor comprising a substrate, a working electrode, and the protein switch of claim 1, 10, 11 or 13, at least a portion of the sensor being adapted for implantation and intimate contact with bodily fluid, the sensor being configured and arranged to produce a signal representative of a level of an analyte in the bodily fluid; and a signal detecting device for receiving the signal, wherein said signal is generated by contact of said analyte with said protein switch.

56. The analyte monitoring system of claim 55, wherein the oxidase is glucose oxidase or lactate oxidase.

57. The analyte monitoring system of claim 55, wherein the dehydrogenase is glucose dehydrogenase or lactate dehydrogenase.

58. The analyte monitoring system of claims 55-57, wherein the protein switch comprises an amino acid sequence having at least 85% identity to SEQ ID NO.:11.

59. The analyte monitoring system of claims 55-58, wherein the protein switch comprises an amino acid sequence having at least 90% identity to SEQ ID NO.:11.

60. The analyte monitoring system of claims 55-59, wherein the protein switch comprises an amino acid sequence having at least 95% identity to SEQ ID NO.:11.

61. The analyte monitoring system of claims 55-60, wherein the protein switch comprises an amino acid sequence having at least 96% identity to SEQ ID NO.:11.

62. The analyte monitoring system of claims 55-61, wherein the protein switch comprises an amino acid sequence having at least 97% identity to SEQ ID NO.:11.

63. The analyte monitoring system of claims 55-62, wherein the protein switch comprises an amino acid sequence having at least 98% identity to SEQ ID NO.:11.

64. The analyte monitoring system of claims 55-63, wherein the protein switch comprises an amino acid sequence having at least 99% identity to SEQ ID NO.:11.

65. The analyte monitoring system of claims 55-64, wherein the protein switch comprises an amino acid sequence having at least 100% identity to SEQ ID NO.:11.

66. The analyte monitoring system of claims 55-65, wherein the protein switch comprises an amino acid sequence of any one of SEQ ID NOS. 1-10 or 12-19.

67. The analyte monitoring system of claims 55-66, wherein the analyte is warfarin, cortisol, methotrexate, or triiodothyronine.
